(19) United States
(12) Patent Application Publication
(10) Pub. No.: US 2011/0009478 A1
(43) Pub. Date: Jan. 13, 2011

(54) THERANOSTIC POLYCATION BEACONS COMPRISING OLIGOETHYLENEAMINE REPEATING UNITS AND LANTHANIDE CHELATES

(76) Inventors: Theresa M. REINEKE, Blacksburg, VA (US); Joshua M. Bryson, Blacksburg, VA (US)

Correspondence Address: NEW RIVER VALLEY INTELLECTUAL PROPERTY LAW, P.O. BOX 10944, BLACKSBURG, VA, 24062, US

(21) Appl. No.: 12/832,915

(22) Filed: Jul. 8, 2010

Related U.S. Application Data
(60) Provisional application No. 61/223,812, filed on Jul. 8, 2009.

(51) Int. Cl. A61K 48/00; C12N 5/02; C12Q 1/68; C07C 229/16; A61P 31/00; A61P 37/04; C08G 69/10

(52) U.S. Cl. 514/44 R; 435/375; 435/6; 562/565; 528/328

(57) ABSTRACT

The present invention relates to vehicles for delivering macromolecules into cells. More particularly, embodiments of the invention relate to compounds and methods for binding and compacting nucleic acids into nanoparticles for transferring the polynucleotides into cells and which can be configured to provide a mechanism for visualization of the delivery vehicles on the nm/μm scale by microscopy and on the sub-mm scale by magnetic resonance imaging. Polycations according to embodiments of the invention have been designed to contain repeated oligoethyleneamines, for binding and compacting nucleic acids into nanoparticles, and lanthanide (Ln) chelates (for example, using luminescent europium Eu³⁺ and paramagnetic gadolinium Gd³⁺). Preferred polymeric imaging beacons according to embodiments of the invention comprise repeating units of metal chelates within an oligoamine backbone, the repeating units comprising:

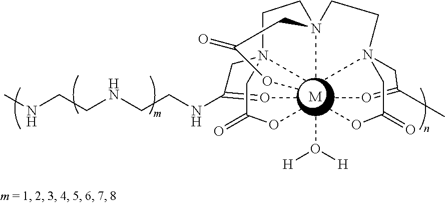

wherein n ranges from 2 to 10,000,000;
M is a metal capable of exhibiting an imaging functionality for an imaging modality; and
the oligoamine backbone comprises from 1 to 8 ethlyeneamines and is chosen from diethyleneamine, triethylenediamine, tetraethylenetriamine, pentaethylenetetraamine, hexaethylenepentamine, heptaethylenehexamine, octaethyleneheptamine or nonethyleneoctaamine. Especially preferred, are structures chelated with either Eu3+ or Gd3+ for microscopy or MRI imaging, respectively. Such chelated lanthanides allow for the visualization of the delivery vehicle both on the nm/μm scale by way of microscopy and on the sub-mm scale using MRI.

### CROSS-REFERENCE TO RELATED APPLICATIONS

This application relies on the disclosure of and claims the benefit of the filing date of U.S. Provisional Application No. 61/223,812, filed Jul. 8, 2009, the disclosure of which is incorporated by reference herein in its entirety.

### BACKGROUND OF THE INVENTION

1. Field of the Invention

The present invention relates to vehicles for delivering macromolecules into cells. More particularly, embodiments of the invention relate to compounds and methods for binding and compacting nucleic acids into nanoparticles for transferring the polynucleotides into cells and which can be configured to provide a mechanism for visualization of the delivery vehicles on the nm/μm scale by microscopy and on the sub-mm scale by magnetic resonance imaging. This technology can also be used for delivering drugs, proteins, peptides and other related technologies.

2. Description of the Related Art

Although materials have been developed and studied for polynucleotide transfer, the biological mechanisms and fate of the synthetic vehicle has remained elusive due to the limitations with current labeling technologies.

The delivery of therapeutic nucleic acids such as siRNA, antisense agents, transcription factor decoys, and plasmid (p)DNA offers an unprecedented opportunity for developing highly specific treatments for many devastating diseases. See Leong P L, et al. (2003) Targeted inhibition of Stat3 with a decoy oligonucleotide abrogates head and neck cancer cell growth, Proc Natl Acad Sci USA 100:4138-4143; and Heidel J D, et al. (2007) Administration in non-human primates of escalating intravenous doses of targeted nanoparticles containing ribonucleotide reductase subunit M2 siRNA, Proc Natl Acad Sci USA 104:5715-5721.

The use of synthetic materials, such as polymers, for polynucleotide delivery has rapidly grown, and presents a wealth of promising alternatives to conventional viral vectors, which have caused serious problems in the clinic. See, e.g., Schaffert D, Wagner E (2008) Gene therapy progress and prospects: Synthetic polymer-based systems, Gene Ther 15:1131-1138; Liu Y, Reineke T M (2005) Hydroxyl stereochemistry and amine number within poly(glycol-amidoamine)s affect intracellular DNA delivery, J Am Chem Soc 127:3004-3015; Srinivasachari S, Liu Y, Zhang G, Prevette L, Reineke T M (2006) Trehalose click polymers inhibit nanoparticle aggregation and promote pDNA delivery in serum, J Am Chem Soc 128:8176-84, the disclosures of which are incorporated by reference herein in their entireties.

Lanthanide (Ln) metals are endowed with several unique properties that can be exploited for the development and study of nonviral delivery vehicles. Complexes housing luminescent lanthanides, such as europium Eu³⁺, offer many unique advantages over the aforementioned labeling methods due to their small hydrophilic structures, long luminescence lifetimes, large Stokes shifts, and their stability from quenching and photobleaching. See Bünzli J-CG (2006) Benefiting from the unique properties of lanthanide ions, Acc Chem Res 39:53-61; and Marriott G, Heidecker M, Diamandis E P, Yan-Marriott Y (1994) Time-resolved delayed luminescence image microscopy using an europium ion chelate complex, Biophys J 67:957-965.

The chelates of lanthanides are relatively nontoxic; structures containing gadolinium Gd³⁺ are FDA-approved as MRI contrast agents due to their paramagnetic nature and slow electronic relaxation time. See Caravan P, Ellison J J, McMurray T J, Lauffer R B (1999) Gadolinium(III) chelates as MRI contrast agents: Structure, dynamics, and applications, Chem Rev 99:2293-2352. Polymers and macromolecules containing gadolinium chelates are being studied as new contrast agents, because their large structures increase the rotational correlation time, which improves resolution and sensitivity. See Bryant H L, et al. (1999) Synthesis and relaxometry of high-generation (G=5, 7, 9, and 10) PAMAM dendrimer-DOTA-gadolinium chelates, J Magn Reson Imaging 9:348-352. Also, MRI is advantageous for following drug delivery in vivo with very high resolution, because it is a noninvasive and safe imaging method. See Chen H H, et al. (2005) MR imaging of biodegradable polymeric microparticles: A potential method of monitoring local drug delivery, Magn Reson Med 53:614-620.

Although much is known about the infection pathways, advantages, and troubles of viral vectors, researchers in the field of nonviral delivery are just beginning to understand the transfection mechanisms, benefits, and potential issues with the multitude of materials being developed as macromolecular drug carriers. See, e.g., Thomas C E, Ehrhardt A, Kay M A (2003) Progress and problems with the use of viral vectors for gene therapy, Nat Rev Genet. 4:346-358; Karmali P P, Chaudhuri A (2007) Cationic liposomes as non-viral carriers of gene medicines: Resolved issues, open questions, and future promises, Med Res Rev 27:696-722; and Sonawane N D, Szoka F C, Verkman A S (2003) Chloride accumulation and swelling in endosomes enhances DNA transfer by polyamine-DNA polyplexes, J Biol Chem 278:44826-44831.

Considering that the delivery vehicle has a central role in the mechanisms, kinetics, efficacy, and toxicity of nucleic acid medicines, little is known about how the vehicle structure affects drug fate both in vitro and in vivo. For this reason, smart biomaterials termed “theranostic” agents are being developed that provide diagnostic imaging, therapeutic delivery, and the ability to monitor treatment efficacy. See Pan D, et al. (2008) Ligand-directed nanobialys as theranostic agents for drug delivery and manganese-based magnetic resonance imaging of vascular targets, J Am Chem Soc 130:9186-9187.

Indeed, the parallel development of novel nucleic acid drugs and theranostic vehicles that offer disease diagnosis, treatment, and the ability to understand the delivery mechanisms/kinetics on a range of biological scales will advance this field toward the discovery of personalized treatment strategies. Tracking the delivery of nucleic acids within cells and tissues has traditionally been accomplished by a number of methods such as labeling nucleotides with fluorescent dyes, radiotracers, quantum dots, and/or with various reporter gene assays. See, e.g., Sonawane (2003) above; Malik N, et al. (2000) Dendrimers: Relationship between the structure and biocompatibility in vitro, and preliminary studies on the biodistribution of 1251-labelled polyamidoamine dendrimers in vivo, J Control Release 65:133-148; Resch-Genger U, Grabolle M, Cavaliere-Jaricot S, Nitschke R, Nann T (2008) Quantum dots versus organic dyes as fluorescent labels. Nat Methods 5:763-775; Qi L, Gao X (2008) Quantum dot-amphipol nanocomplex for intracellular delivery and real-time imaging of siRNA. ACS Nano 2:1403-1410; and Liu & Reineke (2005) above.

Although these methods have yielded a means of monitoring the presence and location of nucleic acids, many issues have surfaced with labeling polymeric delivery vehicles with these techniques. For example, the polymer labeling efficiency is often poor, non-uniform, irreproducible, and difficult to characterize. Also, dyes can alter the delivery mechanisms and/or increase side-effects. See Resch-Genger (2008) above.

For this reason, the development of new material-based delivery systems that allow monitoring of both the nucleic acid and the delivery vehicle, on the cellular and tissue scales, is essential to improve the delivery efficiency, optimize the vehicle structure, and monitor treatment efficacy in living systems.

### SUMMARY OF THE INVENTION

The delivery of nucleic acids with polycations offers tremendous potential for developing highly specific treatments for various therapeutic targets. To this end, the inventors have developed polymer beacons that allow the delivery of nucleic acids to be visualized at different biological scales.

The polycations have been designed to contain repeated oligoethyleneamines, for binding and compacting nucleic acids into nanoparticles, and lanthanide (Ln) chelates (for example, using luminescent europium Eu³⁺ and paramagnetic gadolinium Gd³⁺). The chelated lanthanides allow the visualization of the delivery vehicle both on the nm/μm scale by way of microscopy and on the sub-mm scale using MRI. These compounds have been described by the inventors in Polymer Beacons for Luminescence and Magnetic Resonance Imaging of DNA Delivery, PNAS, vol. 106, no. 40 (2009), the disclosure of which is incorporated by reference herein in its entirety, including the referenced supporting information online at www.pnas.org/cgi/content/full/0904860106/DCSupplemental.

FIG. 1A is a schematic of two analogous polymeric imaging beacons differing in ethyleneamine length (3a or 3b contain 3 or 4 ethyleneamines, respectively). The structures can be chelated with either Eu3+ or Gd3+ for microscopy or MRI imaging, respectively.

These delivery beacons effectively bind and compact plasmid (p)DNA into nanoparticles and protect nucleic acids from nuclease damage. Further, these delivery beacons efficiently deliver pDNA into cultured cells and do not exhibit toxicity. Micrographs of cultured cells exposed to the nanoparticle complexes formed with fluorescein-labeled pDNA and the europium-chelated polymers reveal effective intracellular imaging of the delivery process. MRI of bulk cells exposed to the complexes formulated with pDNA and the gadolinium-chelated structures show bright image contrast, allowing visualization of effective intracellular delivery on the tissuescale. Because of their versatility, these delivery beacons posses remarkable potential for tracking and understanding nucleic acid transfer in vitro, and have promise as in vivo theranostic agents.

### BRIEF DESCRIPTION OF THE DRAWINGS

FIG. 1A provides a schematic diagram of preferred embodiments of polymeric imaging beacons according to the present invention.

FIG. 1B provides a schematic diagram of general exemplary polymeric imaging beacons according to embodiments of the present invention.

FIG. 1C provides a schematic diagram of unchelated polymers according to embodiments of the present invention.

FIGS. 2A-F are photographs of agarose gel electrophoresis shift assays showing Gd3a (FIG. 2A), Gd3b (FIG. 2B), Eu3a (FIG. 2D), and Eu3b (FIG. 2E) binding with pDNA at increasing N/P ratios from 0 to 40.

FIGS. 3A-B are TEM micrographs of polyplex Eu3a and Eu3b, respectively.

FIG. 3C is a graph showing DLS (bars) and zeta potentials (lines) of polyplexes at various N/P ratios (the average and SD of three measurements are shown).

FIG. 4 provides a series of photographs of gel electrophoresis assays showing the stability of the polyplexes from nuclease degradation, i.e., pDNA integrity.

FIG. 5 is a graph showing the results of Luciferase Expression in HeLa cells.

FIGS. 6A-B are graphs showing the effect of lanthanide chelate and N/P ratio on polyplex uptake into Hela cells and cell viability.

FIG. 7 is a graph showing cell viability after exposure to polyplexes using unlabeled pDNA when studied by MTT assay.

FIGS. 8A-D are deconvoluted micrographs of a HeLa cell transfected with FITCpDNA/Eu3a polyplexes.

FIGS. 9A-D are deconvoluted micrographs of a HeLa cell transfected with FITCpDNA/Eu3b polyplexes.

FIGS. 10A-D are two-photon confocal images of FITC-labeled pDNA delivered with Eu3a at an N/P of 20.

FIGS. 11A-B are graphs showing the relaxivities and relaxation rate constants of water solutions containing dissolved polymer and polyplexes, respectively.

FIGS. 12A-B are images showing the MRI data for cells transfected with Gd3a/pDNA and Gd3b/pDNA polyplexes.

### DETAILED DESCRIPTION OF VARIOUS EMBODIMENTS OF THE INVENTION

Reference will now be made in detail to various exemplary embodiments of the invention. The following detailed description is presented for the purpose of describing certain embodiments in detail and is, thus, not to be considered as limiting the invention to the embodiments described.

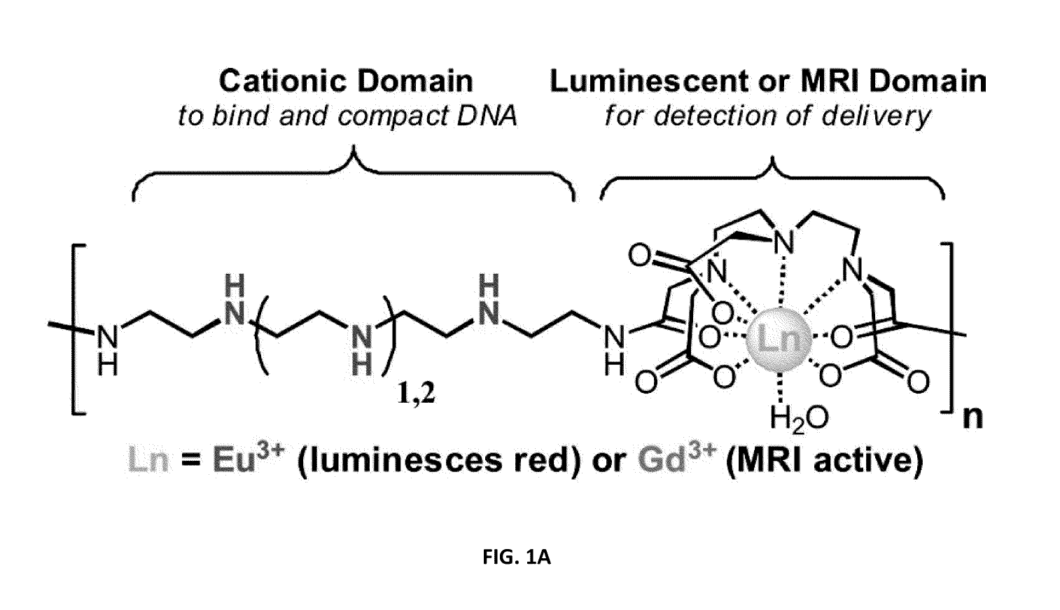

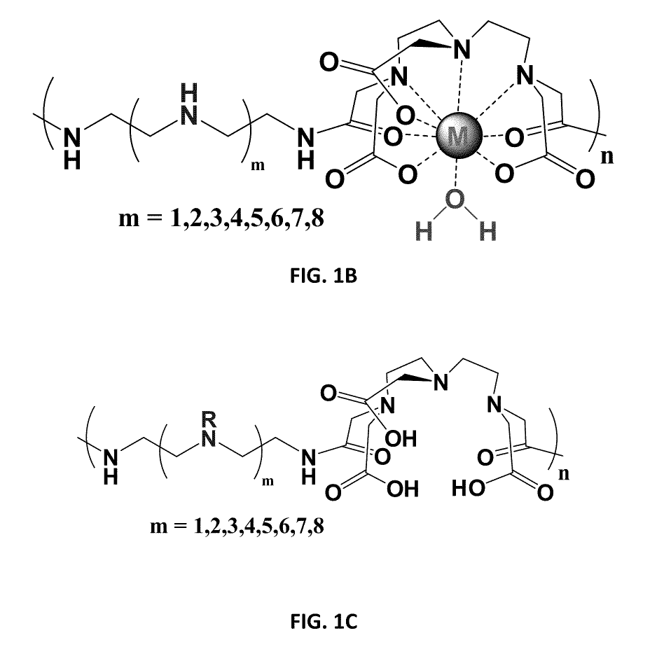

The polymer delivery vehicles developed here offer a creative and powerful method for tracking the delivery of nucleic acids. For example, FIG. 1A provides a schematic illustration of representative delivery beacons according to embodiments of the invention, which have been designed to contain systematically repeated lanthanide chelates within an oligoethyleneamine backbone. The inventors have found that these materials can bind and compact pDNA into nanoparticles (termed polyplexes) that are taken up into cultured human cervix adenocarcinoma (HeLa) cells, for example, in an effective and nontoxic manner.

Polycations self-assemble with biologically active molecules, and in particular nucleic acids and peptides, through electrostatic interactions and they compact DNA into small complexes that have been termed polyplexes. This has previously been disclosed in U.S. Pat. No. 5,948,878, Burgess et al., which is herein incorporated by reference in its entirety. The formation of the polyplexes usually occurs at a N/P ratio [the ratio of polymer nitrogens (N) to phosphate groups (P) and the DNA] greater than one. Polyplexes can be taken be taken up by the cell by through the endocytic pathway. After endocytosis the polyplexes are able to escape the endosomes and are able to enter the nucleus where the delivered gene is transcribed and translated into the desired protein. The polymers can be used to deliver any type, length, sequence, and shape of molecule (e.g., nucleic acid) to any cellular destination.

The polymer structure plays a large role in the binding affinity of DNA and the compaction of DNA into polyplexes. Also, the polymer chemistry dictates the efficiency of polyplex cellular uptake and endosomal release within the cytoplasm. Furthermore, the polymer structure has been shown to significantly affect both the delivery efficiency and toxicity that is observed during transport of the biologically active portion of the complex.

The oligoamine repeating units can be diethyleneamine, triethylenediamine, tetraethylenetriamine (shown as the first option for the cationic domain of FIG. 1A), pentaethylenetetraamine (shown as the second option for the cationic domain of FIG. 1A), hexaethylenepentamine, heptaethylenehexamine, octaethyleneheptamine, or nonethyleneoctaamine. More specifically, FIG. 1A provides examples (3a, 3b, respectively) in which there are 3 ethyleneamines (or a tetraethylenetriamine portion) and in which there are 4 ethyleneamines (or a pentaethylenetetraamine portion).

As used herein, the term “repeat unit” or “repeating unit” refers to the identical combining units that are joined to form a polymer. Thus, the number of repeat units of a polymer is indicative of its degree of polymerization.

The degree of polymerization (see “n” in FIG. 1A) for the polyplexes of embodiments of the invention can be 2 or higher and can range up to about 500,000; such as up to about 1,000,000; or up to about 250,000; up to about 100,000; up to about 50,000; up to about 25,000; up to about 10,000; up to about 5,000; up to about 1,000; up to about 500; or up to about 100 to name a few examples. Indeed any amount of polymerization can be achieved depending on the desired result and by modifying the polymerization conditions.

One of ordinary skill in the art will appreciate that the reaction parameters (such as catalyst concentration, polymerization time, and temperature) may be varied to provide polymers having different degrees of polymerization.

As for the luminescent or MRI domain portion of the compound, any lanthanide metal can be used as a chelate, including lanthanum, cerium, praseodymium, neodymium, promethium, samarium, europium, gadolinium, terbium, dysprosium, holmium, erbium, thulium, ytterbium, and lutetium. Each of these metals is capable of exhibiting a +3 oxidation state and thus has the ability to form trivalent cations. Further advantageous properties of some of these metals (for imaging purposes, and in particular for magnetic resonance imaging) include their number of unpaired electrons, magnetic moment, and parallel spin properties.

Delivery beacons according to embodiments of the present invention, their analogs, and derivatives offer a dual imaging modality for tracking transport in vitro on the nm/μm scale by way of microscopy (using the luminescent Eu³⁺-chelated structures) and for detection with MRI within bulk cultured tissues on the sub-mm scale (using the paramagnetic Gd³⁺-chelated materials). These scaffolds offer a unique motif that can be readily tailored and optimized as both luminescent and MRI theranostic delivery agents.

Embodiments of the invention include unchelated polymers with a positive charge capable of binding with metals having repeating units comprising:

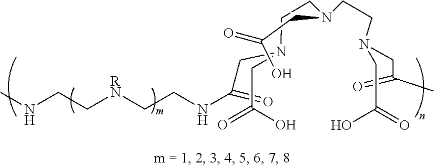

wherein R is a hydrogen atom, or a methyl or t-butoxycarbonyl (Boc) group and n is an integer ranging from 2 to 10,000,000.

Such polymers are capable of forming a zwitterionic structure in the physiological pH range which allow for aggregating phenomenon and interesting drug delivery properties.

Embodiments of the invention relate to polymeric imaging beacons comprising repeating units of metal chelates within an oligoamine backbone, the repeating units comprising:

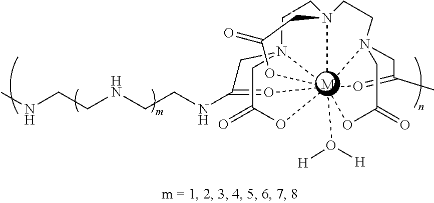

wherein n is an integer ranging from 2 to 10,000,000, such as from 2 to 1,000,000, or from 2 to 500,000, or from 2 to 250,000, or from 2 to 100,000, or from 2 to 5,000, or from 2 to 3,000, or from 2 to 1,000, and so on;

M is a metal capable of exhibiting an imaging functionality for an imaging modality; and

the oligoamine backbone comprises from 1 to 8 ethlyeneamines and is chosen from diethyleneamine, triethylenediamine, tetraethylenetriamine, pentaethylenetetraamine, hexaethylenepentamine, heptaethylenehexamine, octaethyleneheptamine, or nonethyleneoctaamine.

In preferred embodiments, the polymeric imaging beacons can comprise M, a metal chosen from copper, manganese, iron, or a lanthanide metal chosen from lanthanum, cerium, praseodymium, neodymium, promethium, samarium, europium, gadolinium, terbium, dysprosium, holmium, erbium, thulium, ytterbium, and lutetium, or any metal or other radioactive element capable of being used as an imaging agent, such as for X-ray imaging, PET imaging, SPECT imaging, MRI imaging, or even Fluorescence or Luminescence.

Preferred embodiments include such polymeric imaging beacons, wherein the repeating units comprise from 3 or 4 ethyleneamines in the oligoamine backbone, namely those comprising a tetraethylenetriamine or pentaethylenetetraamine group.

More preferred are such polymeric imaging beacons comprising a lanthanide metal, such as poly[(tetraethylenetriamine)amido(Ln³⁺)diethylenetriaminetriacetate] and poly[(pentaethylenetetramine)amido(Ln³⁺)-diethylenetriaminetriacetate].

More particularly, preferred embodiments of the polymeric imaging beacons can have a structure defined by:

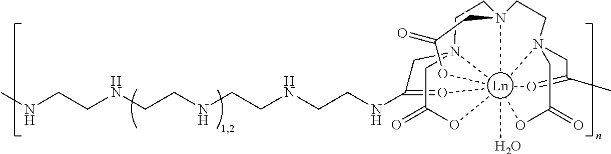

wherein n ranges from 2 to 10,000,000;

wherein Ln is a lanthanide metal chosen from lanthanum, cerium, praseodymium, neodymium, promethium, samarium, europium, gadolinium, terbium, dysprosium, holmium, erbium, thulium, ytterbium, and lutetium; and the oligoamine backbone comprises a tetraethylenetriamine or pentaethylenetetraamine to form an imaging beacon chosen from:

poly[(tetraethylenetriamine)amido(Ln³⁺)diethylenetriaminetriacetate] or

poly[(pentaethylenetetramine)amido(Ln³⁺)-diethylenetriaminetriacetate].

Especially preferred embodiment include any of the polymeric imaging beacons described in this specification, wherein Ln is gadolinium, europium, or terbium.

Polyplexes are also within the scope of the invention. Polyplex embodiments according to the invention can comprise a complex of the polymer and another molecule (such as an active biological agent, for example a drug), the polyplex comprising:

a) a polymeric portion comprising repeating units of metal chelates within an oligoamine backbone, the repeating units comprising:

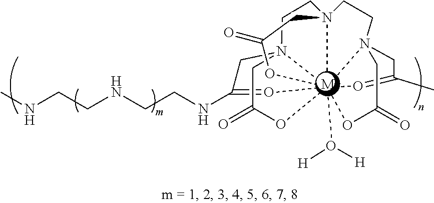

wherein n ranges from 2 to 10,000,000;

M is a metal capable of exhibiting an imaging functionality for an imaging modality; and the oligoamine backbone comprises from 1 to 8 ethlyeneamines and is chosen from diethyleneamine, triethylenediamine, tetraethylenetriamine, pentaethylenetetraamine, hexaethylenepentamine, heptaethylenehexamine, octaethyleneheptamine, or nonethyleneoctaamine; and

b) a molecule in complex (e.g., bonded or otherwise associated with) with the polymeric portion chosen from a polynucleotide (such as for example any nucleic acid), a small molecule drug, a biologic, a protein, and a peptide sequence.

Polynucleotides in the complexes can include any nucleic acid comprising from about 5 bases to about 200 kilobases. Non-limiting examples of nucleic acids include mRNA, tmRNA, tRNA, rRNA, siRNA, shRNA, PNA, ssRNA, dsRNA, ssDNA, dsDNA, DNA:RNA hybrid molecules, plasmids, artificial chromosomes, gene therapy constructs, cDNA, PCR products, restriction fragments, ribozymes, antisense constructs, and combinations thereof. Reviews of tmRNA include Muto A, Ushida C, Himeno H. A bacterial RNA that functions as both a tRNA and an mRNA, Trends Biochem Sci. 1998 January; 23(1):25-9; and Withey J H, Friedman D I, The biological roles of trans-translation, Curr Opin Microbiol. 2002 April; 5(2):154-9). The nucleic acid may comprise one or more chemical modifications.

A complex according to the invention may further comprise one or more transfection agents, one or more recombinases and, additionally or alternatively, one or more recombination proteins.

An exemplary nucleic acid used in the invention includes, in some embodiments, a sequence that encodes a protein or a portion thereof. In some embodiments, a cellular nucleic acid encoding the protein, or a portion thereof, is desirably replaced by the sequence in one form of gene therapy. Additionally or alternatively, the protein is expressed in the cell. The protein may be exogenous or endogenous. In the latter case, the cells to be transfected may comprise a non-functional form of the protein.

The polyplexes can also be delivered in the form of compositions, such as pharmaceutical compositions. In certain embodiments, the molecule in the polyplex is one or more of the nucleic acids that has a biological activity, including but not limited to therapeutic activity. By way of non-limiting example, biologically active nucleic acids are selected from the group consisting of mRNA, tmRNA, tRNA, rRNA, siRNA, shRNA, PNA, ssRNA, dsRNA, ssDNA, dsDNA, DNA:RNA hybrid molecules, plasmids, artificial chromosomes, gene therapy constructs, cDNA, PCR products, restriction fragments, ribozymes, antisense constructs, and combinations thereof.

Additionally or alternatively, a polypeptide of the complex can be biologically active. A biologically active polypeptide may be a therapeutic protein. By way of non-limiting example, bioactive proteins include antibodies or antibody fragments, hormones, enzymes, transcription factors, growth factors, and the like.

The invention further provides a method of providing gene therapy to an individual in need thereof, of treating an individual suffering from a disease or disorder, the method comprising contacting the individual, or cells therefrom, with one or more complexes, compositions and/or pharmaceutical compositions of the invention.

The invention provides for polymers for use as gene delivery agents. These polymers bind products, e.g., oligonucleotides, and facilitate cellular uptake. Embodiments of the invention provide for the in vitro or in vivo delivery of plasmid DNA into cells.

In one embodiment, the present invention relates to the use of polymers for delivering nucleic acids into a cell. In one embodiment, the nucleic acid is an oligonucleotide. In another embodiment, the oligonucleotide contains from about 10 to about 1000 nucleotides. In another embodiment, the oligonucleotide is an antisense oligonucleotide or oligodeoxynucleotide. In another embodiment, the oligonucleotide is an oligonucleotide, an antisense oligonucleotide residue or oligodeoxynucleotide residue.

In another embodiment, the nucleic acid is selected from the group consisting of antisense constructs, antisense polynucleotide, artificial chromosomes, cDNA, concatemers, concatemeric decoy oligonucleotides, CpG oligomers, cyclic oligonucleotides, decoy oligonucleotides, DNA:RNA hybrid molecules, dsDNA, dsRNA, gene therapy constructs, LNA, morpholinos, mRNA, oligonucleotides and oligodeoxynucleotides with phosphorodiester backbones or phosphorothioate backbones, PCR products, plasmids, PNA, restriction fragments, ribozyme, RNA, RNAi, RNAi inducing polynucleotide, rRNA, shRNA, siRNA, spiegelmers, ssDNA, ssRNA, tmRNA, transgenes, tricyclo-DNA, triple helices, tRNA, and combinations of any two or more thereof.

In another embodiment, the present invention provides for the use of polymers to deliver a concatemer to a cell. In another embodiment, the present invention provides for the use for the inventive polymers to deliver a concatemerized double-stranded oligonucleotide molecules (CODN) for transcription factor decoys. In one embodiment, the concatemers consist of a variable number of end-to-end repeated copies of a short (more than 5, 10, 15, 20, 2, 3035, 40, 45, 50, 75, 100, or more by but generally less than about 3 kb) dsDNA containing a sequence or sequences that act as transcription factor decoys.

The use of the concatemers provides one or more of the following benefits: a) increased half-life of the nucleotide within the cell; b) increased efficacy of each single molecule, since each contains multiple copies of the specific decoy; c) the molar amount of decoy can be titrated to achieve a specific degree of transcription factor blockade; d) CODNs can be designed to block subsets of transcription factor binding sites that may underlie biological variation in transcription factor response; e) a combinatorial blockade, since each CODN can bind multiple transcription factors, where use of concatemers allows for delivery of decoys for two or more transcription factors to be done in a precisely controlled manner. This latter point is relevant to two important issues. First, to any use requiring titration of transcription factor blockade, especially of one transcription factor relative to another. For instance, if one wishes to completely block factor X and block factor Y only 25%, this can be done by empirically determining the ratio of the decoy for X and Y required and assembling the CODN to this requirement. Second, to the fact that transcription factors often act together to activate discrete subsets of genes. For instance, NF-kB and AP-1 each act primarily on a certain subset of promoters. There is however, a common subset that requires the cooperative binding of both transcription factors to nearby sites on the promoter to properly activate gene expression. The concatemer allows blocking of these genes with relative specificity by titrating the decoys for the two transcription factors, or by designing a unique CODN to the specific combination of NF-kB and AP-1 binding sites found in the specific promoter.

The invention provides for the use of the polymers for covalent addition of targeting peptides, receptor binding peptides/protein domains and antibody fragments that may be used to target the CODN/polymer complexes to a specific cell type; thus the agent can be made organ-, tissue- and/or cell-type specific.

In another embodiment, the present invention provides for using the inventive polymers for targeting peptides and/or antibodies for specific stress and/or drug induced cellular receptors. In one embodiment, the inventive polymers target the CODN/polymer complexes to ischemic, inflamed or cancerous tissues.

In another embodiment, the present invention provides for using linker peptides containing the sequence recognized by the TNF-alpha converting enzyme (TACE) or another exopeptidase or endopeptidase in order to allow the agent to deliver the CODN/polymer complex to the cell and then cleave off the targeting peptide.

In another embodiment, the present invention provides for using the inventive polymers to deliver intact genes (transgenes), plasmids, RNAi, siRNA, morpholinos or other kinds of RNA, proteins and polynucleotides. In one embodiment, the genes incorporate tissue-specific promoters, controllable promoters, promoters that may be silenced by specific CODN/polymer combinations and may constitute two- and three-unit systems for gene expression, control and DNA transposition (i.e. insertion, excision and targeting of transgenes and other DNA molecules).

In another embodiment, the present invention provides for use of the inventive polymers in vitro or in vivo, in isolated cells or intact animals in which specific blockade of transcription factors or delivery of DNA or other biological effector is desirable. In one embodiment, this includes use as a research tool, including studies of specific genes and studies to identify specific genes regulated by the transcription factors targeted (relates to development of specific CODN/polymer complexes and related gene marker mouse lines described below). For clinical use, this would include, but is not limited to delivery of transcription factor decoys (e.g. CODNs) that block transcription factors implicated in disease, response to surgery and/or trauma, developmental defects, aging, toxic exposure, etc.

In another embodiment, the present invention provides for the delivery of one or more imaging agents for real-time and still imaging within a cell or tissue.

Yet further embodiments include methods of delivering a biologically active molecule to a cell, comprising contacting the cell with a polyplex formed from an interaction between a biologically active molecule and a cellular delivery polymer, wherein the cellular delivery polymer comprises repeating units of lanthanide (or other metal with imaging capabilities) chelates within an oligoamine backbone.

Other methods included in embodiments of the invention involve delivering one or more biologically active molecules comprising at least one nucleic acid molecule or at least one polypeptide or at least one of both to a cell by way of administering the biologically active molecule with a polymer-based delivery vehicle according to the invention. In a specific embodiment of the invention, the biologically active molecule comprises a nucleic acid. In a more specific embodiment of the invention, the nucleic acid comprises an oligonucleotide. In yet another embodiment of the invention, the nucleic acid is selected from the group consisting of mRNA, tmRNA, tRNA, rRNA, siRNA, shRNA, PNA, ssRNA, dsRNA, ssDNA, dsDNA, DNA: RNA hybrid molecules, plasmids, artificial chromosomes, gene therapy constructs, cDNA, PCR products, restriction fragments, ribozymes, antisense constructs, and combinations thereof.

Preferred polyplex embodiments include those where the repeating units comprise from 3 or 4 ethyleneamines in the oligoamine backbone, namely those comprising a tetraethylenetriamine or pentaethylenetetraamine group.

More preferred are such polyplexes or polymeric imaging beacons comprising a lanthanide metal, such as poly[(tetraethylenetriamine)amido(Ln³⁺)diethylenetriaminetriacetate] and poly[(pentaethylenetetramine)amido(Ln³⁺)-diethylenetriaminetriacetate].

Especially preferred embodiments include any of the polymeric imaging beacons, chelated or free chelates, or polyplexes described in this specification, wherein Ln is gadolinium, europium, or terbium.

Nucleic acid delivery into cells in vivo or in vitro is also within the scope of embodiments of the present invention. Even further preferred are polyplexes as described above, wherein the molecule in complex with the polymeric portion is a nucleic acid chosen from pDNA, siRNA, or an oligodeoxynucleotide.

The size and shape of the polyplexes according to the invention is not critical. For example, nanoparticle-sized complexes and polymers having an average particle size of about 1 to 5,000 nm may be preferred for certain applications. Also those having a generally spherical morphology and average particle diameters between 20 nm and 100 nm may be preferred. Even further, nanoparticle polyplexes having average particle diameters between 30 nm and 60 nm may be applicable for certain embodiments.

Methods of delivering a polynucleotide into a cell are also included within the scope of the present invention. For example, such methods can comprise:

administering in vivo or contacting with a cell in vitro a polyplex comprising:

a) a polymeric portion comprising repeating units of metal chelates within an oligoamine backbone, the repeating units comprising:

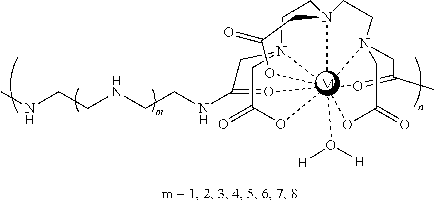

wherein n ranges from 2 to 10,000,000;

M is a metal capable of exhibiting an imaging functionality for an imaging modality; and

the oligoamine backbone comprises from 1 to 8 ethlyeneamines and is chosen from diethyleneamine, triethylenediamine, tetraethylenetriamine, pentaethylenetetraamine, hexaethylenepentamine, heptaethylenehexamine, octaethyleneheptamine, or nonethyleneoctaamine; and

b) a molecule in complex with the polymeric portion chosen from a polynucleotide, a small molecule drug, a biologic, a protein, and a peptide sequence.

Preferred method embodiments may include such methods, wherein the administering is of a polyplex, wherein the repeating units comprise:

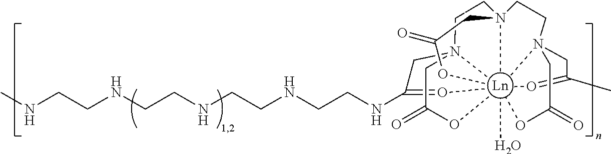

wherein n ranges from 2 to 10,000,000;

wherein Ln is a lanthanide metal chosen from lanthanum, cerium, praseodymium, neodymium, promethium, samarium, europium, gadolinium, terbium, dysprosium, holmium, erbium, thulium, ytterbium, and lutetium; and the oligoamine backbone comprises a tetraethylenetriamine or pentaethylenetetraamine to form an imaging beacon chosen from:

poly[(tetraethylenetriamine)amido(Ln³⁺)diethylenetriaminetriacetate] or

poly[(pentaethylenetetramine)amido(Ln³⁺)-diethylenetriaminetriacetate].

Even further, such methods described in this specification can involve targeting (by way of administering the complexes to an animal in vivo or to cells or tissue in vitro) a selected number of cells, and wherein the polynucleotide is chosen from pDNA, siRNA, or an oligodeoxynucleotide and wherein uptake by a desired portion of the cells is accomplished.

Preferred embodiments may include methods further comprising visualizing by luminescence the polyplex or part thereof by microscopy for tracking intracellular delivery of the polyplex. Especially preferred are visualization techniques involving employing Eu3+, Tb3+, or Sm3+ as the lanthanide which enables luminescence of the polyplex and visualization by microscopy. If desired, certain applications can further comprise imaging the polyplex or part thereof by magnetic resonance imaging (MRI) for following polynucleotide delivery within cultured cells or tissues, or in vivo. Especially preferred are such methods wherein Gd3+ is the lanthanide which enables imaging of the polyplex by MRI.

Methods described in this specification can involve administering polyplexes to target a selected number of cells, and wherein the nucleic acid is pDNA or siRNA, and wherein uptake is accomplished by 90-100% of the cells affected. Any amount of uptake can be exhibited, including from 20-100% of the cells targeted, but preferred are situations where 50-100%, or 60-80%, or 70-95% of the cells targeted involve uptake of the polyplexes, and especially preferred are situations where 98-100% of the cells succeed in accepting the polyplexes into the cell. Preferred methods are those where a majority of the nucleic acid entering the cells is found in the cytoplasm and less is found in the nucleus of the target cells.

Compositions and pharmaceuticals comprising the imaging beacons according to embodiments of the invention and/or the nanoparticle polyplexes thereof are also encompassed by this disclosure. Any of the beacons polyplexes, in part or whole, or even combinations thereof can be formulated into compositions or pharmaceuticals comprising a desired amount or type of polynucleotide (such as a nucleic acid), any drug (such as a small molecule drug), any biologic, any peptide, or any protein to be delivered to the cells. Preparations can be formulated for animals, including humans according to generally known and accepted procedures.

Also included in embodiments of the invention is the use of the imaging beacons and polyplexes described in this specification in the manufacture of a medicament for delivering compounds into cells, including nucleic acids, and for tracking such delivery by imaging.

Polyamides For Nucleic Acid Delivery. In the description that follows, a number of terms used in molecular biology and medical/pharmaceutical sciences are utilized extensively. In order to provide a clear and consistent understanding of the specification and claims, including the scope to be given such terms, the following definitions are provided. Under these definitions, the following terms have the following meaning unless otherwise specified herein:

Association: the covalent or non-covalent joining of two or more molecules, which may occur permanently, temporary, or transiently. A molecular complex is formed by the stable or semi-stable association of two or more compounds.

Base Pair (bp): a partnership of adenine (A) with thymine (T), or of cytosine (C) with guanine (G) in a double stranded DNA molecule. In RNA, uracil (U) is substituted for thymine. Base pairs are the to be “complementary” when their component bases pair up normally when a DNA or RNA molecule adopts a double stranded configuration.

The term “biologically active molecule” as used herein, refers to compounds or molecules that are capable of eliciting or modifying a biological response in a system. Non-limiting examples of biologically active siNA molecules either alone or in combination with other molecules contemplated by the instant invention include therapeutically active molecules such as antibodies, cholesterol, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siNA, dsRNA, allozymes, aptamers, decoys and analogs thereof. Biologically active molecules of the invention also include molecules capable, of modulating the pharmacokinetics and/or pharmacodynamics of other biologically active molecules, for example, lipids and polymers such as polyamines, polyamides, polyethylene glycol and other polyethers.

Cellular Delivery (also referred to herein interchangeably and equivalently as “delivery”): a process by which a desired compound is transferred to a target cell such that the desired compound is ultimately located inside the target cell, or in or on the target cell membrane. In certain uses delivery to a specific target cell type is preferable.

Cellular Delivery Molecule: a molecule that mediates the Cellular Delivery of itself, a molecular complex comprising the Cellular Delivery Molecule, and/or a molecule comprising the Cellular Delivery Molecule.

Cell delivery polymer: a polymer that functions as a Cellular Delivery Molecule, either by itself, as a part of a molecular complex.

Complementary Nucleotide Sequence: a sequence of nucleotides in a single-stranded molecule of DNA or RNA that is sufficiently complementary to another single strand to specifically (non-randomly) hybridize to it with consequent hydrogen bonding.

Construct: a vector sequence, or a portion thereof, that has been linked with one or more non-vector sequences.

Inducer: a molecule that triggers gene transcription by binding to a regulator protein such as a repressor. Induction: the switching on of transcription as a result of interaction of an inducer with a positive or negative regulator.

Negative Regulation of Transcription: a mechanism of gene expression control where a gene is transcribed unless prevented by the action of a negative regulator, or repressor.

Nucleotide: a base-sugar-phosphate combination. Nucleotides are monomeric units of a nucleic acid sequence (DNA and RNA). Nucleotides may also include mono-, di- and triphosphate forms of such nucleotides. The term nucleotide includes ribonucleoside triphosphates ATP, UTP, ITP, CTG, GTP and deoxyribonucleoside triphosphates such as dATP, dCTP, dITP, dUTP, dGTP, dTTP, or derivatives thereof. Such derivatives include, for example, [aS]dATP, 7-deaza-dGTP and 7-deaza-dATP, and nucleotide derivatives that confer nuclease resistance on the nucleic acid molecule containing them. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. According to the present invention, a “nucleotide” may be unlabeled or detectably labeled by well known techniques. Detectable labels include, for example, radioactive isotopes, fluorescent labels, chemiluminescent labels, bioluminescent labels and enzyme labels. Various labeling methods known in the art can be employed in the practice of this invention.

Nucleotide Analog: a purine or pyrimidine nucleotide that differs structurally from an A, T, G, C, or U base, but is sufficiently similar to substitute for the normal nucleotide in a nucleic acid molecule. Inosine (I) is a nucleotide analog that can hydrogen bond with any of the other nucleotides, A, T, G, C, or U. In addition, methylated bases are known that can participate in nucleic acid hybridization. Methods of preparing and using modified oligonucleotides are described in: Verma S, Eckstein F. Modified oligonucleotides: synthesis and strategy for users, Annu Rev Biochem. 1998; 67:99-134.

By way of non-limiting example, nucleotide analogs include 2,6-diamino purine, 6-methyladenine, 8-azaguanine, 5-bromouracil, 5-hydroxymethyl uracil, 5-methylcytosine (5MC), 5-hydroxymethylcytosine (HMC), 8-chloroadenosine, glycosyl HMC, and gentobiosyl HMC. Fluorescent nucleotide analogs, such as those described by Jameson and Eccleston (Fluorescent nucleotide analogs: synthesis and applications, Methods Enzymol. 1997; 278:363-90), and cyclic nucleotide analogs, such as those described by Schwede et al. (Cyclic nucleotide analogs as biochemical tools and prospective drugs, Pharmacol Ther 2000 87(2-3):199-226) may also be used in the invention.

Nucleic Acid: As used herein “nucleic acid” and its grammatical equivalents will include the full range of polymers of single or double stranded nucleotides. A nucleic acid typically refers to a polynucleotide molecule comprised of a linear strand of two or more nucleotides (deoxyribonucleotides and/or ribonucleotides) or variants, derivatives and/or analogs thereof. The exact size will depend on many factors, which in turn depends on the ultimate conditions of use, as is well known in the art. The nucleic acids of the present invention include without limitation primers, probes, oligonucleotides, vectors, constructs, plasmids, genes, transgenes, genomic DNA, cDNA, PCR products, restriction fragments, and the like.

Promoter: As used herein, a promoter is an example of a transcriptional regulatory sequence, and specifically is a DNA sequence generally described as the 5′-region of a gene located proximal to the start codon. The transcription of an adjacent DNA segment is initiated at the promoter region. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

Recognition sequence: As used herein, a recognition sequence is a particular sequence to which a protein, chemical compound, DNA, or RNA molecule (e.g., restriction endonuclease, a modification methylase, or a recombinase) recognizes and binds. In the present invention, a recognition sequence will typically, but need not, refer to a recombination site. For example, the recognition sequence for Cre recombinase is loxP which is a 34 base pair sequence comprised of two 13 base pair inverted repeats (serving as the recombinase binding sites) flanking an 8 base pair core sequence. See FIG. 1 of Sauer, B., Current Opinion in Biotechnology 5:521-527 (1994). Other examples of recognition sequences are the attB, attP, attL, and attR sequences which are recognized by the recombinase enzyme Integrase. The attB site is an approximately 25 base pair sequence containing two 9 base pair core-type Int binding sites and a 7 base pair overlap region. The attP site is an approximately 240 base pair sequence containing core-type Int binding sites and arm-type Int binding sites as well as sites for the auxiliary proteins integration host factor (IHF), FIS and excisionase (Xis). See Landy, Current Opinion in Biotechnology 3:699-707 (1993). Such sites may also be engineered according to the present invention to enhance production of products in the methods of the invention. When such engineered sites lack the P1 or H1 domains to make the recombination reactions irreversible (e.g., attR or attP), such sites may be designated attR′ or attP′ to indicate that the domains of these sites have been modified in some way.

Repressor: protein preventing transcription by binding to a specific site on DNA.

Target Cell: any cell to which a desired compound is delivered. Cells to which the delivery methods of this invention can be applied include cells in vitro, cells ex vivo or cells in vivo. Target cells may be in cell culture, on tissue culture, in any form of immobilized state, or grown on liquid, semi-solid or solid medium. Target cells may be in the form of a monolayer. Target cells may be collected from an organism and/or cultured by any known method. Target cells include cells without cell walls and cells from which cell walls have been removed by any known treatment (e.g., formation of protoplasts) from which viable cells can be recovered.

Transcriptional regulatory sequence: As used herein, transcriptional regulatory sequence is a functional stretch of nucleotides contained on a nucleic acid molecule, in any configuration or geometry, that acts to regulate the transcription of one or more structural genes into messenger RNA. Examples of transcriptional regulatory sequences include, but are not limited to, promoters, enhancers, repressors, and the like. “Transcription regulatory sequence,” “transcription sites” and “transcription signals” may be used interchangeably.

Transfection: the delivery of expressible nucleic acid to a target cell, such that the target cell is rendered capable of expressing the nucleic acid. It will be understood that the term “nucleic acid” includes both DNA and RNA without regard to molecular weight, and the term “expression” means any manifestation of the functional presence of the nucleic acid within the cell including, without limitation, both transient expression and stable expression.

Transfection Agent: substance which provides significant enhancement of transfection (2-fold or more) over compositions that do not comprise the transfection agent.

Vector: As used herein, a vector is a nucleic acid molecule that provides a useful biological or biochemical property to a nucleic acid sequence or molecule of interest, for example, an Insert, a coding region, etc. Examples include plasmids, phages, autonomously replicating sequences (ARS), centromeres, and other nucleic acid sequences that are able to replicate or be replicated in vitro or in a host cell, or to convey a desired nucleic acid segment to a desired location within a host cell. A vector may comprise various structural and/or functional sequences, for example, one or more restriction endonuclease recognition sites at which the vector sequences can be manipulated in a determinable fashion without loss of an essential biological function of the vector, and into which a nucleic acid fragment can be inserted, for example to bring about its replication and/or cloning. Vectors can further provide primer sites, e.g., for PCR, transcriptional and/or translational initiation and/or regulation sites, recombinational signals, replicons, selectable markers, and other sequences known to those skilled in the art. A vector comprising a nucleic acid insert is a Construct. Thus, a gene therapy construct is a gene therapy vector into which a therapeutic gene has been cloned. Similarly, a construct that expresses an antisense transcript is an “antisense construct.”

Biologically Active: As used herein, the term “biologically active” (synonymous with “bioactive”) indicates that a composition or compound itself has a biological effect, or that it modifies, causes, promotes, enhances, blocks, or reduces a biological effect, or which limits the production or activity of, reacts with and/or binds to a second molecule that has a biological effect. The second molecule can, but need not, be endogenous.

A “biological effect” may be but is not limited to one that stimulates or causes an immunoreactive response; one that impacts a biological process in a cell, tissue or organism (e.g., in an animal); one that impacts a biological process in a pathogen or parasite; one that generates or causes to be generated a detectable signal; and the like. Biologically active compositions, complexes or compounds may be used in investigative, therapeutic, prophylactic and diagnostic methods and compositions. Biologically active compositions, complexes or compounds act to cause or stimulate a desired effect upon a cell, tissue, organ or organism (e.g., an animal). Non-limiting examples of desired effects include modulating, inhibiting or enhancing gene expression in a cell, tissue, organ, or organism; preventing, treating or curing a disease or condition in an animal suffering therefrom; limiting the growth of or killing a pathogen in an animal infected thereby; augmenting the phenotype or genotype of an animal; stimulating a prophylactic immunoreactive response in an animal; or diagnosing a disease or disorder in an animal.

In the context of investigative applications of the invention, including but not limited to forensic and scientific research applications, the term “biologically active” indicates that the composition, complex or compound has an activity that results, directly or indirectly, in a change in some form of measurable output in materials, biological samples, cells or organisms that have been contacted therewith. Investigative applications may be used to determine the quantity or concentration of a selected target compound in a test sample, to determine the effect of a bioactive compound upon cells or animals, or to screen for compounds having an activity that alters, blocks or augments a selected biological activity.

In the context of therapeutic applications of the invention, the term “biologically active” indicates that the composition, complex or compound has an activity that impacts an animal suffering from a disease or disorder in a positive sense and/or impacts a pathogen or parasite in a negative sense. Thus, a biologically active composition, complex or compound may cause or promote a biological or biochemical activity within an animal that is detrimental to the growth and/or maintenance of a pathogen or parasites; or of cells, tissues or organs of an animal that have abnormal growth or biochemical characteristics, such as cancer cells.

In the context of prophylactic applications of the invention, the term “biologically active” indicates that the composition or compound induces or stimulates an immunoreactive response. In some preferred embodiments, the immunoreactive response is designed to be prophylactic, i.e., to prevent infection by a pathogen. In other preferred embodiments, the immunoreactive response is designed to cause the immune system of an animal to react to the detriment of cells of an animal, such as cancer cells, that have abnormal growth or biochemical characteristics. In this application of the invention, compositions, complexes or compounds comprising antigens are formulated as a vaccine.

In the context of diagnostic applications on the invention, the term “biologically active” indicates that the composition, complex or compound can be used for in vivo or ex vivo diagnostic methods and in diagnostic compositions and kits. For diagnostic purposes, a preferred biologically active composition or compound is one that can be detected, typically (but not necessarily) by virtue of comprising a detectable polypeptide. Antibodies to an epitope found on composition or compound may also be used for its detection. It will be understood by those skilled in the art that a given composition, complex or compound may be biologically active in therapeutic, diagnostic and/or prophylactic applications. A composition, complex or compound that is described as being “biologically active in a cell” is one that has biological activity in vitro (i.e., in a cell or tissue culture) or in vivo (i.e., in the cells of an animal). A “biologically active component” of a composition or compound is a portion thereof that is biologically active once is liberated from the composition or compound. It should be noted that such a component may also be biologically active as a moiety or other portion of the composition or compound.

In the disclosure and the claims, “and/or” means additionally or alternatively. Moreover, any use of a term in the singular also encompasses plural forms.

Other terms used in the fields of recombinant DNA technology, molecular and cell biology, and the medical/pharmaceutical arts, as used herein, are intended to encompass the broadest scope term understood in the art for a given and will be generally understood by one of ordinary skill in the applicable arts.

In one embodiment, the invention encompasses a method of delivering a biologically active molecule to a cell, comprising contacting the cell with (a) a biologically active molecule and (b) a cellular delivery polymer.

In one embodiment, the present invention also provides for compositions and non-covalent complexes comprising one or more polymers of the present invention and at least one nucleic acid molecule (e.g., one or more oligonucleotides) or at least one polypeptide or both. The invention also provides compositions comprising such complexes.

Complexes according to the invention or portions thereof, can comprise a cellular delivery molecule or agent that can facilitate the translocation of the complex or portion thereof into cells. In some embodiments, cellular delivery molecules for use in the present invention may comprise one or more one or more polymers of the present invention.

In some embodiments, a cell, tissue, organ or organism may be contacted with a complex of the invention. Preferably, the complex is taken up by the cell or by one or more cells of the tissue, organ or organism.

In another exemplary and non-limiting embodiment of the invention, compositions comprising complexes between cellular delivery polymers and oligonucleotides are formed and can be applied to cultured mammalian cells. The complex may also comprise a combination of labeled and nonlabeled nucleic acid and or peptide. These complexes allow mediation of an activity associated with the oligonucleotide, which, by way of non-limiting example, can be a gene-containing oligonucleotide, an antisense oligonucleotide, an aptamer, a short interfering RNA (siRNA), a short hairpin RNA (shRNA), a small temporally regulated RNA (stRNA), and the like. In some embodiments, oligonucleotides are preferred.

In other specific embodiments, the biologically active molecule and/or cell delivery agent is covalently labeled with fluorophores (fluorescent moiety), for example with fluorescein or a derivative of fluorescein.

In another embodiment, the compositions may comprise one or more fluorescent molecules or moieties, which may be the same or different, and may be covalently attached to one or more polypeptides and/or nucleic acid molecules in the complexes of the invention. Alternatively, or in addition, complexes of the invention may comprise one or more “free” fluorescent molecule (i.e., one or more fluorescent molecules that are not covalently attached to either the polypeptide or the oligonucleotide but may still be associated with the complex). One or more of the compounds of the compositions or complexes can be a biologically active molecule.

Kits according to the invention may comprise one or more transfection agents, one or more cells, one or more nucleic acids, one or more set of instructions, and one or more biologically active molecules. For example, embodiments of the invention include kits comprising one or more polymeric imaging beacon comprising repeating units of metal chelates within an oligoamine backbone, the repeating units comprising:

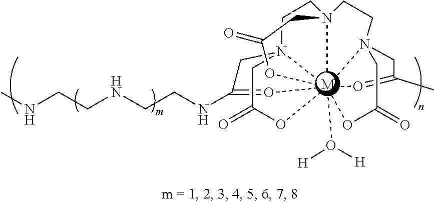

wherein n ranges from 2 to 10,000,000;

M is a metal capable of exhibiting an imaging functionality for an imaging modality; and

the oligoamine backbone comprises from 1 to 8 ethlyeneamines and is chosen from diethyleneamine, triethylenediamine, tetraethylenetriamine, pentaethylenetetraamine, hexaethylenepentamine, heptaethylenehexamine, octaethyleneheptamine, or nonethyleneoctaamine; and

and comprising one or more biologically active molecules capable of forming a complex with the beacon.

Kit components can include without limitation: additional nucleic acids, such as oligonucleotides, iRNA molecules, plasmids, etc.; one or more recombinases, including without limitation site-specific recombinases; one or more recombination proteins; and/or one or more cells. In some embodiments, the cells are competent for transfection or transformation.

In other embodiments, the invention provides a complex comprising a cell delivery polymer and a biologically active agent that is desirably taken up by cells, wherein the cell delivery polymer or biologically active agent comprises a fluorescent moiety.

The nucleic acid of the complexes and other embodiments of the invention can comprise from 5 bases to about 200 kilobases. Any type of nucleic acid may be used, including by way of non-limiting example mRNA, tmRNA, tRNA, rRNA, siRNA, shRNA, PNA, ssRNA, dsRNA, ssDNA, dsDNA, DNA:RNA hybrid molecules, plasmids, artificial chromosomes, gene therapy constructs, cDNA, PCR products, restriction fragments, ribozymes, antisense constructs, and combinations thereof. Reviews of tmRNA include Muto A, Ushida C, Himeno H. A bacterial RNA that functions as both a tRNA and an mRNA. Trends Biochem Sci. 1998 January; 23(1):25-9; and Withey J H, Friedman D I, The biological roles of trans-translation. Curr Opin Microbiol. 2002 April; 5(2):154-9). The nucleic acid may comprise one or more chemical modifications.

A complex according to the invention may further comprise one or more transfection agents, one or more recombinases and, additionally or alternatively, one or more recombination proteins.

A nucleic acid used in the invention includes, in some embodiments, a sequence that encodes a protein or a portion thereof. In some embodiments, a cellular nucleic acid encoding the protein, or a portion thereof, is desirably replaced by the sequence in one form of gene therapy. Additionally or alternatively, the protein is expressed in the cell. The protein may be exogenous or endogenous. In the latter case, the cells to be transfected may comprise a non-functional form of the protein.

A composition of the invention may be a pharmaceutical composition. In certain embodiments, the biologically active molecule is one or more of the nucleic acids that has a biological activity, including but not limited to therapeutic activity. By way of non-limiting example, biologically active nucleic acids are selected from the group consisting of mRNA, tmRNA, tRNA, rRNA, siRNA, shRNA, PNA, ssRNA, dsRNA, ssDNA, dsDNA, DNA:RNA hybrid molecules, plasmids, artificial chromosomes, gene therapy constructs, cDNA, PCR products, restriction fragments, ribozymes, antisense constructs, and combinations thereof.

Additionally or alternatively, polypeptide of the complex is biologically active. A biologically active polypeptide may be a therapeutic protein. By way of non-limiting example, bioactive proteins include antibodies or antibody fragments, hormones, enzymes, transcription factors, growth factors, and the like.

The invention further provides a method of providing gene therapy to an individual in need thereof, of treating an individual suffering from a disease or disorder, the method comprising contacting the individual, or cells therefrom, with one or more complexes, compositions and/or pharmaceutical compositions of the invention.

The invention further provides a method of testing a cellular response to a test compound, the method comprising: (a) contacting a first cell with, in any order or combination, a biologically active molecule and a cellular delivery polymer; (b) contacting a second cell with, in any order or combination, a second biologically active molecule and the cellular delivery polymer; (c) contacting the cells with the test compound, before (a); during (a) or (b); between (a) and (b); and, additionally or alternatively, after (b); (d) measuring and comparing at least one parameter of from the first cell with the signal from the second cell. In certain embodiments, one or more of the cells comprise one or more reporter genes that generate a detectable signal or interfere with the production of a detectable signal.

In one specific embodiment, the present invention provides polymer compositions, complexes and methods for delivering one or more nucleic acids (e.g., one or more nucleic acid molecules, oligonucleotides, polynucleotides, vectors, genes and the like) and/or one or more peptides (e.g., one or more peptides, oligopeptides, polypeptides, proteins or protein complexes) to cells, tissues, organs and whole organisms. The compositions and complexes of the invention typically comprise one or more nucleic acids and/or one or more proteins or polypeptides (which can be cellular delivery (suitably, translocating) peptides, polypeptides or proteins.

In certain such aspects of the invention, the complexes comprising one or more nucleic acids and/or one or more peptides are delivered to and taken up by the cells, tissues, organs or organisms, and cells, tissues, organs or organisms. The invention also provides compositions comprising the polymer complexes of the invention and one or more additional components. Suitable such compositions, for example, include pharmaceutical compositions comprising one or more of the complexes of the invention and one or more pharmaceutically acceptable carriers, excipients or diluents therefor.

The invention also provides methods for producing such complexes and compositions, and methods of using such complexes and compositions to deliver one or more nucleic acid molecules and/or one or more peptides to cells, tissues, organs or organisms, for example for therapeutic or prophylactic purposes.

The invention also provides kits comprising the complexes and compositions of the invention, and optionally further comprising one or more additional components suitable for use in or with the complexes and compositions, and/or for carrying out the methods, of the present invention.

The present invention provides a new class of non-viral transduction vectors that can be used for both in vivo and in vitro applications. In particular, these vectors can be used for gene transfer applications. These new gene transduction vectors can achieve transfer efficiencies far greater to commercially available polymeric and liposomal gene transfer vectors while maintaining little or no toxicity in vitro. Their low in vitro toxicity makes them ideal candidates for in vivo use. The present invention provides a gene transfer vector that has comparable efficiency to a viral vector without the potential for a life-threatening immune response.

Furthermore, the unique polycationic structure of these polymers associates with many suitable bioactive molecules, including proteins and other compounds that possess multiple cationic sites. The polymer can act as a delivery vehicle for the associated bioactive molecule, in vivo or in vitro, to the cells of interest for the bioactive molecule. The delivery action and utility of these polymers are similar to other previously described polymer-based delivery vehicles, such as for example those described in U.S. Published Patent Application Nos. 2009/0105115, 2009/0124534, and 2009/0203131, the disclosures of which are hereby incorporated by reference in their entireties.

Polypeptides. As noted above, the compositions and complexes of the present invention comprise one or more peptides, polypeptides or proteins. In certain aspects of the invention, the peptides, polypeptides or proteins used in these complexes and compositions are peptides, polypeptides or proteins that are to be delivered to cells, tissues, organs or organisms for any suitable biological, therapeutic and/or prophylactic purpose.

As used herein, the term “polypeptide” includes without limitation peptides (oligopeptides), proteins, and polypeptides. All of these are polymers of two or more amino acids joined by an amino bond. Generally, peptides comprise from 2 to about a amino acid residues, wherein “a” is any whole integer between 5 and 50, preferably between 10 and 30, and may be isolated from natural sources or more typically are synthesized in vitro. As used herein, the term “oligopeptide” may be used interchangeably and equivalently with the term “peptide” as defined above. As used herein, “polypeptides” generally comprise about b amino acids, wherein “b” is any whole integer between 25 and 50,000, preferably between 50 and 10,000, and more preferably between 50 and 1,000. The term “protein” encompasses polypeptides, as well as complexes of two or more covalently or non-covalently bonded polypeptides. Polypeptides and proteins are purified from their natural sources and/or are synthesized using recombinant DNA technology.

Peptides, polypeptides, proteins and protein complexes suitable for use in the complexes, compositions and methods of the present invention include any peptide, polypeptide, protein and protein complex, or portion thereof, that has a desired biological or physiological effect on the cells, tissues, organs and organisms to which the peptides, polypeptides, proteins and protein complexes are delivered. Non-limiting examples of such peptides, polypeptides, proteins and protein complexes include: enzymes, e.g., kinases; peptidases/proteinases; oxidoreductases; nucleases; recombinases (including Cre, Int, Flp, Tn5 resolvase, and the like); ligases (including DNA ligases and the like); lyases; isomerases (including topoisomerases and the like); polymerases (including DNA polymerases, RNA polymerases, reverse transcriptases, and the like); transferases (including terminal transferases, glutathione S-transferases, and the like); ATPases; GTPases; etc.; cytokines, e.g., growth factors (such as epidermal growth factor (EGF), fibroblast growth factors (FGFs), keratinocyte growth factors (KGFs), hepatocyte growth factors (HGFs), platelet-derived growth factor (PDGF), transforming growth factors alpha and beta (TGF-.alpha. and TGF-.beta.), neurotrophic factor (NTF), ciliary neurotrophic factor (CNTF), brain-derived neurotrophic factor (BDNTF), glial-derived neurotrophic factor (GDNTF), bone morphogenic proteins (BMPs), and the like, and variants thereof); interleukins (such as IL-1 through IL-18, and the like, and variants thereof); interferons (such as IFN-.alpha., IFN-.beta., IFN-.gamma., and the like, and variants thereof); colony-stimulating factors (such as granulocyte colony-stimulating factor (G-CSF), macrophage colony-stimulating factor (M-CSF), granulocyte-macrophase colony-stimulating factor (GM-CSF); erythropoietin (Epo); thrombopoietin (Tpo); leukemia inhibitory factor (LIF/Steel Factor); tumor-necrosis factors (TNFs); and the like, and variants thereof); peptide hormones (such as antidiuretic hormone, chorionic gonadotropin, leutenizing hormone, follicle-stimulating hormone, insulin, prolactin, somatomedins, growth hormone, thyroid-stimulating hormone, placental lactogen, and the like, and variants thereof); etc.; intraceullar signalling peptides; receptors (e.g., cytokine receptors, hormone receptors, antibody receptors, integrins and other extracellular matrix receptors, neurotransmitter receptors, viral receptors, and the like, and variants thereof); antibodies (e.g., polyclonal or monoclonal antibodies, fragments thereof (including Fab and Fc fragments and portions thereof), and multi-antibody complexes); vaccine components (including, but not limited to, proteins or peptides of etiologic agents such as viruses, bacteria, fungi (including yeasts), parasites and the like; proteins or peptides of tumor cells or other cancer-related proteins or peptides; and other proteins or peptides against which it is desirable to produce an immune response in an animal, suitably a mammal such as a human); structural and/or functional proteins or peptides (e.g., hemoglobin, albumins including serum albumins, cytoskeletal proteins, transmembrane channel proteins or peptides, and the like, and fragments or variants thereof); synthetic peptides (e.g., hexahistidine, polylysine, and other synthetic peptides of any length containing a desired sequence of two or more amino acids linked together by peptide bonds to form a peptide, oligopeptide, polypeptide or protein, any and all of which can be produced by art-known methods of synthetic peptide synthesis that will be familiar to the ordinarily skilled artisan, and that are described herein); and the like. Of course, other suitable peptides, oligopeptides, polypeptides and proteins suitable for use in accordance with the present invention (i.e., in the complexes, compositions and methods of the invention) will be familiar to one of ordinary skill and therefore are encompassed by the present invention.

Amino Acids. The term “amino acid” as used herein refers generally to a molecule having both a carboxyl (—COOH) and an amino (—NH₂) group attached to the same carbon atom, called the alpha-carbon atom. Amino acids can be represented by the general formula R—CH(NH₂)COOH, wherein R is a side chain or residue which may or may not occur naturally. Generally, the side chain (R) of an amino acid contains c carbon atoms, d nitrogen atoms, 0, 1 or 2 sulfur atoms, d oxygens, and/or d halogen atoms, wherein “c” is any whole integer from 0 to about 20, and “d” is any whole integer from 0 to about 5.

The terms “natural amino acid” and “naturally-occurring amino acid” refer to Ala, Asp, Cys, Glu, Phe, Gly, H is, Ile, Lys, Leu, Met, Asn, Pro, Gln, Arg, Ser, Thr, Val, Trp, and Tyr. “Unnatural amino acids” (i.e., amino acids do not occur naturally) include, by way of non-limiting example, homoserine, homoarginine, citrulline, phenylglycine, taurine, iodotyrosine, seleno-cysteine, norleucine (“Nle”), norvaline (“Nva”), beta-Alanine, L- or D-naphthalanine, ornithine (“Orn”), and the like.

Amino acids also include the D-forms of natural and unnatural amino acids. “D-” designates an amino acid having the “D” (dextrorotary) configuration, as opposed to the configuration in the naturally occurring (“L-”) amino acids. Where no specific configuration is indicated, one skilled in the art would understand the amino acid to be an L-amino acid. The amino acids can, however, also be in racemic mixtures of the D- and L-configuration. Natural and unnatural amino acids can be purchased commercially (Sigma Chemical Co.; Advanced Chemtech) or synthesized using methods known in the art. Amino acid substitutions may be made on the basis of similarity in polarity, charge, solubility, hydrophobicity, hydrophilicity, and/or the amphipathic nature of the residues as long as their biological activity is retained.

Peptide Synthesis. Peptides used in accordance with the present invention may be produced by a variety of methods that will be familiar to those of ordinary skill in the art. For reviews and enabling disclosures of peptide synthesis, see M. Bodanzsky, “Principles of Peptide Synthesis,” 1st and 2nd revised ed., Springer-Verlag, New York, N.Y., 1984 and 1993; Stewart and Young, “Solid Phase Peptide Synthesis,” 2nd ed., Pierce Chemical Co., Rockford, III., 1984; Fox J E. Multiple peptide synthesis. Mol. Biotechnol. 3:249-258, 1995; Kiso Y, Fujii N, Yajima H. New disulfide bond-forming reactions for peptide and protein synthesis. Braz J Med Biol Res. 27:2733-2744, 1994; Bongers J, Heimer E P. Recent applications of enzymatic peptide synthesis. Peptides. 15:183-193, 1994; Wade J D, Tregear G W. Solid phase peptide synthesis: recent advances and applications. Australas Biotechnol. 3:332-336, 1993; Fields G B, Noble R L. Solid phase peptide synthesis utilizing 9-fluorenylmethoxycarbonyl amino acids. Int J Pept Protein Res. 35:161-214, 1990; Newton R, Fox J E. Automation of peptide synthesis. Adv Biotechnol Processes. 10:1-24, 1988; Barany G, Kneib-Cordonier N, Mullen D G. Solid-phase peptide synthesis: a silver anniversary report. Int J Pept Protein Res. 30:705-739, 1987; Bodanszky M. In search of new methods in peptide synthesis. A review of the last three decades. Int J Pept Protein Res. 25:449-474, 1985; Chaiken I M. Semisynthetic peptides and proteins. CRC Crit. Rev Biochem. 11:255-301, 1981; Fridkin M, Patchomnik A. Peptide synthesis. Annu Rev Biochem. 43:419-443, 1974; Merrifield R B. Solid-phase peptide synthesis. Adv Enzymol Relat Areas Mol. Biol. 32:221-296, 1969; and U.S. Pat. No. 4,748,002 (Semi-automatic, solid-phase peptide multi-synthesizer and process for the production of synthetic peptides by the use of the multi-synthesizer) to Neimark et al.

Fusion Proteins. In certain embodiments, the peptides, polypeptides or proteins used in the present invention are in the form of fusion proteins. As used herein, the term “fusion protein” refers to a peptide, polypeptide or protein comprising a series of contiguous amino acids from one peptide, polypeptide or protein that are linked via peptide bonds to a series of contiguous amino acids from one or more additional peptides, polypeptides or proteins. For example, fusion of the glutathione S-transferase (GST) domain to a peptide, polypeptide or protein of interest allows the fusion protein to be purified by affinity chromatography on glutathione agarose (Pharmacia, Inc., 1995 catalog). The fusion protein may include one or more accessory sequences which function for detection, purification or cleavage of the fusion protein. If the peptide, polypeptide or protein of interest is fused to a series of consecutive histidines (for example 6×His), the fusion protein can be purified by affinity chromatography on chelating resins containing metal ions (Qiagen, Inc.). Fusion proteins may include sequences which function as a protein tag, such as an antibody epitope (e.g., derived from Myc), a thiorescent peptide or a poly Histag. Tags and other elements may function in the purification and/or detection of the fusion protein. In producing fusion proteins according to this aspect of the invention, it is often desirable to compare amino terminal and carboxy terminal fusions for activity, solubility, stability, and the like.

Targeting sequences are another type of accessory element that can be comprised in a fusion protein. Cellular targeting elements, which direct fusion proteins to specific cell types, include such things as antibody fragments directed to a cellular surface molecule, fragments of ligands for receptors present on a cell, cell-specific targeting sequences derived from pathogens, derivatives of cellular adhesion molecules, and the like. Intracellular targeting elements, which direct fusion proteins to subcellular locations including, without limitation, the nucleus, the cell membrane, the chloroplast, the mitochondrion, the endoplasmic reticulum, the cytoplasm, and membranes or intermembrane spaces of any of the preceding, are known and are commercially available (e.g., Invitrogen's line of pShooter vectors). Various targeting sequences are known in the art and can be readily incorporated into fusion proteins using methods known in the art. Polynucleotides encoding fusion proteins may be constructed by standard molecular biology techniques (J. Sambrook, E. F. Fritsch and T. Maniatis (1989). Molecular Cloning, A Laboratory Manual. Cold Spring Harbor Laboratory Press. Cold Spring Harbor, N.Y.).

DNA-Binding Peptides and Proteins. A variety of DNA-binding proteins, particularly those that are basic, more particularly DNA-binding proteins with a relatively high percentage of Lysine and Arginine residues (“Arg- and Lys-rich proteins”), can be used with the compositions of the invention. A DNA-binding protein can be sequence-specific, partially sequence specific, or non-specific.

See U.S. Pat. No. 5,354,844 (Protein-polycation conjugates) to Beug, et al.; U.S. Pat. No. 5,972,900 (Delivery of nucleic acid to cells) to Ferkol, Jr., et al.; U.S. Pat. No. 5,166,320 (Carrier system and method for the introduction of genes into mammalian cells); and U.S. Pat. Nos. 6,008,336, 5,844,107 and 5,877,302 (Compacted nucleic acids and their delivery to cells), U.S. Pat. No. 6,077,835 (Delivery of compacted nucleic acid to cells), all to Hanson, et al. U.S. Pat. No. 6,333,396 to Filpula, et al. (Method for targeted delivery of nucleic acids) describes a single-chain antigen-binding polypeptide comprising, at its C-terminus, N-terminus, or both, basic amino acid residues selected from the group consisting of oligo-Lys, oligo-Arg and combinations thereof. U.S. Pat. No. 6,281,005 (Automated nucleic acid compaction device) to Hanson, et al. describes a device that can be used to prepare compacted DNA complexes.

Non-Eukaryotic Histonelike Proteins. A class of DNA-binding, Arg- and Lys-rich proteins that can be used in the invention is any non-eukaryotic histonelike protein. By way of non-limiting examples, these include HU protein and IHF (integration host factor). HU and IHF proteins have been identified and cloned from a variety of eubacteria and archaea, including for example Aeromonas proteolytica, Bacillus caldolyticus, Bacillus caldotenax, Bacillus cereus, Bacillus globigii, Bacillus stearothermophilus, Bacillus subtilis, Bifidobacterium longum, Borrelia burgdorferi, Campylobacter jejuni, Escherichia coli, Mycoplasma gallisepticum, Neisseria gonorrhoeae, Pseudomonas aeruginosa, Pseudomonas putida, Rhodobacter capsulatus, Salmonella typhimurium, Serratia marcescens, and Thermotoga maritima.

Histones. Another class of DNA-binding, Arg- and Lys-rich protein that can be used in the complexes and compositions of the present invention is a histone or mixture of a histones. Any histone protein, including without limitation H1, H2A, H2B, H3 and H4, can be used. The use of histone proteins is described in the following references, all of which are incorporated herein by reference in their entireties: Balicki D, Beutler E. 1997. Histone H2A significantly enhances in vitro DNA transfection. Mol. Med. 3:782-787; Balicki et al. 2000. Histone H2A-mediated transient cytokine gene delivery induces efficient antitumor responses in murine neuroblastoma. Proc Natl Acad Sci USA 97:11500-11504; Balicki et al. 2002. Structure and function correlation in histone H2A peptide-mediated gene transfer. Proc Natl Acad Sci USA 99:7467-7471; Demirhan et al. 1998. Histone-mediated transfer and expression of the HIV-1 tat gene in Jurkat cells. J Hum Virol. 1:430-440; and Zaitsev et al. 2002. Histone HI-mediated transfection: role of calcium in the cellular uptake and intracellular fate of H1-DNA complexes. Acta Histochem 104:85-92. See also U.S. Pat. Nos. 6,180,784 and 5,744,335 (both entitled “Process of transfecting a cell with a polynucleotide mixed with an amphipathic compound and a DNA-binding protein”), both to Wolff, et al.; U.S. Pat. No. 6,458,382 (“Nucleic acid transfer complexes”) to Herweijer, et al.; published PCT application WO 96/14424 (“DNA transfer method”) to Hallybone; and published PCT application WO 99/19502, EP 0 967 288 A1, and EP 0 908 521 A1 (all entitled “Transfection System for the transfer of nucleic acids into cells”), all to Chandra, et al.

The human histone-like protein described in U.S. Pat. Nos. 5,851,799, 5,981,221 and 5,908,831 (all entitled “Histone-like protein), all to Bandman, et al., and the protein and peptide sequences described in U.S. Pat. Nos. 5,945,400 and 6,200,956, and Published PCT application WO 96/25508 (all entitled “Nucleic acid-containing composition; preparation and use thereof”), all to Scherman, et al., can also be used to practice the invention. Chemically modified histone proteins, including by way of non-limiting example galactosylated histones (Chen, et al., Hum Gene Ther 5:429-435, 1994), can be used in the invention.

Nucleic Acids. As noted above, the complexes of the present invention may comprise one or more nucleic acids or nucleic acid molecules, which often will comprise one or more genes of interest, that can be delivered to cells, tissues, organs or organisms using the compositions, complexes and methods of the present invention. As used herein, the term “nucleic acids” (which is used herein interchangeably and equivalently with the term “nucleic acid molecules”) refers to nucleic acids (including DNA, RNA, and DNA-RNA hybrid molecules) that are isolated from a natural source; that are prepared in vitro, using techniques such as PCR amplification or chemical synthesis; that are prepared in vivo, e.g., via recombinant DNA technology; or that are prepared or obtained by any appropriate method. Nucleic acids used in accordance with the invention may be of any shape (linear, circular, etc.) or topology (single-stranded, double-stranded, linear, circular, supercoiled, torsional, nicked, etc.). The term “nucleic acids” also includes without limitation nucleic acid derivatives such as peptide nucleic acids (PNAS) and polypeptide-nucleic acid conjugates; nucleic acids having at least one chemically modified sugar residue, backbone, internucleotide linkage, base, nucleotide, nucleoside, or nucleotide analog or derivative; as well as nucleic acids having chemically modified 5′ or 3′ ends; and nucleic acids having two or more of such modifications. Not all linkages in a nucleic acid need to be identical.

Examples of nucleic acids include without limitation oligonucleotides (including but not limited to antisense oligonucleotides, ribozymes and oligonucleotides useful in RNA interference (RNAi)), aptamers, polynucleotides, artificial chromosomes, cloning vectors and constructs, expression vectors and constructs, gene therapy vectors and constructs, rRNA, tRNA, mRNA, mtRNA, and tmRNA, and the like. For reviews of the latter type of nucleic acid, see Muto A, Ushida C, Himeno H. A bacterial RNA that functions as both a tRNA and an mRNA. Trends Biochem Sci. 23:25-29, 1998; and Gillet R, Felden B. Emerging views on tmRNA-mediated protein tagging and ribosome rescue. Mol. Microbiol. 42:879-885, 2001.

Oligonucleotides. As used in the present invention, an oligonucleotide is a synthetic or biologically produced molecule comprising a covalently linked sequence of nucleotides which may be joined by a phosphodiester bond between the 3′ position of the pentose of one nucleotide and the 5′ position of the pentose of the adjacent nucleotide. As used herein, the term “oligonucleotide” includes natural nucleic acid molecules (i.e., DNA and RNA) as well as non-natural or derivative molecules such as peptide nucleic acids, phophothioate-containing nucleic acids, phosphonate-containing nucleic acids and the like. In addition, oligonucleotides of the present invention may contain modified or non-naturally occurring sugar residues (e.g., arabinose) and/or modified base residues. The term oligonucleotide encompasses derivative molecules such as nucleic acid molecules comprising various natural nucleotides, derivative nucleotides, modified nucleotides or combinations thereof. Oligonucleotides of the present invention may also comprise blocking groups which prevent the interaction of the molecule with particular proteins, enzymes or substrates.

Oligonucleotides include without limitation RNA, DNA and hybrid RNA-DNA molecules having sequences that have minimum lengths of e nucleotides, wherein “e” is any whole integer from about 2 to about 15, and maximum lengths of about f nucleotides, wherein “f” is any whole integer from about 2 to about 200. In general, a minimum of about 6 nucleotides, preferably about 10, and more preferably about 12 to about 15 nucleotides, is desirable to effect specific binding to a complementary nucleic acid strand.

In general, oligonucleotides may be single-stranded (ss) or double-stranded (ds) DNA or RNA, or conjugates (e.g., RNA molecules having 5′ and 3′ DNA “clamps”) or hybrids (e.g., RNA:DNA paired molecules), or derivatives (chemically modified forms thereof). Single-stranded DNA is often preferred, as DNA is less susceptible to nuclease degradation than RNA. Similarly, chemical modifications that enhance the specificity or stability of an oligonucleotide are preferred in some applications of the invention.

Certain types of oligonucleotides are of particular utility in the compositions and complexes of the present invention, including but not limited to antisense oligonucleotides, ribozymes, interfering RNAs and aptamers.

Antisense Oligonucleotides. Nucleic acid molecules suitable for use in the present invention include antisense oligonucleotides. In general, antisense oligonucleotides comprise nucleotide sequences sufficient in identity and number to effect specific hybridization with a preselected nucleic acid. Antisense oligonucleotides are generally designed to bind either directly to mRNA transcribed from, or to a selected DNA portion of, a targeted gene, thereby modulating the amount of protein translated from the mRNA or the amount of mRNA transcribed from the gene, respectively. Antisense oligonucleotides may be used as research tools, diagnostic aids, and therapeutic agents.

Antisense oligonucleotides used in accordance with the present invention typically have sequences that are selected to be sufficiently complementary to the target mRNA sequence so that the antisense oligonucleotide forms a stable hybrid with the mRNA and inhibits the translation of the mRNA sequence, preferably under physiological conditions. It is preferred but not necessary that the antisense oligonucleotide be 100% complementary to a portion of the target gene sequence. However, the present invention also encompasses the production and use of antisense oligonucleotides with a different level of complementarity to the target gene sequence, e.g., antisense oligonucleotides that are at least about 50% complementary, at least about 55% complementary, at least about 60% complementary, at least about 65% complementary, at least about 70% complementary, at least about 75% complementary, at least about 80% complementary, at least about 85% complementary, at least about 90% complementary, at least about 91% complementary, at least about 92% complementary, at least about 93% complementary, at least about 94% complementary, at least about 95% complementary, at least about 96% complementary, at least about 97% complementary, at least about 98% complementary, or at least about 99% complementary, to the target gene sequence. In certain embodiments, the antisense oligonucleotide hybridizes to an isolated target mRNA under the following conditions: blots are first incubated in prehybridization solution (5.times.SSC; 25 mM NaPO.sub.4, pH 6.5; 1.times.Denhardt's solution; and 1% SDS) at 42.degree. C. for at least 2 hours, and then hybridized with radiolabelled cDNA probes or oligonucleotide probes (1.times.10.sup.6 cpm/ml of hybridization solution) in hybridization buffer (5.times.SSC; 25 mM NaPO.sub.4, pH 6.5; 1.times.Denhardt's solution; 250 ug/ml total RNA; 50% deionized formamide; 1% SDS; and 10% dextran sulfate). Hybridization for 18 hours at 30-42.degree. C. is followed by washing of the filter in 0.1-6.times.SSC, 0.1% SDS three times at 25-55.degree. C. The hybridization temperatures and stringency of the wash will be determined by the percentage of the GC content of the oligonucleotides in accord with the guidelines described by Sambrook et al. (Molecular Cloning: A Laboratory Manual, 2nd edition, 1989, Cold Spring Harbor Laboratory Press, Plainview, N.Y.).

Representative teachings regarding the synthesis, design, selection and use of antisense oligonucleotides include without limitation U.S. Pat. No. 5,789,573, Antisense Inhibition of ICAM-1, E-Selectin, and CMV IE1/IE2, to Baker et al.; U.S. Pat. No. 6,197,584, Antisense Modulation of CD40 Expression, to Bennett et al.; and Ellington, 1992, Current Protocols in Molecular Biology, 2nd Ed., Ausubel et al., eds., Wiley Interscience, New York, Units 2.11 and 2.12.

Ribozymes. Nucleic acid molecules suitable for use in the present invention also include ribozymes. In general, ribozymes are RNA molecules having enzymatic activities usually associated with cleavage, splicing or ligation of nucleic acid sequences. The typical substrates for ribozymes are RNA molecules, although ribozymes may catalyze reactions in which DNA molecules (or maybe even proteins) serve as substrates. Two distinct regions can be identified in a ribozyme: the binding region which gives the ribozyme its specificity through hybridization to a specific nucleic acid sequence (and possibly also to specific proteins), and a catalytic region which gives the ribozyme the activity of cleavage, ligation or splicing. Ribozymes which are active intracellularly work in cis, catalyzing only a single turnover, and are usually self-modified during the reaction. However, ribozymes can be engineered to act in trans, in a truly catalytic manner, with a turnover greater than one and without being self-modified. Owing to the catalytic nature of the ribozyme, a single ribozyme molecule cleaves many molecules of target RNA and therefore therapeutic activity is achieved in relatively lower concentrations than those required in an antisense treatment (WO 96/23569).

Representative teachings regarding the synthesis, design, selection and use of ribozymes include without limitation U.S. Pat. No. 4,987,071, RNA ribozyme polymerases, dephosphorylases, restriction endoribonucleases and methods, to Cech et al.; and U.S. Pat. No. 5,877,021, B7-1 Targeted Ribozymes, to Stinchcomb et al.; the disclosures of all of which are incorporated herein by reference in their entireties.

Nucleic Acids for RNAi (RNAi Molecules). Nucleic acid molecules suitable for use in the present invention also include nucleic acid molecules, particularly oligonucleotides, useful in RNA interference (RNAi). In general, RNAi is one method for analyzing gene function in a sequence-specific manner. For reviews, see Tuschl, T., Chembiochem. 2:239-245 (2001), and Cullen, B. R., Nat. Immunol. 3:597-599 (2002). RNA-mediated gene-specific silencing has been described in a variety of model organisms, including nematodes (Parrish, S., et al., Mol Cell 6:1077-1087 (2000); Tabara, H., et al., Cell 99:123-132 (1999); in plants, i.e., “co-suppression” (Napoli, C., et al., Plant Cell 2:279-289 (1990)) and post-transcriptional or homologous gene silencing (Hamilton, A. J. and D.C. Baulcombe, Science 286:950-952 (1999); Hamilton, et al., EMBO J. 21:4671-4679 (2002)) (PTGS or HGS, respectively) in plants; and in fungi, i.e., “quelling” (Romano, N. and G. Macino, Mol Microbiol 6:3343-3353 (1992)). Examples of suitable interfering RNAs include siRNAs, shRNAs and stRNAs. As one of ordinary skill will readily appreciate, however, other RNA molecules having analogous interfering effects are also suitable for use in accordance with this aspect of the present invention.

Small Interfering RNA (siRNA). RNAi is mediated by double stranded RNA (dsRNA) molecules that have sequence-specific homology to their “target” mRNAs (Caplen, N. J., et al., Proc Natl Acad Sci USA 98:9742-9747 (2001)). Biochemical studies in Drosophila cell-free lysates indicates that the mediators of RNA-dependent gene silencing are 21-25 nucleotide “small interfering” RNA duplexes (siRNAs). Accordingly, siRNA molecules are advantageously used in the compositions, complexes and methods of the present invention. The siRNAs are derived from the processing of dsRNA by an RNase known as Dicer (Bernstein, E., et al., Nature 409:363-366 (2001)). It appears that siRNA duplex products are recruited into a multi-protein siRNA complex termed RISC(RNA Induced Silencing Complex). Without wishing to be bound by any particular theory, it is believed that a RISC is guided to a target mRNA, where the siRNA duplex interacts sequence-specifically to mediate cleavage in a catalytic fashion (Bernstein, E., et al., Nature 409:363-366 (2001); Boutla, A., et al., Curr Biol 11:1776-1780 (2001); Hammond et al., 2000).

RNAi has been used to analyze gene function and to identify essential genes in mammalian cells (Elbashir, et al., Methods 26:199-213 (2002); Harborth, et al., J Cell Sci 114:4557-4565 (2001)), including by way of non-limiting example neurons (Krichevsky, A. M. and Kosik, K. S., Proc Natl Acad Sci USA 99:11926-11929 (2002)). RNAi is also being evaluated for therapeutic modalities, such as inhibiting or block the infection, replication and/or growth of viruses, including without limitation poliovirus (Gitlin, et al, Nature 418:379-380 (2002)) and HIV (Capodici, et al., J Immunol 169:5196-5201 (2002)), and reducing expression of oncogenes (e.g., the bcr-abl gene; Scherr, et al., Blood September 26 (epub ahead of print) (2002)). RNAi has been used to modulate gene expression in mammalian (mouse) and amphibian (Xenopus) embryos (Calegari, et al., Proc Natl Acad Sci USA 99:14236-14240 (2002), and Zhou, et al., Nucleic Acids Res 30:1664-1669 (2002), respectively), and in postnatal mice (Lewis, et al., Nat Genet. 32:107-108 (2002)), and to reduce trangsene expression in adult transgenic mice (McCaffrey, et al., Nature 418:38-39 (2002)).

Molecules that mediate RNAi, including without limitation siRNA, can be produced in vitro by chemical synthesis (Hohjoh, H., FEBS Lett 521:195-199 (2002)), hydrolysis of dsRNA (Yang, et al., Proc Natl Acad Sci USA 99:9942-9947 (2002)), by in vitro transcription with T7 RNA polymerase (Donze, 0. and Picard, D., Nucleic Acids Res 30:e46. (2002); Yu, et al., Proc Natl Acad Sci USA 99:6047-6052 (2002)), and by hydrolysis of double-stranded RNA using a nuclease such as E. coli RNase III (Yang, et al., Proc Natl Acad Sci USA 99:9942-9947 (2002)). RNAi molecules can also be expressed inside cells by endogenous RNA polymerases, using for example RNA Pol III which acts on the U6 RNA promoter (Yu, et al., Proc Natl Acad Sci USA 99:6047-6052 (2002); Paul, et al., Nat Biotechnol 20:505-508 (2002)). For example, the commercially available GeneSuppressor System (IMGENEX, San Diego, Calif.) uses vectors comprising the U6 promoter to generate RNAi molecules in vivo. Viral vectors for siRNA (Xia, et al., Nat Biotechnol 20:1006-1010 (2002)) including, by way of non-limiting example, retroviruses (Devroe, E. and Silver, P. A., BMC Biotechnol 2:15 (2002)), have also been described. Methods have been described for determining the efficacy and specificity of siRNAs in cell culture and in vivo (Bertrand, et al., Biochem Biophys Res Commun 296:1000-1004 (2002); Lassus, et al., Sci STKE 2002 (147):PL13 (2002); Leirdal, M. and Sioud, M., Biochem Biophys Res Commun 295:744-748 (2002)).

Because the Dicer RNase facilitates siRNA production, it is expected that cells that express Dicer will demonstrate a quicker and/or more robust response to dsRNA-mediated RNAi, and that cells that overexpress Dicer will respond even more quickly and/or more robustly. Overexpression of Dicer may be achieved by cloning a gene for a Dicer protein (e.g., the Drosophila DCR-1 gene), or orthologs or homologs thereof, into an expression vector or cassette that is placed into a cell of choice. Examples of cloned DCR genes include without limitation homologs and orthologs of DCR from mice (Nicholson, R. H. and Nicholson, A. W., Mamm. Genome 13:67-73 (2002)), accession No. NM 148948; humans (Nagase, T., et al., DNA Res. 6:63-70 (1999)), accession No. NM 030621; as well as the Drosophila Dicer-2 (DCR-2) gene (Adams, et al, Science 287:2185-2195 (2000)), accession No. NM 079054.

In another embodiment, therapeutic nucleic acid molecules (e.g., siNA molecules) delivered exogenously optimally are stable within cells until reverse transcription of the RNA has been modulated long enough to reduce the levels of the RNA transcript. The nucleic acid molecules are resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

In yet another embodiment, siNA molecules having chemical modifications that maintain or enhance enzymatic activity of proteins involved in RNAi are used. Such nucleic acids are also generally more resistant to nucleases than unmodified nucleic acids. Thus, in vitro and/or in vivo the activity should not be significantly lowered.

In one embodiment, nucleic acid molecules of the invention that act as mediators of the RNA interference gene silencing response are double-stranded nucleic acid molecules. In another embodiment, the siNA molecules of the invention consist of duplexes containing about 19 base pairs between oligonucleotides comprising about 19 to about 25 (e.g., about 19, 20, 21, 22, 23, 24 or 25) nucleotides. In yet another embodiment, siNA molecules of the invention comprise duplexes with overhanging ends of about 1 to about 3 (e.g., about 1, 2, or 3) nucleotides, for example, about 21-nucleotide duplexes with about 19 base pairs and 3′-terminal mononucleotide, dinucleotide, or trinucleotide overhangs.

In one embodiment, a siNA molecule of the invention comprises modified nucleotides while maintaining the ability to mediate RNAi. The modified nucleotides can be used to improve in vitro or in vivo characteristics such as stability, activity, and/or bioavailability. For example, a siNA molecule of the invention can comprise modified nucleotides as a percentage of the total number of nucleotides present in the siNA molecule. As such, a siNA molecule of the invention can generally comprise about 5% to about 100% modified nucleotides (e.g., 5%, 10%, 15%, 20%, 25%, 30%, 35%, 40%, 45%, 50%, 55%, 60%, 65%, 70%, 75%, 80%, 85%, 90%, 95% or 100% modified nucleotides). The actual percentage of modified nucleotides present in a given siNA molecule will depend on the total number of nucleotides present in the siNA. If the siNA molecule is single stranded, the percent modification can be based upon the total number of nucleotides present in the single stranded siNA molecules. Likewise, if the siNA molecule is double stranded, the percent modification can be based upon the total number of nucleotides present in the sense strand, antisense strand, or both the sense and antisense strands.

Short Hairpin RNAs (shRNAs). Paddison, P. J., et al, Genes & Dev. 16:948-958 (2002) have used small RNA molecules folded into hairpins as a means to effect RNAi. Accordingly, such short hairpin RNA (shRNA) molecules are also advantageously used in the compositions, complexes and methods of the present invention. The length of the stem and loop of functional shRNAs varies; stem lengths can range anywhere from about 25 to about 30 nt, and loop size can range between 4 to about 25 nt without affecting silencing activity. While not wishing to be bound by any particular theory, it is believed that these shRNAs resemble the dsRNA products of the Dicer RNase and, in any even, have the same capacity for inhibiting expression of a specific gene.

In order to express siRNA and shRNA long-term in vivo for, by way of non-limiting example, gene therapy and developmental studies, plasmids that express these RNAs have been generated. Expression vectors that continually express siRNAs in stably transfected mammalian cells have been developed. Other plasmids have been engineered to express small hairpin RNAs (shRNAs) lacking poly (A) tails. Transcription of shRNAs is initiated at a polymerase III (pol II) promoter and is believed to be terminated at position 2 of a 4-5-thymine transcription termination site. Upon expression, shRNAs are thought to fold into a stem-loop structure with 3′ UU-overhangs. Subsequently, the ends of these shRNAs are processed, converting the shRNAs into about 21 nt siRNA-like molecules. The siRNA-like molecules can, in turn, bring about gene-specific silencing in the transfected cells, which may be, by way of non-limiting example, mammalian or human cells.

Small Temporally Regulated RNAs (stRNAs). Another group of small RNAs suitable for use in the compositions, complexes and methods of the present invention are the small temporally regulated RNAs (stRNAs). In general, stRNAs comprise from about 20 to about 30 nt (Banerjee and Slack, Control of development timing by small temporal RNAs: A paradigm for RNA-mediated regulation of gene expression, Bioessays 24:119-129, 2002). Unlike siRNAs, stRNAs down-regulate expression of a target mRNA after the initiation of translation without degrading the mRNA.

Design and Synthesis of siRNA, shRNA, stRNA, Antisense and Other Oligonucleotides. One or more of the following guidelines may be used in designing the sequence of siRNA and other nucleic acids designed to bind to a target mRNA, e.g., shRNA, stRNA, antisense oligonucleotides, ribozymes, and the like, that are advantageously used in accordance with the present invention.

Nucleic acids that mediate RNAi may be synthesized in vitro using methods to produce oligonucleotides and other nucleic acids, as is described elsewhere herein. In addition, dsRNA and other molecules that mediate iRNA are available from commercial vendors, such as Ribopharma AG (Kulmach, Germany), Eurogentec (Seraing, Belgium) and Sequitur (Natick, Mass.). Eurogentec offers siRNA that has been labeled with fluorophores (e.g., HEX/TET; 5′ Fluorescein, 6-FAM; 3′ Fluorescein, 6-FAM; Fluorescein dT internal; 5′ TAMRA, Rhodamine; 3′ TAMRA, Rhodamine), and these examples of fluorescent dsRNA that can be used in the invention.

Aptamers. Traditionally, techniques for detecting and purifying target molecules have used polypeptides, such as antibodies, that specifically bind such targets. Nucleic acids have long been known to specifically bind other nucleic acids (e.g., ones having complementary sequences). However, nucleic acids that bind non-nucleic target molecules have been described and are generally referred to as aptamers. See, e.g., Blackwell, T. K., et al., Science (1990) 250:1104-1110; Blackwell, T. K., et al., Science (1990) 250:1149-1152; Tuerk, C., and Gold, L., Science (1990) 249:505-510; Joyce, G. F., Gene (1989) 82:83-87. Accordingly, nucleic acid molecules (e.g., oligonucleotides) suitable for use in the present invention also include aptamers. As applied to aptamers, the term “binding” specifically excludes the “Watson-Crick”-type binding interactions (i.e., A:T and G:C base-pairing) traditionally associated with the DNA double helix.

The term “aptamer” thus refers to a nucleic acid or a nucleic acid derivative that specifically binds to a target molecule, wherein the target molecule is either (i) not a nucleic acid, or (ii) a nucleic acid or structural element thereof that is bound by the aptamer through mechanisms other than duplex- or triplex-type base pairing.

In general, techniques for identifying aptamers involve incubating a preselected non-nucleic acid target molecule with mixtures (2 to 50 members), pools (50 to 5,000 members) or libraries (50 or more members) of different nucleic acids that are potential aptamers under conditions that allow complexes of target molecules and aptamers to form. By “different nucleic acids” it is meant that the nucleotide sequence of each potential aptamer may be different from that of any other member, that is, the sequences of the potential aptamers are random with respect to each other. Randomness can be introduced in a variety of manners such as, e.g., mutagenesis, which can be carried out in vivo by exposing cells harboring a nucleic acid with mutagenic agents, in vitro by chemical treatment of a nucleic acid, or in vitro by biochemical replication (e.g., PCR) that is deliberately allowed to proceed under conditions that reduce fidelity of replication process; randomized chemical synthesis, i.e., by synthesizing a plurality of nucleic acids having a preselected sequence that, with regards to at least one position in the sequence, is random. By “random at a position in a preselected sequence” it is meant that a position in a sequence that is normally synthesized as, e.g., as close to 100% A as possible (e.g., 5′-C-T-T-A-G-T-3′) (SEQ ID NO:1), is allowed to be randomly synthesized at that position (C-T-T-N-G-T, wherein N indicates a randomized position) (SEQ ID NO:2). At a randomized position, for example, the synthesizing reaction contains 25% each of A, T, C and G; or x % A, w % T, y % C and z % G, wherein x+w+y+z=100. The randomization at the position may be complete (i.e., x=y=w=z=25%) or stoichastic (i.e., at least one of x, w, y and z is not 25%).

In later stages of the process, the sequences are increasingly less randomized and consensus sequences may appear; in any event, it is preferred to ultimately obtain an aptamer having a unique nucleotide sequence.

Aptamers and pools of aptamers are prepared, identified, characterized and/or purified by any appropriate technique, including those utilizing in vitro synthesis, recombinant DNA techniques, PCR amplification, and the like. After their formation, target:aptamer complexes are then separated from the uncomplexed members of the nucleic acid mixture, and the nucleic acids that can be prepared from the complexes are candidate aptamers (at early stages of the technique, the aptamers generally being a population of a multiplicity of nucleotide sequences having varying degrees of specificity for the target). The resulting aptamer (mixture or pool) is then substituted for the starting aptamer (library or pool) in repeated iterations of this series of steps. When a limited number (e.g., a pool or mixture, preferably a mixture with less than 10 members, most preferably 1) of nucleic acids having satisfactory specificity is obtained, the aptamer is sequenced and characterized. Pure preparations of a given aptamer are generated by any appropriate technique (e.g., PCR amplification, in vitro chemical synthesis, and the like).

For example, Tuerk and Gold (Science (1990) 249:505-510) describe the use of a procedure termed “systematic evolution of ligands by exponential enrichment” (SELEX). In this method, pools of nucleic acid molecules that are randomized at specific positions are subjected to selection for binding to a nucleic acid-binding protein (see, e.g., PCT International Publication No. WO 91/19813 and U.S. Pat. No. 5,270,163). The oligonucleotides so obtained are sequenced and otherwise characterization. Kinzler, K. W., et al. (Nucleic Acids Res. (1989) 17:3645-3653) used a similar technique to identify synthetic double-stranded DNA molecules that are specifically bound by DNA-binding polypeptides. Ellington, A. D., et al. (Nature (1990) 346: 818-822) describe the production of a large number of random sequence RNA molecules and the selection and identification of those that bind specifically to specific dyes such as Cibacron blue.

Another technique for identifying nucleic acids that bind non-nucleic target molecules is the oligonucleotide combinatorial technique described by Ecker, D. J. et al. (Nuc. Acids Res. 21, 1853 (1993)) known as “synthetic unrandomization of randomized fragments” (SURF), which is based on repetitive synthesis and screening of increasingly simplified sets of oligonucleotide analogue libraries, pools and mixtures (Tuerk, C. and Gold, L. (Science 249, 505 (1990)). The starting library consists of oligonucleotide analogues of defined length with one position in each pool containing a known analogue and the remaining positions containing equimolar mixtures of all other analogues. With each round of synthesis and selection, the identity of at least one position of the oligomer is determined until the sequences of optimized nucleic acid ligand aptamers are discovered.

Once a particular candidate aptamer has been identified through a SURF, SELEX or any other technique, its nucleotide sequence can be determined (as is known in the art), and its three-dimensional molecular structure can be examined by nuclear magnetic resonance (NMR). These techniques are explained in relation to the determination of the three-dimensional structure of a nucleic acid ligand that binds thrombin in Padmanabhan, K. et al., J. Biol. Chem. 24, 17651 (1993); Wang, K. Y. et al., Biochemistry 32, 1899 (1993); and Macaya, R. F. et al., Proc. Nat'l. Acad. Sci. USA 90, 3745 (1993). Selected aptamers may be resynthesized using one or more modified bases, sugars or backbone linkages. Aptamers consist essentially of the minimum sequence of nucleic acid needed to confer binding specificity, but may be extended on the 5′ end, the 3′ end, or both, or may be otherwise derivatized or conjugated.

Oligonucleotide Synthesis. The oligonucleotides used in accordance with the present invention can be conveniently and routinely made through the well-known technique of solid-phase synthesis. Equipment for such synthesis is sold by several vendors including, for example, Applied Biosystems (Foster City, Calif.). Other methods for such synthesis that are known in the art may additionally or alternatively be employed. It is well known to use similar techniques to prepare oligonucleotides such as the phosphorothioates and alkylated derivatives. By way of non-limiting example, see, e.g., U.S. Pat. No. 4,517,338 (Multiple reactor system and method for polynucleotide synthesis) to Urdea et al., and U.S. Pat. No. 4,458,066 (Process for preparing polynucleotides) to Caruthers et al.; Lyer R P, Roland A, Zhou W, Ghosh K. Modified oligonucleotides—synthesis-, properties and applications. Curr Opin Mol. Ther. 1:344-358, 1999; Verma S, Eckstein F. Modified oligonucleotides: synthesis and strategy for users. Annu Rev Biochem. 67:99-134, 1998; Pfleiderer W, Matysiak S, Bergmann F, Schnell R. Recent progress in oligonucleotide synthesis. Acta Biochim Pol. 43:37-44, 1996; Warren W J, Vella G. Principles and methods for the analysis and purification of synthetic deoxyribonucleotides by high-performance liquid chromatography. Mol. Biotechnol. 4:179-199, 1995; Sproat B S. Chemistry and applications of oligonucleotide analogues. J. Biotechnol. 41:221-238, 1995; De Mesmaeker A, Altmann K H, Waldner A, Wendeborn S. Backbone modifications in oligonucleotides and peptide nucleic acid systems. Curr Opin Struct Biol. 5:343-355, 1995; Charubala R, Pfleiderer W. Chemical synthesis of 2′,5′-oligoadenylate analogues. Prog Mol Subcell Biol. 14:114-138, 1994; Sonveaux E. Protecting groups in oligonucleotide synthesis. Methods Mol. Biol. 26:1-71, 1994; Goodchild J. Conjugates of oligonucleotides and modified oligonucleotides: a review of their synthesis and properties. Bioconjug Chem. 1:165-187, 1990; Thuong N T, Asseline U. Chemical synthesis of natural and modified oligodeoxynucleotides. Biochimie. 67:673-684, 1985; Itakura K, Rossi J J, Wallace R B. Synthesis and use of synthetic oligonucleotides. Annu Rev Biochem. 53:323-356, 1984; Caruthers M H, Beaucage S L, Becker C, Efcavitch J W, Fisher E F, Galluppi G, Goldman R, deHaseth P, Matteucci M, McBride L, et al. Deoxyoligonucleotide synthesis via the phosphoramidite method. Gene Amplif Anal. 3:1-26, 1983; Ohtsuka E, Ikehara M, Soll D. Recent developments in the chemical synthesis of polynucleotides. Nucleic Acids Res. 10:6553-6560, 1982; and Kossel H. Recent advances in polynucleotide synthesis. Fortschr Chem Org. Naturst. 32:297-508, 1975.

Oligonucleotides and other nucleic acids having accessory elements can also be prepared for advantageous use in the compositions, complexes and methods of the present invention. Some such accessory elements can specifically bind or otherwise interact with another molecule for a variety of purposes, including without limitation:

Intracellular transport. For example, a nucleotide sequence that localizes nucleic acids to mitochondria is described in U.S. Pat. No. 5,569,754;

Cellular targeting. For example, the sequence of an aptamer that binds to a cell surface molecule (e.g., a receptor, cellular adhesion protein, membrane lipid, etc.) can be included in order to direct the oligonucleotide complex to a particular type of cell;

Delivery of DNA-binding proteins. For example, a nucleotide sequence that specifically binds a transcription factor can be included in order to effect the delivery of the transcription factor at the same time as the other components of the complex;

Delivery of recombination proteins. As an example, a site that specifically binds a recombination protein can be included. The recombination protein can be a recombinase per se (e.g., lambda integrase and related site-specific recombinases) or a protein that facilitates or enhances recombination (e.g., a histonelike protein, such as Integration Host Factor, IHF). In one embodiment, a histonelike protein (e.g., IHF) and a site-specific recombinase (e.g., lambda integrase or Xis) are incorporated into one or more complexes, and cells are transfected therewith. The presence of IHF in transfected cells increases the amount of site-specific recombination mediated by the integrase, thereby promoting recombination between specific sites (e.g., attB, attP, attL, attR, etc.) on nucleic acids within the cells (Christ et al., 2002. Site-specific recombination in eukaryotic cells mediated by mutant lambda integrases: implications for synaptic complex formation and the reactivity of episomal DNA segments. J Mol Biol 319:305-314). Such cells include, without limitation, embryonic cells, such as stem cells (Christ N, Droge P. 2002. Genetic manipulation of mouse embryonic stem cells by mutant lambda integrase. Genesis 32:203-208). In another embodiment, mutants of lambda integrase that have activity in the absence of IHF are used (Lorbach et al, 2000. Site-specific recombination in human cells catalyzed by phage lambda integrase mutants. J Mol Biol 296:1175-81).

Chemical Modifications of Nucleic Acids. In certain embodiments, oligonucleotides used in accordance with the present invention may comprise one or more chemical modifications including with neither limitation nor exclusivity base modifications, sugar modifications, and backbone modifications. In addition, a variety of molecules can be conjugated to the oligonucleotides; see, e.g., the descriptions of chemical conjunction of fluorophores to oligonucleotides that are present throughout the present disclosure. Other suitable modifications include but are not limited to base modifications, sugar modifications, backbone modifications, and the like.

Base Modifications. In certain embodiments, the oligonucleotides used in the present invention can comprise one or more base modifications. For example, the base residues in aptamers may be other than naturally occurring bases (e.g., A, G, C, T, U, and the like). Derivatives of purines and pyrimidines are known in the art; an exemplary but not exhaustive list includes aziridinylcytosine, 4-acetylcytosine, 5-fluorouracil, 5-bromouracil, 5-carboxymethylaminomethyl-2-thiouracil, 5-carboxymethylaminomethyluracil, inosine (and derivatives thereof), N6-isopentenyladenine, 1-methyladenine, 1-methylpseudouracil, 1-methylguanine, 1-methylinosine, 2,2-dimethylguanine, 2-methyladenine, 2-methylguanine, 7-methylguanine, 3-methylcytosine, 5-methylcytosine (5MC), N6-methyladenine, 5-methylaminomethyluracil, 5-methoxyaminomethyl-2-thiouracil, beta-D-mannosylqueosine, 5-methoxyuracil, 2-methylthio-N-6-isopentenyladenine, uracil-5-oxyacetic acid methylester, pseudouracil, queosine, 2-thiocytosine, 5-methyl-2-thiouracil, 2-thiouracil, 4-thiouracil, 5-methyluracil, uracil-5-oxyacetic acid, and 2,6-diaminopurine. In addition to nucleic acids that incorporate one or more of such base derivatives, nucleic acids having nucleotide residues that are devoid of a purine or a pyrimidine base may also be included in oligonucleotides and other nucleic acids.

Sugar Modifications. The oligonucleotides used in the present invention can also (or alternatively) comprise one or more sugar modifications. For example, the sugar residues in oligonucleotides and other nucleic acids may be other than conventional ribose and deoxyribose residues. By way of non-limiting example, substitution at the 2′-position of the furanose residue enhances nuclease stability. An exemplary, but not exhaustive list, of modified sugar residues includes 2′ substituted sugars such as 2′-O-methyl-, 2′-O-alkyl, 2′-O-allyl, 2′-S-alkyl, 2′-S-allyl, 2′-fluoro-, 2′-halo, or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs and a basic nucleoside analogs such as methyl riboside, ethyl riboside or propylriboside.

Backbone Modifications. The oligonucleotides used in the invention can also (or alternatively) comprise one or more backbone modifications. Chemically modified backbones of oligonucleotides and other nucleic acids include, by way of non-limiting example, phosphorothioates, chiral phosphorothioates, phosphorodithioates, phosphotriesters, aminoalkylphos-photriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotri-esters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Chemically modified backbones that do not contain a phosphorus atom have backbones formed by short chain alkyl or cycloalkyl internucleoside linkages, mixed heteroatom and alkyl or cycloalkyl internucleoside linkages, or one or more short chain heteroatomic or heterocyclic internucleoside linkages, including without limitation morpholino linkages; siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; and amide backbones.

Vectors and Constructs. In certain embodiments, the nucleic acid molecules of the invention are provided as vectors, particularly cloning vectors, expression vectors or gene therapy vectors. Vectors according to this aspect of the invention can be double-stranded or single-stranded and which may be DNA, RNA, or DNA/RNA hybrid molecules, in any conformation including but not limited to linear, circular, coiled, supercoiled, torsional, nicked and the like. These vectors of the invention include but are not limited to plasmid vectors and viral vectors, such as a bacteriophage, baculovirus, retrovirus, lentivirus, adenovirus, vaccinia virus, semliki forest virus and adeno-associated virus vectors, all of which are well-known and can be purchased from commercial sources (Invitrogen; Carlsbad, Calif.; Promega, Madison Wis.; Stratagene, La Jolla Calif.).

In accordance with the invention, any vector may be used to construct the cloning vectors and expression vectors of the invention. In particular, vectors known in the art and those commercially available (and variants or derivatives thereof) may in accordance with the invention be engineered to include one or more recombination sites for use in the methods of the invention. Such vectors may be obtained from, for example, Vector Laboratories Inc., Invitrogen, Promega, Novagen, NEB, Clontech, Boehringer Mannheim, Pharmacia, EpiCenter, OriGenes Technologies Inc., Stratagene, Perkin Elmer, Pharmingen, Research Genetics. General classes of vectors of particular interest include prokaryotic and/or eukaryotic cloning vectors, expression vectors, fusion vectors, two-hybrid or reverse two-hybrid vectors, shuttle vectors for use in different hosts, mutagenesis vectors, transcription vectors, vectors for receiving large inserts and the like. Other vectors of interest include viral origin vectors (M13 vectors, bacterial phage .lamda. vectors, adenovirus vectors, and retrovirus vectors), high, low and adjustable copy number vectors, vectors which have compatible replicons for use in combination in a single host (pACYC 184 and pBR322) and eukaryotic episomal replication vectors (pCDM8).

Particular vectors of interest include prokaryotic expression vectors such as pProEx-HT, pcDNA II, pSL301, pSE280, pSE380, pSE420, pTrcHisA, B, and C, pRSET A, B, and C (Invitrogen Corporation), pGEMEX-1, and pGEMEX-2 (Promega, Inc.), the pET vectors (Novagen, Inc.), pTrc99A, pKK223-3, the pGEX vectors, pEZZ18, pRIT2T, and pMC1871 (Pharmacia, Inc.), pKK233-2 and pKK388-1 (Clontech, Inc.), and variants and derivatives thereof. Vectors can also be made from eukaryotic expression vectors such as pYES2, pAC360, pBlueBacHis A, B, and C, pVL1392, pBsueBacIII, pCDM8, pcDNA1, pZeoSV, pcDNA3 pREP4, pCEP4, pEBVHis, pFastBac, pFastBac HT, pFastBac DUAL, pSFV, and pTet-Splice (Invitrogen), pEUK-C1, pPUR, pMAM, pMAMneo, pBI101, pBI121, pDR2, pCMVEBNA, and pYACneo (Clontech), pSVK3, pSVL, pMSG, pCH110, and pKK232-8 (Pharmacia, Inc.), p3′SS, pXT1, pSG5, pPbac, pMbac, pMC1neo, and pOG44 (Stratagene, Inc.), and variants or derivatives thereof.

Other vectors of particular interest include pUC18, pUC19, pBlueScript, pSPORT, cosmids, phagemids, YACs (yeast artificial chromosomes), BACs (bacterial artificial chromosomes), MACs (mammalian artificial chromosomes), HACs (human artificial chromosomes), P1 (E. coli phage), pQE70, pQE60, pQE9 (Qiagen), pBS vectors, PhageScript vectors, BlueScript vectors, pNH8A, pNH16A, pNH18A, pNH46A (Stratagene), pcDNA3, pSPORT1, pSPORT2, pCMVSPORT2.0 and pSV-SPORT1 (Invitrogen), pGEX, pTrsfus, pTrc99A, pET-5, pET-9, pKK223-3, pKK233-3, pDR540, pRIT5 (Pharmacia), and variants or derivatives thereof.

Additional vectors of interest include pTrxFus, pThioHis, pLEX, pTrcHis, pTrcHis2, pRSET, pBlueBacHis2, pcDNA3.1/H is, pcDNA3.1(−)/Myc-His, pSecTag, pEBVHis, pPIC9K, pPIC3.5K, pAO815, pPICZ, pPICZa, pGAPZ, pGAPZa, pBlueBac4.5, pBlueBacHis2, pMelBac, pSinRep5, pSinHis, pIND, pIND(SP1), pVgRXR, pcDNA2.1. pYES2, pZErO1.1, pZErO-2.1, pCR-Blunt, pSE280, pSE380, pSE420, pVL1392, pVL1393, pCDM8, pcDNA1.1, pcDNA1.1/Amp, pcDNA3.1, pcDNA3.1/Zeo, pSe, SV2, pRc/CMV2, pRc/RSV, pREP4, pREP7, pREP8, pREP9, pREP10, pCEP4, pEBVHis, pCR3.1, pCR2.1, pCR3.1-Uni, and pCRBac from Invitrogen; .lamda.ExCell, .lamda.gt11, pTrc99A, pKK223-3, pGEX-1.lamda.T, pGEX-2T, pGEX-2TK, pGEX-4T-1, pGEX-4T-2, pGEX-4T-3, pGEX-3×, pGEX-5X-1, pGEX-5X-2, pGEX-5X-3, pEZZ18, pRIT2T, pMC1871, pSVK3, pSVL, pMSG, pCH110, pKK232-8, pSL1180, pNEO, and pUC4K from Pharmacia; pSCREEN-1b(+), pT7Blue(R), pT7Blue-2, pCITE-4-abc(+), pOCUS-2, pTAg, pET-32 LIC, pET-30 LIC, pBAC-2 cp LIC, pBACgus-2 cp LIC, pT7Blue-2 LIC, pT7Blue-2, ASCREEN-1, .lamda.BlueSTAR, pET-3abcd, pET-7abc, pET9abcd, pET11abcd, pET12abc, pET-14b, pET-15b, pET-16b, pET-17b-pET-17xb, pET-19b, pET-20b(+), pET-21abcd(+), pET-22b(+), pET-23abcd(+), pET-24abcd(+), pET-25b(+), pET-26b(+), pET-27b(+), pET-28abc(+), pET-29abc(+), pET-30abc(+), pET-31b(+), pET-32abc(+), pET-33b(+), pBAC-1, pBACgus-1, pBAC4x-1, pBACgus4x-1, pBAC-3 cp, pBACgus-2 cp, pBACsurf-1, plg, Signal plg, pYX, Selecta Vecta-Neo, Selecta Vecta-Hyg, and Selecta Vecta-Gpt from Novagen; pLexA, pB42AD, pGBT9, pAS2-1, pGAD424, pACT2, pGAD GL, pGAD GH, pGAD10, pGilda, pEZM3, pEGFP, pEGFP-1, pEGFP-N, pEGFP-C, pEBFP, pGFPuv, pGFP, p6xHis-GFP, pSEAP2-Basic, pSEAP2-Contral, pSEAP2-Promoter, pSEAP2-Enhancer, p.beta.gal-Basic, p.beta.gal-Control, p.beta.gal-Promoter, p.beta.gal-Enhancer, pCMV.beta., pTet-Off, pTet-On, pTK-Hyg, pRetro-Off, pRetro-On, pIRES1neo, pIRES1hyg, pLXSN, pLNCX, pLAPSN, pMAMneo, pMAMneo-CAT, pMAMneo-LUC, pPUR, pSV2neo, pYEX 4T-1/2/3, pYEX-S1, pBacPAK-His, pBacPAK8/9, pAcUW31, BacPAK6, pTrip1Ex, .lamda.gt10, .lamda.gt11, pWE15, and .lamda.Trip1Ex from Clontech; Lambda ZAP II, pBK-CMV, pBK-RSV, pBluescript II KS .+−., pBluescript II SK .+−., pAD-GAL4, pBD-GAL4 Cam, pSurfscript, Lambda FIX II, Lambda DASH, Lambda EMBL3, Lambda EMBL4, SuperCos, pCR-Scrigt Amp, pCR-Script Cam, pCR-Script Direct, pBS .+−., pBC KS .+−., pBC SK .+−., Phagescript, pCAL-n-EK, pCAL-n, pCAL-c, pCAL-kc, pET-3abcd, pET-11 abcd, pSPUTK, pESP-1, pCMVLacI, pOPRSVI/MCS, pOPI3 CAT, pXT1, pSG5, pPbac, pMbac, pMC1neo, pMC1neo Poly A, p0044, pOG45, PFRT.beta.GAL, pNEO.beta.GAL, pRS403, pRS404, pRS405, pRS406, pRS413, pRS414, pRS415, and pRS416 from Stratagene.

Two-hybrid and reverse two-hybrid vectors of particular interest include pPC86, pDBLeu, pDBTrp, pPC97, p2.5, pGAD1-3, pGAD10, pACt, pACT2, pGADGL, pGADGH, pAS2-1, pGAD424, pGBT8, pGBT9, pGAD-GAL4, pLexA, pBD-GAL4, pHISi, pHISi-1, placZi, pB42AD, pDG202, pJK202, pJG4-5, pNLexA, pYESTrp and variants or derivatives thereof. Other suitable vectors will be readily apparent to the skilled artisan.

Cloning Vectors. Cloning vectors according to the invention include plasmids, cosmids, viral or phage DNA molecules or other DNA molecules that are capable of autonomous replication in a host cell, via splicing of vector-borne nucleic acid into the genetic material (chromosomal or extrachromosomal) of the host cell without loss of an essential biological function of the vector, thereby facilitating the replication and cloning of the vector. The cloning vector may further contain a marker suitable for use in the identification of cells transformed with the cloning vector. Markers may be, for example, antibiotic resistance genes, e.g., tetracycline resistance or ampicillin resistance. Clearly, methods of inserting a desired nucleic acid fragment which do not require the use of homologous recombination, transpositions or restriction enzymes (such as, but not limited to, UDG cloning of PCR fragments (U.S. Pat. No. 5,334,575, entirely incorporated herein by reference), T:A cloning, and the like) can also be applied to clone a fragment into a cloning vector to be used according to the present invention. The cloning vector can further contain one or more selectable markers suitable for use in the identification of cells transformed with the cloning vector.

Expression Vectors. Expression vectors according to the invention include vectors that are capable of enhancing the expression of one or more genes that have been inserted or cloned into the vector, upon transformation of the vector into a host. The cloned gene is usually placed under the control of (i.e., operably linked to) certain transcriptional regulatory sequences such as promoter sequences. In certain preferred embodiments in this regard, the vectors provide for specific expression, which may be inducible and/or cell type-specific. Particularly preferred among such vectors are those inducible by environmental factors that are easy to manipulate, such as temperature and nutrient additives. Expression vectors useful in the present invention include chromosomal-, episomal- and virus-derived vectors, e.g., vectors derived from bacterial plasmids or bacteriophages, and vectors derived from combinations thereof, such as cosmids and phagemids.

To produce expression vectors according to this aspect of the invention, one or more gene-containing nucleic acid molecules or oligonucleotide inserts should be operatively linked to an appropriate promoter in the vector (which may be provided by the vector itself (i.e., a “homologous promoter”) or may be exogenous to the vector (i.e., a “heterologous promoter), such as the phage lambda P.sub.L promoter, the E. coli lac, trp and tac promoters, and the like. Other suitable promoters will be known to the skilled artisan. The gene fusion constructs will further contain sites for transcription initiation, termination and, in the transcribed region, a ribosome binding site for translation. The coding portion of the mature transcripts expressed by the constructs will preferably include a translation initiation codon at the beginning, and a termination codon (UAA, UGA or UAG) appropriately positioned at the end, of the polynucleotide to be translated. The expression vectors also preferably include at least one selectable marker. Such markers include tetracycline or ampicillin resistance genes for culturing in E. coli and other bacteria.

Vectors, Compositions and Methods for Gene Therapy. In additional embodiments, the invention provides compositions comprising one or more genetic constructs, including vectors (such as the expression or cloning vectors described above), or one or more of the complexes of the invention, that may be useful in delivering nucleic acid molecules to cells, tissues, organs and organisms for therapeutic or prophylactic purposes. The invention further provides methods for preparing nucleic acid molecules having regions of viral nucleic acids, as well as nucleic acid molecules prepared by such methods and compositions comprising these nucleic acid molecules, useful for the nucleic acid delivery and therapeutic/prophylactic purposes described above and in more detail below.

In one embodiment, the present invention provides methods for treating or preventing a physical disorder in an animal that is suffering from or predisposed to the physical disorder, comprising introducing into the animal one or more of the nucleic acid molecules, complexes or compositions of the invention. According to the invention, an animal, particularly a mammal (preferably a human) that is suffering from, or that is predisposed or susceptible to, a physical disorder may be treated by administering to the animal an effective dose of one or more of the nucleic acid molecules, complexes or compositions of the invention, optionally in combination with a pharmaceutically acceptable carrier or excipient therefor. As used herein, an animal that is “suffering from” a particular physical disorder is defined as an animal that exhibits one or more overt physical symptoms of the disorder that are typically used in the diagnosis or identification of the disorder according to established medical and veterinary procedures and protocols that will be familiar to the ordinarily skilled artisan. Analogously, as used herein, an animal that is “predisposed to” or “susceptible to” a physical disorder is defined as an animal that does not exhibit a plurality of overt physical symptoms of the disorder but that is genetically, physiologically or otherwise at risk for developing the disorder under appropriate physiological and environmental conditions. Hence, whether or not a particular animal is “suffering from,” “predisposed to” or “susceptible to” a particular physical disorder will be apparent to the ordinarily skilled artisan upon determination of the medical history of the animal using methods that are routine in the medical and veterinary arts.

Physical disorders treatable or preventable with the compositions and methods of the present invention include any physical disorder that may be delayed, prevented, cured or otherwise treated by modulating immune system function, particularly activation and/or apoptosis in antigen-presenting cells, in an animal suffering from, or predisposed or susceptible to, the physical disorder. Such physical disorders that may be treatable or preventable using the compositions, complexes and methods of the present invention include, but are not limited to, infectious diseases (particularly bacterial diseases (including without limitation meningitis, pneumonia, tetanus, cholera, typhoid fever, staphylococcal skin infections, streptococcal pharyngitis, scarlet fever, pertussis, diphtheria, tuberculosis, leprosy, rickettsial diseases, bacteremia, bacterial venereal diseases and the like), viral diseases (including without limitation meningitis, AIDS, influenza, rhinitis, hepatitis, polio, pneumonia, yellow fever, Lassa fever, Ebola fever and the like), and/or fungal diseases (including without limitation cryptococcosis, blastomycosis, mucormycosis, histoplasmosis, aspergillosis, and the like), parasitic diseases (including without limitation malaria, Leishmaniasis, filariasis, trypanasomiasis, schistosomiasis, and the like), cancers (such as carcinomas, melanomas, sarcomas, leukemias and the like), and other disorders treatable or preventable using the methods and compositions of the present invention. Analogously, physical disorders that may be treatable or preventable using the present compositions and methods include, but are not limited to, immune system disorders (such as rheumatoid arthritis, multiple sclerosis, systemic lupus erythematosis, Crohn's Disease), and other disorders of analogous etiology. The compositions and methods of the present invention may also be used in the prevention of disease progression, such as in chemoprevention of the progression of a premalignant lesion to a malignant lesion, and to treat an animal suffering from, or predisposed to, other physical disorders that respond to treatment with compositions that activate, or inhibit/delay/prevent or induce apoptosis in, antigen-presenting cells.

In a first such aspect of the invention, the animal suffering from or predisposed to a physical disorder may be treated by introducing into the animal one or more of the nucleic acid molecules of the invention, optionally in the form of a vector and further optionally in the form of a polypeptide-nucleic acid complex of the invention (or a composition of the invention comprising one or more such complexes). This approach, known generically as “gene therapy,” is designed to increase the level of expression of a given gene, generally contained on the nucleic acid molecule and/or in the administered complex, in the cells and/or tissues of the animal, thereby inhibiting, delaying or preventing the progression and/or development of the physical disorder, or to induce the reversal, amelioration or remission of one or more overt symptoms or processes of the physical disorder. Analogous gene therapy approaches have proven effective or to have promise in the treatment of a variety of mammalian diseases such as cystic fibrosis (Drumm, M. L. et al., Cell 62:1227-1233 (1990); Gregory, R. J. et al., Nature 347:358-363 (1990); Rich, D. P. et al., Nature 347:358-363 (1990)), Gaucher disease (Sorge, J. et al., Proc. Natl. Acad. Sci. USA 84:906-909 (1987); Fink, J. K. et al., Proc. Natl. Acad. Sci. USA 87:2334-2338 (1990)), certain forms of hemophilia (Bontempo, F. A. et al., Blood 69:1721-1724 (1987); Palmer, T. D. of al., Blood 73:438-445 (1989); Axelrod, J. H. et al., Proc. Natl. Acad. Sci. USA 87:5173-5177 (1990); Armentano, D. et al., Proc. Natl. Acad. Sci. USA 87:6141-6145 (1990)) and muscular dystrophy (Partridge, T. A. et al., Nature 337:176-179 (1989); Law, P. K. et al., Lancet 336:114-115 (1990); Morgan, J. E. et al., J. Cell Biol. 111:2437-2449 (1990)), and certain cancers such as metastatic melanoma (Rosenberg, S. A. et al., Science 233:1318-1321 (1986); Rosenberg, S. A. et al., N. Eng. J. Med. 319:1676-1680 (1988); Rosenberg, S. A. et al., N. Eng. J. Med. 323:570-578 (1990)).

In carrying out such gene therapy methods of the invention, a variety of vectors, particularly viral vectors, are useful in forming the complexes and compositions of the invention. For example, adenoviruses are especially attractive vehicles for delivering genes to or via respiratory epithelia and the use of such vectors are included within the scope of the invention. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993), present a review of adenovirus-based gene therapy. Bout et al., Human Gene Therapy 5:3-10 (1994), demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication Nos. WO94/12649 and WO 96/17053; U.S. Pat. No. 5,998,205; and Wang et al., Gene Therapy 2:775-783 (1995), the disclosures of all of which are incorporated herein by reference in their entireties. Adeno-associated viruses (AAV) and Herpes viruses, as well as vectors prepared from these viruses have also been proposed for use in gene therapy (Walsh et al., 1993, Proc. Soc. Exp. Biol. Med. 204:289-300; U.S. Pat. No. 5,436,146; Wagstaff et al., Gene Ther. 5:1566-70 (1998)). Herpes viral vectors are particularly useful for applications where gene expression is desired in nerve cells.

In a preferred such approach, one or more nucleic acid and/or one or more polypeptide of the present invention within the polymer complexes of the invention, is introduced into or administered to the animal that is suffering from or predisposed to the physical disorder. Such nucleic acid molecules may be incorporated into a vector or virion suitable for introducing the nucleic acid molecules into the cells or tissues of the animal to be treated, to form a transfection vector. Suitable vectors or virions for this purpose include those derived from retroviruses, adenoviruses, alphaviruses, herpes viruses and adeno-associated viruses. As one of ordinary skill will readily recognize, the complexes of the invention also optionally may be combined with one or more pharmaceutically acceptable excipients or diluents to form a pharmaceutical composition suitable for use in these methods of the invention.

In addition, general methods for construction of gene therapy vectors and the introduction thereof into affected animals for therapeutic purposes may be obtained in the above-referenced publications, the disclosures of which are specifically incorporated herein by reference in their entirety. In one such general method, vectors comprising the nucleic acid molecules of the present invention are directly introduced into the cells or tissues of the affected animal, preferably by injection, inhalation, ingestion or introduction into a mucous membrane via solution; such an approach is generally referred to as “in vivo” gene therapy. Alternatively, cells, tissues or organs, particularly those containing one or more defective or nonfunctioning genes, containing pathological agents (e.g., bacteria, viruses, parasites, yeasts, etc.), or containing cancer cells or tumors, may be removed from the affected animal and placed into culture according to methods that are well-known to one of ordinary skill in the art. The vectors comprising the nucleic acid molecules of the invention, typically comprising one or more therapeutic genes or nucleic acid sequences, may then be introduced into these cells or tissues by any of the methods described generally above for introducing oligonucleotides into a cell or tissue, and, after a sufficient amount of time to allow incorporation of the oligonucleotides, the cells or tissues may then be re-inserted into the affected animal. Since the introduction of the therapeutic genese or nucleic acid sequences is performed outside of the body of the affected animal, this approach is generally referred to as “ex vivo” gene therapy.

For both in vivo and ex vivo gene therapy, the nucleic acid molecules (e.g., oligonucleotides) of the invention may alternatively be operatively linked to a regulatory DNA sequence, which may be a promoter or an enhancer, or a heterologous regulatory DNA sequence such as a promoter or enhancer derived from a gene, cell or organism different from that used as the source of the nucleic acid molecule being used in gene therapy, to form a genetic construct as described above. This genetic construct may then be inserted into a vector, which is then directly introduced into the affected animal in an in vivo gene therapy approach, or into the cells or tissues of the affected animal in an ex vivo approach. In another embodiment, the genetic construct of the invention may be introduced into the cells or tissues of the animal, either in vivo or ex vivo, in a molecular conjugate with a virus (e.g., an adenovirus or an adeno-associated virus) or viral components (e.g., viral capsid proteins; see WO 93/07283). In yet another embodiment, the genetic construct of the invention may be introduced into the animal in the form of a polypeptide-nucleic acid complex of the invention. Alternatively, transfected host cells, which may be homologous or heterologous, may be encapsulated within a semi-permeable barrier device and implanted into the affected animal, allowing passage of one or more therapeutic polypeptides encoded by the nucleic acid molecules in the conjugate or complex of the invention into the tissues and circulation of the animal, but preventing contact between the animal's immune system and the transfected cells (see WO 93/09222). These approaches result in increased production of one or more therapeutic polypeptides by the treated animal via (a) random insertion of the therapeutic gene (contained on the nucleic acid molecule of the invention) into the host cell genome; or (b) incorporation of the therapeutic gene into the nucleus of the cells where it may exist as an extrachromosomal genetic element. General descriptions of such methods and approaches to gene therapy may be found, for example, in U.S. Pat. No. 5,578,461; WO 94/12650; and WO 93/09222; the disclosures of all of which are incorporated herein by reference in their entireties.

Release of Nucleic Acids Intracellularly. Once internalized into a cell (typically via endocytosis), transfected nucleic acids are usually sequestered within lipid membrane-enclosed vesicles (including endosomes, as well as components of the endoplasmic reticulum (ER) and/or Golgi apparatus). The release of nucleic acids into the cytosol from endosomes, the ER or the Golgi enhances transfection. Endosomal disrupting agents can be used in the context of the invention and are defined herein as agents that cause or enhance the release of nucleic acids into the cytosol. Endosomal disrupting agents can act, by way of non-limiting example, by disrupting membranes of endosomes, the ER, the Golgi apparatus and/or other membranes; blocking or reducing endosome fusion to lysosomes; and/or altering, preferably raising, the pH of endosomes. The pH of an endosome is generally lower than that of the cytosol by one to two pH units. This pH gradient can be exploited for cellular delivery using agents that disrupt lipid bilayer membranes at pH 6.5 and below (Asokan A, Cho M J. 2002, Exploitation of intracellular pH gradients in the cellular delivery of macromolecules. J Pharm Sci 91:903-913).

Membrane-disruptive pH-sensitive synthetic polymers have been described and include by way of non-limiting example poly(amidoamine)s (PAAs) (Pattrick et al., 2001. Poly(amidoamine)-mediated intracytoplasmic delivery of ricin A-chain and gelonin. J Control Release 77:225-32; U.S. Pat. No. 6,413,941); poly(propylacrylic acid) (PPAA) (Kyriakides et al., 2002. pH-sensitive polymers that enhance intracellular drug delivery in vivo. J Control Release 78:295-303); and poly(ethyl acrylic acid) (PEAAc) (Murthy et al., 1999. The design and synthesis of polymers for eukaryotic membrane disruption. J Control Release 61:137-43).

Some cationic lipid transfection reagents, such as vectamidine and DMRIE-C, may have inherent endosomal disrupting properties. See El Ouahabi et al., 1997. The role of endosome destabilizing activity in the gene transfer process mediated by cationic lipids. FEBS Lett 414:187-92. Moreover, cationic lipids that are acid-labile have been described (Boomer et al., 2002. Formation of plasmid-based transfection complexes with an acid-labile cationic lipid: characterization of in vitro and in vivo gene transfer. Pharm Res 19:1292-1301; Wetzer et al., 2001. Reducible cationic lipids for gene transfer. Biochem J 356:747-756).

Other endosome disrupting agents include viral fusogenic peptides, including without limitation influenza virus hemagglutinin fusogenic peptides (Bongartz et al., 1994. Improved biological activity of antisense oligonucleotides conjugated to a fusogenic peptide. Nucleic Acids Res 22:4681-4688) and synthetic derivatives thereof (Plank et al., 1994. The influence of endosome-disruptive peptides on gene transfer using synthetic virus-like gene transfer systems. J. Biol. Chem. 269:12918-12924.) These peptides are thought to change conformation at acidic pH and destabilize endosomal membranes.

The ricin A chain, which is capable of penetrating out of endosomes and into the cytosol, can be attached to a nucleic acid or protein to in order to effect the endosomal release thereof (Beaumell et al., 1993. ATP-dependent translocation of ricin across the membrane of purified endosomes J. Biol. Chem. 268:23661-23669).

Agents that alter the pH of endosomes can be used to practice the invention. Lysosomotropic amines are generally thought to effect of raising the pH of endosomes. Such agents include without limitation ammonium chloride, 4-aminoquinolines (e.g., chloroquine, amodiaquine), 8-aminoquinolines (e.g., primaquine and WR242511), pyrimethamine, quinacrine, quinine and quinidine (Tsiang H, Superti F. Ammonium chloride and chloroquine inhibit rabies virus infection in neuroblastoma cells. Brief report. Arch Virol 81:377-382; Deshpande et al., 1997. Efficacy of certain quinolines as pharmacological antagonists in botulinum neurotoxin poisoning. Toxicon 35:433-445).

Artificial Chromosomes. The nucleic acid molecules used in the compositions, complexes and methods of the present invention may alternatively be in the form of artificial chromosomes (ACs). An AC is a DNA molecule that comprises, at a minimum, at least one origin of DNA replication (ori), one or more telomeres and a centromere. Each on is preferably derived from a genomic chromosome, so that replication of the AC is coordinated with cellular DNA replication. The telomeres are elements that preserve the terminal sequences of chromosomes for any number of rounds of replication and cell division. The centromere mediates proper segregation of the AC through each cell division (Willard H F. Centromeres: the missing link in the development of human artificial chromosomes. Curr Opin Genet Dev 8:219-225, 1998).

Ideally, ACs are stably maintained and are properly segregated during both mitosis and meiosis. Generally, an AC contains a segment of cloned DNA, and is usually more stable the larger the piece of cloned DNA. It is possible to engineer ACs to improve or add functions (Grimes B, Cooke H. Engineering mammalian chromosomes. Hum Mol Genet. 7:1635-1640, 1998; Saffery R, Choo K H. Strategies for engineering human chromosomes with therapeutic potential. J Gene Med 4:5-13, 2002).

Bacterial and yeast artificial chromosomes (BACs and YACs, respectively) have been described. BACs and YACs are reviewed in Shizuya H, Kouros-Mehr H. The development and applications of the bacterial artificial chromosome cloning system. Keio J Med 50:26-30, 2001; and Fabb S A, Ragoussis J. Yeast artificial chromosome vectors. Mol Cell Biol Hum Dis Ser 5:104-124, 1995; Anand R. Yeast artificial chromosomes (YACs) and the analysis of complex genomes, Trends Biotechnol 10:35-40, 1992.

Mammalian artificial chromosomes (MACs) have been prepared and may be used as vectors for somatic gene therapy. See Brown W R. Mammalian artificial chromosomes. Curr Opin Genet Dev 2:479-486, 1992; Huxley C. Mammalian artificial chromosomes and chromosome transgenics. Trends Genet. 13:345-347, 1997; Ascenzioni F, Donini P, Lipps H J. Mammalian artificial chromosomes—vectors for somatic gene therapy. Cancer Lett 118:135-142, 1997; Vos J M. Mammalian artificial chromosomes as tools for gene therapy. Curr Opin Genet Dev 8:351-359, 1998; and Vos J M. Therapeutic mammalian artificial episomal chromosomes. Curr Opin Mol Ther 1:204-215, 1999.

Human artificial chromosomes (HACs) have been described (Henning K A, Novotny E A, Compton S T, Guan X Y, Liu P P, Ashlock M A. Human artificial chromosomes generated by modification of a yeast artificial chromosome containing both human alpha satellite and single-copy DNA sequences. Proc Natl Acad Sci USA. 96:592-597, 1999; Larin Z, Mejia J E. Advances in human artificial chromosome technology. Trends Genet. 18:313-319, 2002). HACs include but are not limited to satellite DNA-based artificial chromosomes (SATACs). SATACs have been made by mixing human telomeric DNA, genomic DNA, and arrays of repetitive .alpha.-satellite DNA having centromeric activity (Hadlaczky G. Satellite DNA-based artificial chromosomes for use in gene therapy. Curr Opin Mol. Ther. 3:125-132, 2001).

In addition to gene therapy, ACs have been used to stably clone large pieces of DNA in a variety of cell types (Schlessinger D, Nagaraja R. Impact and implications of yeast and human artificial chromosomes. Ann Med 30:186-191, 1998; Monaco A P, Larin Z. YACs, BACs, PACs and MACs: artificial chromosomes as research tools. Trends Biotechnol. 12:280-286, 1994). In addition, ACs can be also be used in transgenic animal technologies to introduce large transgenes in animals, especially human transgenes in mouse models of human genetic diseases. See Giraldo P, Montoliu L. Size matters: use of YACs, BACs and PACs in transgenic animals. Transgenic Res 10:83-103, 2001; Jakobovits A, Lamb B T, Peterson K R. Production of transgenic mice with yeast artificial chromosomes. Methods Mol Biol 136:435-453, 2000; Lamb B T, Gearhart J D. YAC transgenics and the study of genetics and human disease. Curr Opin Genet Dev 5:342-348, 1995; Jakobovits A. YAC vectors. Humanizing the mouse genome. Curr Biol 4:761-763, 1994; Huxley C. Transfer of YACs to mammalian cells and transgenic mice. Genet Eng (NY) 16:65-91, 1994; Huxley C, Gnirke A. Transfer of yeast artificial chromosomes from yeast to mammalian cells. Bioessays 13:545-550, 1991; and Heintz N. BAC to the future: the use of bac transgenic mice for neuroscience research. Nat Rev Neurosci 2:861-870, 2001.

Peptide Nucleic Acids (PNAs). The nucleic acid molecules used in the delivery compositions, complexes and methods of the present invention may alternatively be in the form of peptide nucleic acids (PNAs). PNAs are analogs of nucleic acid molecules in which the backbone is a pseudopeptide rather than a sugar. Like DNA and RNA, a PNA molecule binds single-stranded nucleic acid having a reverse complementary sequence; however, the neutral backbone of PNAs can result in stronger binding and greater specificity. For a review, see Corey D R. Peptide nucleic acids: expanding the scope of nucleic acid recognition. Trends Biotechnol 15:224-229, 1997. The synthesis of PNAs is reviewed by Hyrup et al. (Peptide nucleic acids (PNA): synthesis, properties and potential applications. Bioorg Med. Chem. 4:5-23, 1996). For exemplary protocols for making and using PNAs, see Peptide Nucleic Acids: Protocols and Applications, Nielsen, P. E. and Egholm, M., eds. Horizon Scientific Press, Norfolk, U.K. 1999. PNAs can be prepared according to methods known in the art or purchased commercially from, e.g., Monomer Sciences Inc. (New Market, Ala., U.S.) and Dalton Chemical Laboratories Inc. (Toronto, ON, Canada). Methods for attaching fluorescent moieties to PNA have been described. See, e.g., Murakami et al., A novel method for detecting HIV-1 by non-radioactive in situ hybridization: application of a peptide nucleic acid probe and catalysed signal amplification. Pathol 194:130-135, 2001.

Polymer Synthesis and Polyplex Formation.

Materials. Diethylenetriaminepentacetic acid (DTPA) and ethyl trifluoroacetate were obtained from Alfa Aesar Chemical. All other reagents, and solvents used in the synthesis were obtained from Aldrich and were used without further purification. DTPA-bisanhydride (BA) was prepared using a standard procedure.

FIG. 1A, more particularly, provides schematic representations of two analogous polymeric imaging beacons that differ in the ethyleneamine length (3a or 3b contain 3 or 4 ethyleneamines, respectively). The structures can be chelated with either Eu³⁺ or Gd³⁺ for microscopy and MRI imaging, respectively. The design of these materials was inspired by the characteristics of successful cationic nucleic acid delivery vehicles and the many elegant macromolecular imaging agents being examined for disease diagnosis. Indeed, any metal, for examply lanthanides or transition metals capable of exhibiting an imaging functionality useful for any imaging modality including PET, SPECT, MRI, and Fluorescence Flash Luminescence imaging can be used. Preferred metals (depending on the particular application) may include those chosen from copper, manganese, iron, or a lanthanide metal chosen from lanthanum, cerium, praseodymium, neodymium, promethium, samarium, europium, gadolinium, terbium, dysprosium, holmium, erbium, thulium, ytterbium, and lutetium to name a few.

The amine-containing co-monomer precursors were synthesized using a series of protection/deprotection reactions previously published by the inventors shown in Scheme S1

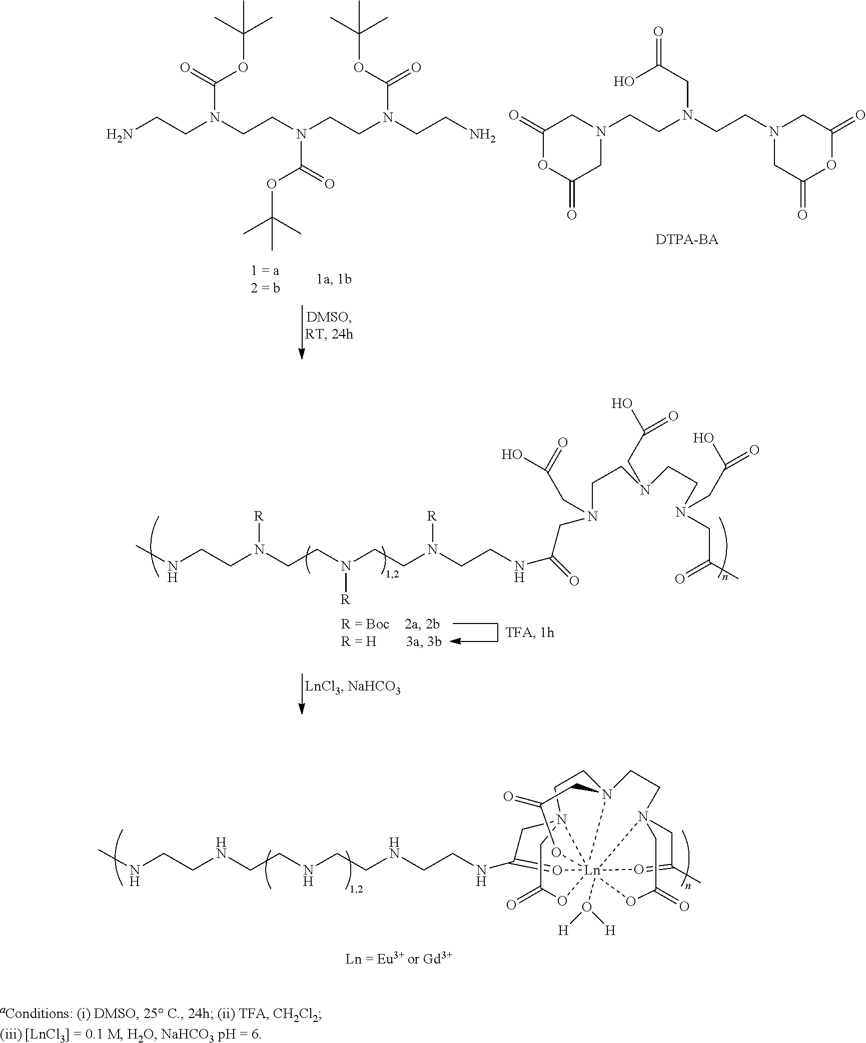

Synthesis according to Scheme 1 of monomers 1a and 1b was performed according to a previously published procedure developed by the inventors. See Srinivasachari & Reineke (2006) above. Next, DTPA-BA was reacted with 1a or 1b in dimethyl sulfoxide at room temperature for 24 h to form two polymer structures 2a and 2b (Scheme 1) with protected oligoethyleneamine units and pendant carboxylates from the anhydride ring opening during polymerization. The Boc protecting groups were removed yielding 3a and 3b and confirmed by NMR analysis.

More generally, synthesis can be performed according to the following scheme, which outlines a procedure for synthesizing the polymers using any desired metal:

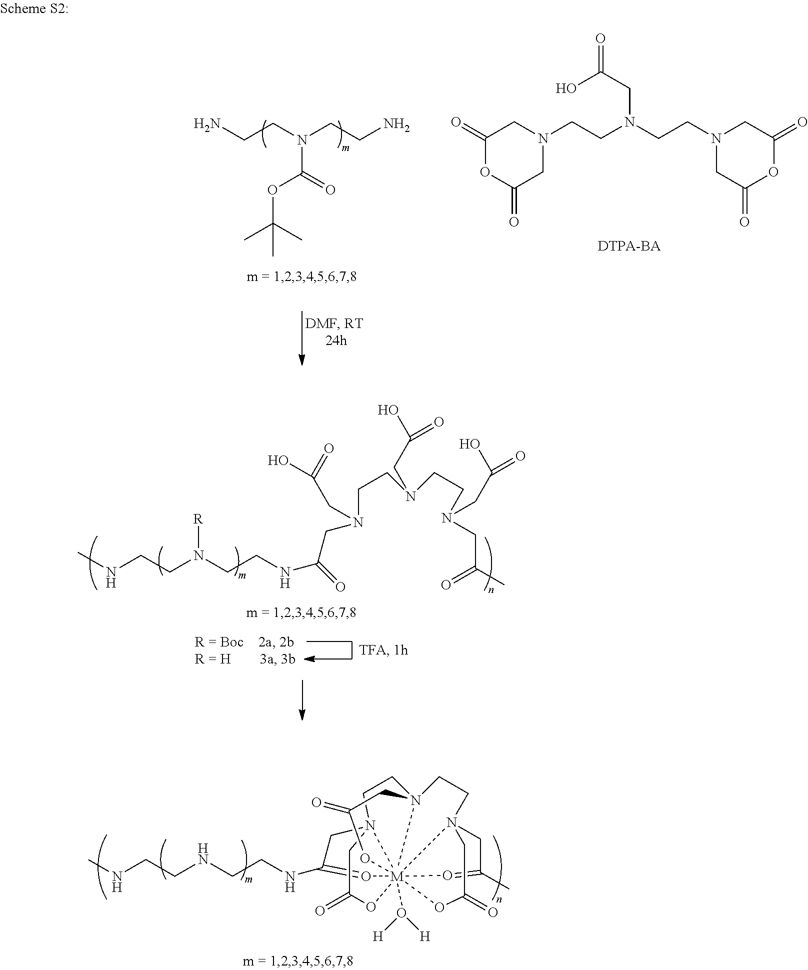

wherein R is a Boc (t-butoxycarbonyl) group or a hydrogen atom.

FIG. 1B provides a schematic diagram of general exemplary polymeric imaging beacons according to embodiments of the invention, which can be produced using Scheme S2. Polymeric imaging beacons according to the invention can comprise repeating units (as shown in FIG. 1B) of metal chelates within an oligoamine backbone, wherein n is an integer ranging from from 2 to 10,000,000; M is a metal capable of exhibiting an imaging functionality for an imaging modality; and the oligoamine backbone comprises from 1 to 8 ethlyeneamines and is chosen from diethyleneamine, triethylenediamine, tetraethylenetriamine, pentaethylenetetraamine, hexaethylenepentamine, heptaethylenehexamine, octaethyleneheptamine, or nonethyleneoctaamine.

FIG. 1C provides a schematic diagram of unchelated polymers according to embodiments of the present invention. As shown, the invention includes the unchelated polymers having an original repeating unit of the structure shown in FIG. 1C, having a positive charge, and capable of binding with one or more functional group or metal, wherein R is chosen from a hydrogen atom, or a methyl or t-butoxycarbonyl (Boc) group; and n ranges from 2 to 10,000,000. Several synthesis examples include:

### Example 1

### Synthesis of Polymer 2a: Poly([N2,N3,N4-tris(tert-butoxycarbonyl)-tetraethylene-triamine]amidodiethylenetriaminetriaacetic acid)

Polymer 2a was prepared by dissolving monomer 1a (3.63 g, 7.41 mmol) in 25 mL of DMSO at room temperature. DTPA-BA (2.64 g, 7.41 mmol) was dissolved in 5 mL of DMSO and added directly to the solution of 1a under constant stirring. The polymerization was carried out for 18 h, after which the solution was pipetted into a 6,000- to 8,000-DaMWCO dialysis bag (Spectrum laboratories) and dialyzed extensively against methanol for 18 h, after which the solution was removed from the bag and evaporated in vacuo to yield 2a as an orange viscous oil (yield=3.88 g, 47%).

### Example II

### Synthesis of Polymer 2b: Poly([N2,N3,N4,N5-tetrakis(tert-butoxy-carbonyl)pentaethylenetetramine]amidodiethylenetriaminetriaacetic acid)

Polymer 2b was prepared according to a procedure identical to that of synthesis of Polymer 2a, except monomer 1b (4.77 g, 7.54 mmol) was polymerized with DTPA-BA (2.69 g, 7.54 mmol) yielding a deep amber oil (yield=4.17 g, 56%).

### Example III

### General Synthesis of Polymers 3a and 3b: Poly[(tetraethylene-triamine)amidodiethylenetriaminetriaacetic acid] and Poly[(pentaethylenetetramine)-amidodiethylenetriaminetriaacetic acid]

An aliquot of 5 mmol of each protected polymer (2a or 2b, respectively) was dissolved in 20 mL of CH₂Cl₂ and cooled to 0° C. Next, 20 mL of trifluoroacetic acid was added to each solution, and the reaction was warmed to room temperature. The reactions were allowed to proceed for 3 h, after which the solvents were removed in vacuo and each mixture was redissolved in water. Both water solutions were brought to a pH=6 using a 1 M sodium bicarbonate. The solutions were each deposited in a separate 6,000- to 8,000-Da MWCO dialysis bag (Spectrum laboratories) and dialyzed against ultrapure water for 24 h. The dialyzed solutions were lyophilized to yield the fluffy, unchelated polymers 3a and 3b (yields=1.88 g, 76%; 1.97 g, 80%, respectively).

NMR spectra were acquired on a Bruker AV-400 MHz spectrometer. ₁H NMR data are reported as follows: chemical shift (δ ppm), multiplicity (s=singlet, d=doublet, t=triplet, q=quartet, m=multiplet, bs=broad singlet), and the peak integration. Polymers were dissolved and diluted to the ppb range so they could be analyzed within the proper calibration curve range. The analytical data are shown in Table 1.

TABLE 1
₁H and ₁₃C NMR Data for Polymers 2a & 2b

| Polymer | | ₁H-NMR (d₃-MeOD) | ₁₃C-NMR (d₃-MeOD) |
|---|---|---|---|
| 2a | Poly{[N₂,N₃,N₄-tris(tert-butoxy- | δ = 1.57 (s, 27H), | δ = 30.21, 37.42, 40.87, |
| | carbonyl) tetraethylenetriamine]-amido- | 3.08 (s, 4H), | 44.33, 46.03, 47.10, |
| | diethylenetriaminetriaacetic acid} | 3.30 (bm, 20H), | 49.02, 50.94, 52.63, |
| | | 3.43 (bs, 6H), | 55.52, 57.64, 79.14, |
| | | 3.69 (s, 4h) | 154.95, 155.14, 169.77, |
| | | | 170.89, 173.02 |
| 2b | Poly{[N₂,N₃,N₄,N₅-tetrakis(tert- | δ = 1.57 (s, 27H), | δ = 28.49, 37.27, 37.79, |
| | butoxycarbonyl)pentaethylenetetramine] | 3.10 (s, 8H), | 40.87, 44.33, 45.04, |
| | amidodiethylenetriaminetriaacetic acid} | 3.28-3.53 (bm, 20H), | 45.96, 46.98, 49.02, |
| | | 3.43 (bs, 6H), | 50.94, 52.63, 55.52, |
| | | 3.69 (s, 4H) | 57.64, 79.14, 154.95, |
| | | | 155.14, 169.77, 170.89, |
| | | | 173.02 |

| | | |
|---|---|---|
| Polymer | ₁H-NMR (D₂O): | ₁₃C NMR (D₂O): |

| | | | |
|---|---|---|---|
| 3a | Poly-tetraethylenepentaamineamido- | δ = 3.08 (s, 4H), | δ = 35.67, 43.90, 44.08, |
| | diethylenetriaminetetraacetic acid | 3.24-3.50 (bm, 20H), | 47.56, 50.93, 53.43, |
| | | 2.54 (bs, 6H), | 55.04, 58.72, 171.61, |
| | | 3.77 (s, 4H) | 173.31, 177.64 |
| 3b | Poly-pentaethylenehexamineamido- | δ = 3.03 (s, 4H), | δ = 35.74, 43.86, 44.28, |
| | diethylenetriaminetetraacetic acid | 3.21-3.49 (bm, 20H), | 45.11, 47.34, 50.88, |
| | | 2.54 (bs, 6H), | 53.39, 55.00, 58.72, |
| | | 3.77 (s, 4H) | 171.59, 173.29, 177.67 |

The deprotected precursors were then chelated as described in Example IV.

### Example IV

### General Synthesis of Chelated Polymers 3a and 3b: Poly[(tetra-ethylenetriamine)amido(Ln³⁺)diethylenetriaminetriacetate], Eu3a or Gd3a, and Poly[(penta-ethylenetetramine)amido(Ln³⁺)diethylenetriaminetriacetate], Eu3b or Gd3b

A 2-mmol aliquot of each unchelated polymer, 3a and 3b, was dissolved in 20 mL of ultrapure water at room temp. Next, 2 mmol of LnCl₃ (either EuCl₃ or GdCl₃) was dissolved in 5 mL of water and dripped into the respective polymer solution in three separate aliquots. The pH was adjusted to pH=6 after each aliquot addition. The solution was allowed to stir for 2 h and then was dialyzed in a 6,000- to 8,000-DaMWCO bag against ultrapure water for 24 h. Dialyzed solutions were lyophilized to yield fluffy off-white polymers Gd3a, Gd3b, Eu3a, and Eu3b (yields=Gd3a=1.20 g, 86%; Gd3b=1.19 g, 80%; Eu3a=1.13 g, 81%; Eu3b=1.17 g, 79%). The compounds obtained by chelating the precursors with the chloride salts of Eu³⁺ and Gd³⁺ were confirmed by FT-IR and ICP-MS. These results are shown in Tables 2 and 3.

TABLE 2
FT-IR Data for the Polymers*

| Polymer | ν, cm⁻¹ |
|---|---|
| 2a | 1,663.3; 1,712.3; 3,334.2 |
| 2b | 1,666.3; 1,692.9; 3,346.3 |
| 3a | 1,641.2; 1,692.9; 3,415.2 |
| 3b | 1,650.8; 1,667.8; 3,419.2 |
| Eu3a | 1,585.4; 1,634.5; 3,419.4 |
| Eu3b | 1,585.2; 1,633.8; 3,419.9 |
| Gd3a | 1,587.2; 1,641.2; 3,433.7 |
| Gd3b | 1,586.1; 1,633.9; 3,419.6 |

*Samples of each polymer (5 mg) were crushed by mortar and pestle with 20 mg of anhydrous KBr and compacted into a translucent pellet. Spectra were measured on a Perkin-Elmer Spectrum One Fourier transform infrared spectrometer.

It should be mentioned that neither ₁H nor ₁₃C NMR data could be collected for Eu3a, Eu3b, Gd3a, and Gd3b, due to the paramagnetic nature of these materials. Mass spectra were obtained with an IonSpec ESI mass spectrometer in positive ion mode. IR spectra were measured on a Perkin Elmer Spectrum One Fourier transform infrared spectrometer as KBr pellets. Lanthanide quantification was carried out on a ICP-MS (Octapoler detection, Agilent Technologies, Santa Clara, Calif.), which monitored Gd isotopes (157, 158 Da) and Eu isotopes (151, 153 Da). These data were used to generate calibration curves for both Gd³⁺ and Eu³⁺.

TABLE 3
Ln Quantification for the Polymer Series*

| | Calculated Ln | Observed Ln |
|---|---|---|
| Polymer | Content, % | Content, % |
| Eu3a | 21.8 | 21.8 |
| Eu3b | 20.5 | 20.4 |
| Gd3a | 22.5 | 22.8 |
| Gd3b | 21.1 | 21.9 |

*The percentage of Ln by mass was determined by diluting polymer samples to the ppb range and analyzed by inductively coupled plasma MS. For each polymer, the signal integration was fitted to calibration curves generated from Ln standards.

The final Ln-chelated polymers were analyzed by GPC using a triple detection system (refractive index, static light scattering, and viscometry). The molecular mass and polydispersity for polymers 3a, 3b, Eu3a, Eu3b, Gd3a, and Gd3b were measured with a Viscotek GPCmax instrument equipped with a GMPWXL (aqueous phase) column coupled to a triple detection system (static light scattering, viscometry, and refractive index). A solution of 0.5M sodium acetate containing 20% acetonitrile was used as the mobile phase. As shown in Table 4, a similar molecular mass, degree of polymerization, and polydispersity were achieved for all polymer structures 3a, 3b, Eu3a, Eu3b Gd3a, and Gd3b, which was expected because all of the Ln-containing polymers were synthesized from the same parent batches of 3a and 3b.

TABLE 4
Weight Averaged Molecular Weight (M_{w}), Polydispersity
(M_{w}/Mₙ), and Degree of Polymerization (n_{W}) for the Polymers*

| Polymer | M_{w}, kDa | M_{w}/Mₙ | n_{W} |
|---|---|---|---|
| 3a | 43 | 1.7 | 78 |
| 3b | 54 | 1.9 | 91 |
| Eu3a | 64 | 1.7 | 91 |
| Eu3b | 68 | 1.7 | 92 |
| Gd3a | 67 | 1.9 | 96 |
| Gd3b | 62 | 2.0 | 89 |

*Due to similarities in polymer characteristics and similar chemical properties of lanthanides in the 3+ oxidation state, sound comparison of polymer biological activity was possible between different analogs.

### Example V

Polymer Ability to Complex pDNA. The polymers, 3a, 3b, Eu3a, Eu3b, Gd3a, and Gd3b, were examined for their ability to complex pDNA using an electrophoretic gel shift assay.

Nuclease Degradation Assay. The ability of the polymers to protect pDNA from nuclease degradation was analyzed according to a modified form of a previously published procedure. See Liu Y, Reineke T M (2006) Poly(glycoamidoamine)s for gene delivery: Stability of polyplexes and efficacy with cardiomyoblast cells, Bioconjugate Chem 17:101-108, the disclosure of which is incorporated herein by reference in its entirety.

Generally, polyplexes were formulated at N/P ratios (N=polymer secondary amine no.; P=DNA phosphate no.) between 0 and 40 before being electrophoresed in an agarose gel. Polymers Eu3a, Eu3b, Gd3a, and Gd3b began to charge-neutralize pDNA at an N/P ratio of 5, and pDNA migration was mostly suppressed at N/P of 20. With these polymers, the N/P ratio needed to fully inhibit gel migration of pDNA was higher than other polyamidoamine vehicles created by the inventors previously. See Srinivasachari and Reineke (2006) above.

It is interesting to note that the non-chelated polymers (3a and 3b) do not bind with pDNA even at an N/P ratio of 100. This result is likely due to the fact that the non-chelated polymers have negatively-charged carboxylates, which likely neutralizes the positive charge (or otherwise lowers the cationic charge) from the amine groups on the polymer backbone.

More particularly, each polymer (5 μL) was combined with pCMVβ (1 μg in 5 μL of water) to form polyplexes at N/P=40. After 30 min, FBS (5 μL) was added to each polyplex solution, and the samples were incubated for 0, 1, 2, 4, and 6 h, at 37° C. The samples were then treated with 10% SDS (2 μL), and then stored at 4° C. until all samples were finished incubating. All trials were then incubated at 60° C. for 18 h to release the polymer from the pDNA. Loading buffer (2 μL, Blue Juice; Invitrogen) was added to an aliquot of each polyplex solution and loaded onto an agarose gel (0.6%, 3 μL ethidium bromide) in 20 μL aliquots, and electrophoresed to analyze for plasmid degradation. Plasmid DNA only and FBS were used as negative controls.

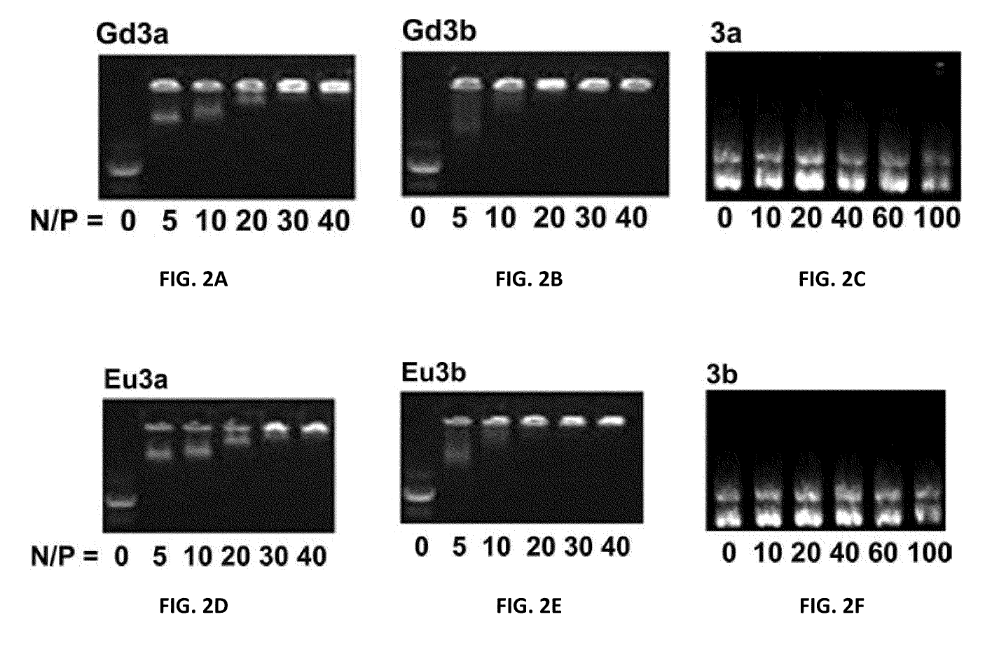

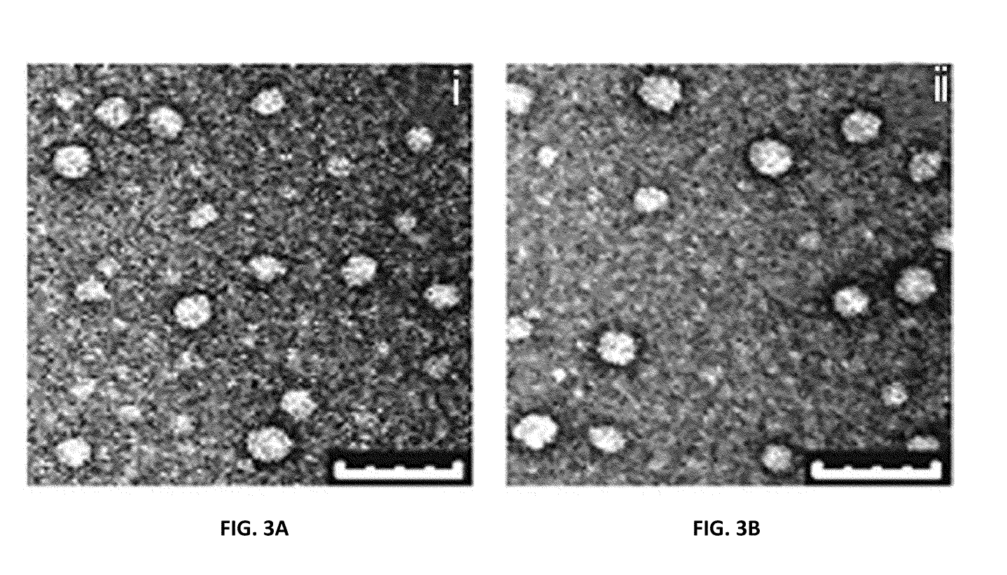

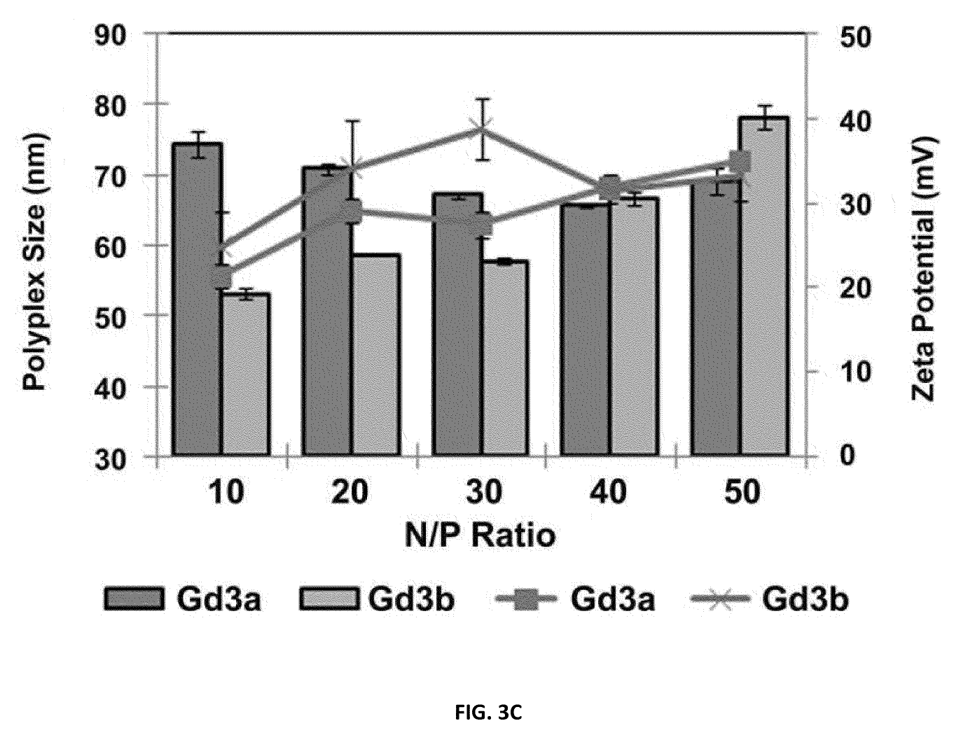

As shown in FIGS. 2A-F, Agarose gel electrophoresis shift assays allow observation of Gd3a (FIG. 2A), Gd3b (FIG. 2B), Eu3a (FIG. 2D), and Eu3b (FIG. 2E) binding with pDNA at increasing N/P ratios from 0 to 40. Hindrance of pDNA migration is noted with all polymers at N/P=5; however, migration is not completely hindered until N/P=30 for Eu3a and Gd3a and N/P=20 for Gd3b and Eu3b. Non-chelated polymers 3a (FIG. 2C) and 3b (FIG. 2F) do not hinder to pDNA migration in agarose gel and, thus, do not bind pDNA.

The size and morphology of the polyplexes were then examined using transmission electron microscopy (TEM) and dynamic light scattering (DLS). TEM studies (Philips EM 420 Scanning TEM) were performed according to a previously published method. See Srinivasachari & Reineke (2006) above. In particular, polyplex sizes and zeta potential were measured using a Zetasizer (Nano ZS) DLS instrument with a 633 nm laser (Malvern Instruments). Polyplexes were formed at N/P ratios at 10, 20, 30, 40, and 50 by combining equal volumes of a polymer solution in ultrapure water with a 0.02 mg mL⁻¹ pDNA solution and allowing the polyplexes to form for 1 h.

FIGS. 3A-C, show polyplex size and charge as observed by transmission electron microscopy, dynamic light scattering, and zeta potential measurements. FIG. 3A shows a TEM micrograph of polyplex Eu3a and FIG. 3B shows a TEM micrograph for polyplex Eu3b, where for both N/P=40). Polyplexes were negatively stained with uranyl acetate (Scale bar, 100 nm.) and as shown in FIG. 3C, DLS (bars) and zeta potentials (lines) of polyplexes at various N/P ratios (the average and SD of three measurements are shown). TEM micrographs indicate that the polyplexes formulated with Eu3a and Eu3b and pDNA at N/P of 40 exhibit spherical morphology with particle diameters between 35 and 60 nm (FIGS. 3A and 3B).

As shown in FIG. 3C, DLS and zeta potential data generally showed slightly larger particle sizes resulting from measuring the hydrodynamic radii (sphere of hydration). In the DLS studies, the size of Gd3a polyplexes slightly decreased with the N/P ratio (from 74 to 66 nm); however, Gd3b polyplexes generally increased in size with the N/P ratio (from 53 to 78 nm). Zeta potential measurements reveal that polyplexes exhibited a positive surface charge (between 20 and 40 mV) that increased with the N/P ratio. The inventors were unable to analyze polyplexes formed with Eu3a and Eu3b by DLS and zeta potential measurements due to laser/detector interference with the absorption/luminescence emission bands of Eu³⁺.

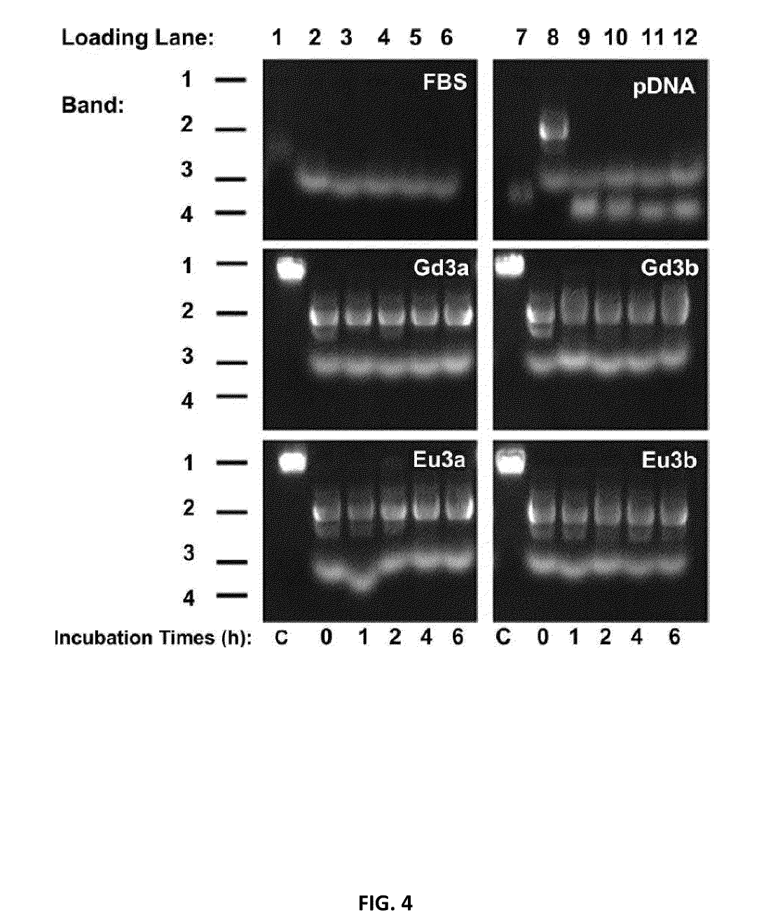

The stability of these polyplexes from nuclease degradation was examined by exposure to high concentrations of FBS (33%), and pDNA integrity was observed using gel electrophoresis. These data show there is no evidence of pDNA degradation in the polyplexes at any time point assayed (shown as the lack of formation of band 4 (FIG. 4) Gels Eu3a, Eu3b, Gd3a, and Gd3b). Also, the brightness of the pDNA band in the polyplex gels (band 2, FIG. 4) remains constant as a function of exposure time. In the control gel of naked pDNA (gel pDNA, lanes 9-12), band 2 was absent, and band 4 (degraded DNA) was clearly observed, indicating full degradation after only 1 h of incubation in 25% FBS.

### Example VI

Cellular Delivery and Toxicity Studies. Toxicity assays were conducted to assess the cellular uptake, toxicity, and transfection efficiency of the polyplexes formulated with the polymer beacons. HeLa cells were transfected with polyplexes formulated with Cy5-pDNA and either Eu3a, Eu3b, Gd3a, or Gd3b. In particular, twenty-four hours before transfection, HeLa cells were seeded in six well plates at a density of 250,000 cells per well and incubated in supplemented DMEM (10% FBS) at 37° C. and 5% CO₂. Polyplexes were formulated by adding 250 μL solutions of each polymer or control dissolved in water (concentration calculated based on N/P ratios of 40 and 60) to 5 μg of Cy5-labeled pDNA (250 μL solution). Just before transfection, 1 mL Opti-MEM was added to each polyplex solution. Cells were aspirated of old media, washed with 1 mL PBS, and the appropriate polyplex solution was added to each well. Cells were then incubated at 37° C. under a 5% CO₂ atmosphere for 4 h.

The cell suspensions were then prepared for analysis as previously described. See Srinivasachari & Reineke (2006) above. Generally, four hours after transfection, cellular uptake efficiency was determined by monitoring the fluorescence intensity of Cy5-labeled pDNA using flow cytometry. Cell viability experiments were conducted by assaying the cells for protein content 48 h after transfection. Toxicity was assessed by normalizing the results of the protein assay to a control of untreated cells; this assay was then used to calculate the fraction of viable cells in each well. The transfection efficiency was also monitored 48 h after transfection using a luciferase reporter gene expression assay.

HeLa cell suspensions were analyzed on a FACS Canto II flow cytometer equipped with a 633 nm helium-neon laser. MeanCy5 fluorescence intensity was measured using the appropriate forward and side scatter gates. A control of untransfected cells was used to create a gate such that less than 1% of cell-associated autofluorescence is detected in the Cy5 channel. This gating strategy was used for subsequent samples of transfected cells to determine the percentage of cells transfected. For gene expression analysis, polyplexes were formulated in an identical manner as above except with GWiz-lucpDNA and allowed to transfect cells for 48 h before assaying for luciferase activity according to previously published methods. Cell viability was characterized by measuring the amount of protein in cell lysates using a Bio-Rad DC protein assay kit in triplicate. Viability is reported as the fraction of protein in each sample normalized to a control of untransfected cells.

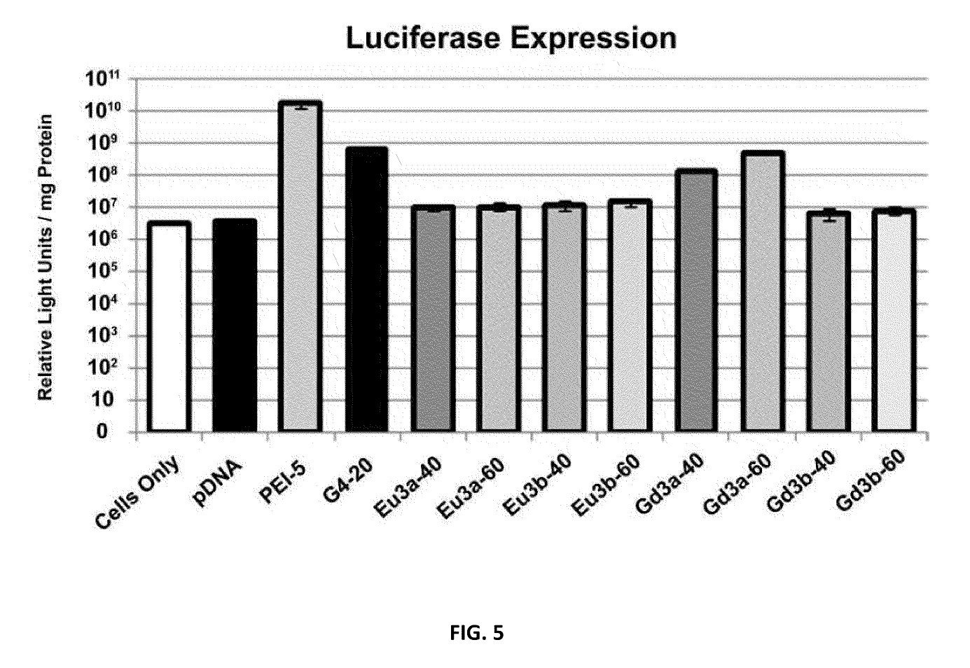

FIG. 5 shows the results of Luciferase Expression in HeLa cells. The N/P ratio of the polyplex used is indicated after the polymer name on the x axis. Polyplexes were formed using the same methodology as the DLS studies. The results are reported as relative light units (RLUs) emitted by the catalyzed transformation of luciferin per milligram of protein. It should be mentioned that these conventional assays only yield pDNA uptake and transcription efficiency, but do no yield information about the fate of the polymer (thus, the development and study of our intracellular delivery beacons; see below).

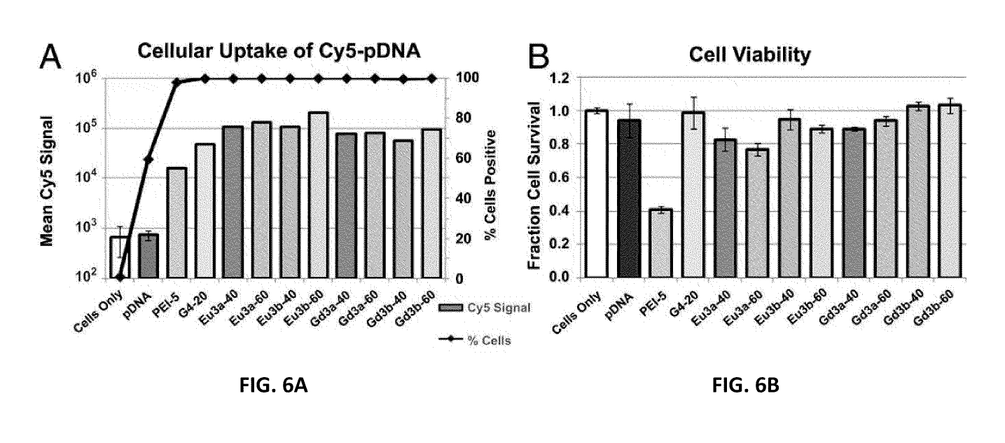

All polyplexes formed with these polymers promoted high cellular uptake while maintaining high viability. As shown in FIGS. 6A and 6B, the effect of lanthanide chelate and N/P ratio on polyplex uptake into Hela cells and cell viability is demonstrated.

FIG. 6A shows cellular uptake of polyplexes formulated using Cy5-pDNA. The percentage of cells (line) containing Cy5pDNA and mean fluorescence intensity (bar) of Cy5 in a population of at least 30,000 cells. FIG. 6B shows cell viability after exposure to polyplexes using unlabeled pDNA. The N/P ratio of the polyplex used is indicated after the polymer name on the x axis. The negative controls consisted of cells only and pDNA only, whereas positive controls consisted of polyplexes formed with Jet-PEI at an N/P=5 and polyplexes formed with G4 at N/P of 20. Polymer G4 is a polyamidoamine delivery vehicle previously developed by the inventors that consists of alternating meso-galactaramide units and four ethyleneamine groups. See Liu and Reineke (2005) above. The delivery beacons yielded high pDNA uptake (approximately 100% of cells), and the intensity of Cy5 fluorescence was higher than that of the positive controls, Jet-PEI and G4, indicating that these vehicles are promising for further studies.

In FIG. 6B, toxicity of these structures is very low; even at very high polymer concentrations (high N/P ratios), cell viability remained very high (between 80 and 100% cell viability) in contrast with the positive control Jet-PEI (cell viability only 40% at low N/P=5).

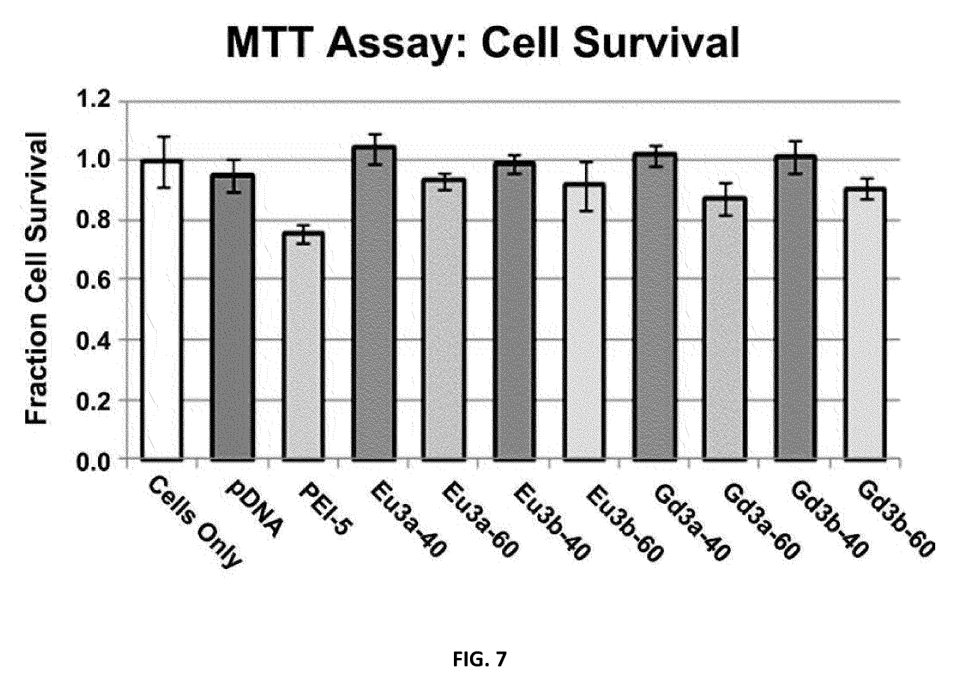

As shown in FIG. 7, similar high cell viability (greater than 80%) is verified when studied by an MTT assay with cells cultured under the same conditions. More specifically, FIG. 7 is a graph showing cell viability after exposure to polyplexes using unlabeled pDNA. Viability is reported as a measure of the MTT conversion normalized to untreated cells. The N/P ratio of the polyplex used is indicated after the polymer name on the x axis. Again, polyplexes were formed using the same methodology as the DLS studies.

Comparing these results to that of the luciferase expression shown in FIG. 5, these systems displayed slightly lower gene expression (107-108 RLU/mg) compared with that of the positive controls, G4 (109 RLU/mg) and Jet-PEI (1010 RLU/mg). However, as previously discussed, these polymer vehicles yielded higher cellular uptake than both positive controls and much lower toxicity than Jet-PEI. See, e.g., Chollet P, Favrot M C, Hurbin A, Coll, J-L (2002) Side-effects of a systemic injection of linear polyethylenimine-DNA complexes, J Gene Med 4:84-91; and Wightman L, et al. (2001) Different behavior of branched and linear polyethylenimine for gene delivery in vitro and in vivo, J Gene Med 3:362-372.

Although the small discrepancy between the uptake and gene expression data is not currently fully understand, the imaging experiments (see below) indicate that a much higher fraction of the internalized polyplexes are located in the cytoplasm, whereas a lower fraction is found in the nucleus. Nuclear entry may be the main barrier for gene expression. For many forms of nucleic acid therapeutics, such as siRNA, high cytoplasmic delivery is the ultimate goal. These delivery vehicles could be extremely useful in these therapeutic methodologies.

### Example VII

Cellular Imaging of the Polymer Beacons. Further investigation of the polymer beacons chelated with luminescent Eu³⁺ was examined using fluorescence microscopy. HeLa cells were transfected with polyplexes formulated with FITC-labeled pDNA and either Eu3a or Eu3b at an N/P ratio of 40.

Fluorescence Microscopy.

Twenty-four hours before transfection, cells were seeded in six well plates containing 25 mm no. 1 glass coverslips at a density of 50,000 cells per well. Just before transfection, polyplexes were formed as noted above for the cellular uptake assays except 200 μL of polymer solution was added to 200 μL of FITC-labeled pDNA ([pDNA]=0.02 mg/mL; N/P=40). Cells were removed from the incubator, aspirated of old media, washed with 2 mL PBS, and 2 mL Opti-MEM was added to each well. To transfect cells, 1 mL Opti-MEM from each well was added to the appropriate polyplex solution, pipetted up and down to mix, and returned to the well to deliver a total of 4 μg DNA per well.

Four hours after transfection, 2.4 mL of supplemented DMEM was added to each well. Twenty-four hours after transfection, cells were aspirated of old media, washed three times with 1 mL PBS, and fixed for 2 h using 2% paraformaldehyde in PBS (pH 7.4) at 4° C. Then, coverslips were removed from each well and carefully washed four times with 0.5 mL PBS. Coverslips were then mounted in Prolong antifade mounting media (Molecular Probes) and allowed to dry at room temperature overnight.

Cells were observed using a Zeiss Axioplan Imaging 2 infinity-corrected, upright scope, a 63× oil objective (N.A. 1.4), and standard filter sets for FITC and Rhodamine. To visualize Europium luminescence, a custom filter set (excitation max 405 nm±40 nm, dichroic 440 nm LP, emission max 610±75 nm) was built from filters purchased from Chroma. Images of each cell were collected as z-stacks with a z-spacing of 0.27 μm (Eu3a and Eu3b) using an Orca-ER CCD camera (Hamamatsu). The resulting images were processed using AutoQuant Autodeblur (Media Cybernetics). Data correction for each z-stack was applied for bias and flatfield frame and optical density. Then, stacks were processed using 3D blind deconvolution over 50 iterations. The final fluorescence images were minimally processed for background subtraction, brightness, and contrast. To increase fine detail in some DIC images, filters for pseudoflatfield and kalman stack were applied. After deconvolution, all image processing was completed using ImageJ open source software (National Institutes of Health). See Rasband WS (1997-2007) IMAGE J (National Institutes of Health, Bethesda).

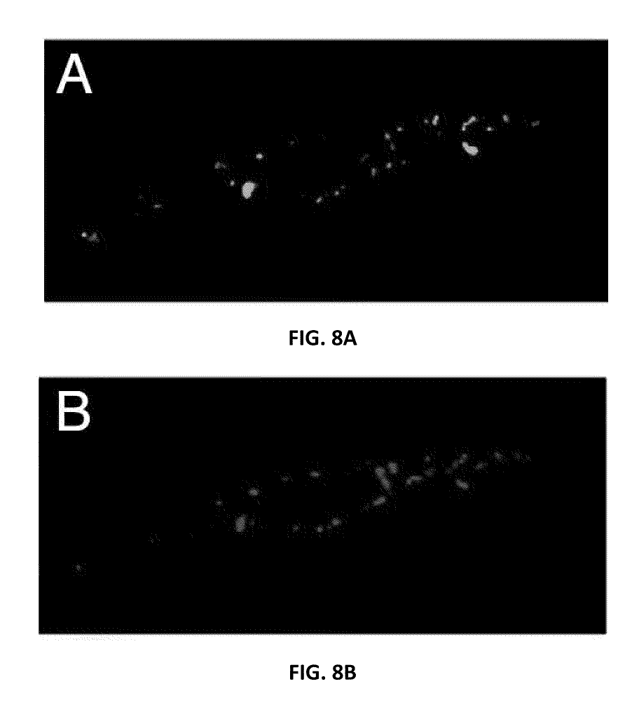

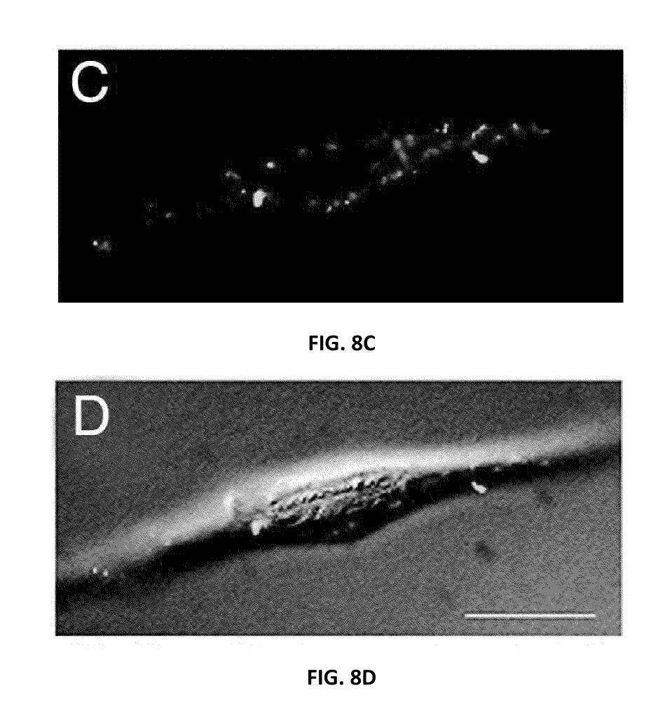

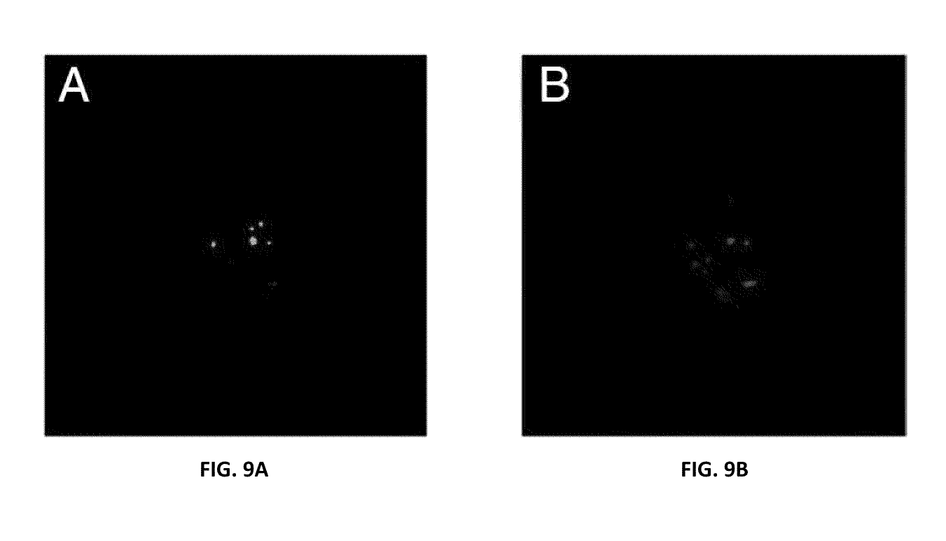

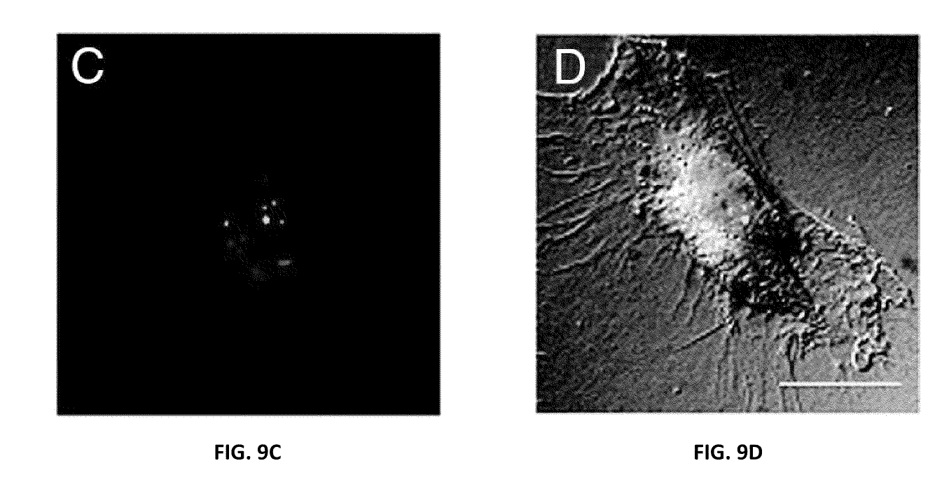

FIGS. 8A-D and FIGS. 9A-D each represent one vertical optical slice obtained by deconvolution of a Z-stack series. In particular, FIGS. 8A-D show deconvoluted micrographs of a HeLa cell transfected with FITCpDNA/Eu3a polyplexes. FIG. 8A is the FITC-pDNA fluorescence (green). FIG. 8B is the Eu³⁺ luminescence within the Eu3a beacons (red). FIG. 8C is an overlay of the FITC-pDNA and Eu3a images. Yellow pixels can be qualitatively used to visualize regions of colocalization. FIG. 8D is an overlay of the FIG. 8C image with a DIC image to show contrast of the cell. (Scale bar, 20 μm.).

FIGS. 9A-D are deconvoluted micrographs of a HeLa cell transfected with FITCpDNA/Eu3b polyplexes. FIG. 9A is FITC-pDNA fluorescence (green). FIG. 9B is Eu³⁺ luminescence within the Eu3b beacons (red). FIG. 9C is an overlay of the FITC-pDNA and Eu3b images. Yellow pixels can be qualitatively used to visualize regions of co-localization. FIG. 9D is an overlay of FIG. 9C and a DIC image to show contrast of the cell. (Scale bar 20 μm.)

The intracellular location of both FITC-pDNA (FIGS. 8A and 9A) and the polymers (FIGS. 9A and 9B) can be clearly observed. By overlaying the pDNA and polymer images (FIGS. 8C and 9C), co-localization can be qualitatively observed by yellow punctate staining. Most of the staining resulting from FITC-pDNA or Eu³⁺-polymer luminescence is localized to the cytoplasm and a lower fraction appears in the nucleus. As previously stated, this observation could be a contributing factor to the lower luciferase gene expression, but could prove useful in the development of cytoplasmic-targeted nucleic acid therapies. Also, it is clear that a small amount of uncomplexed pDNA is present within the cells whereas a larger fraction of free polymer is observed in the cellular cytoplasm.

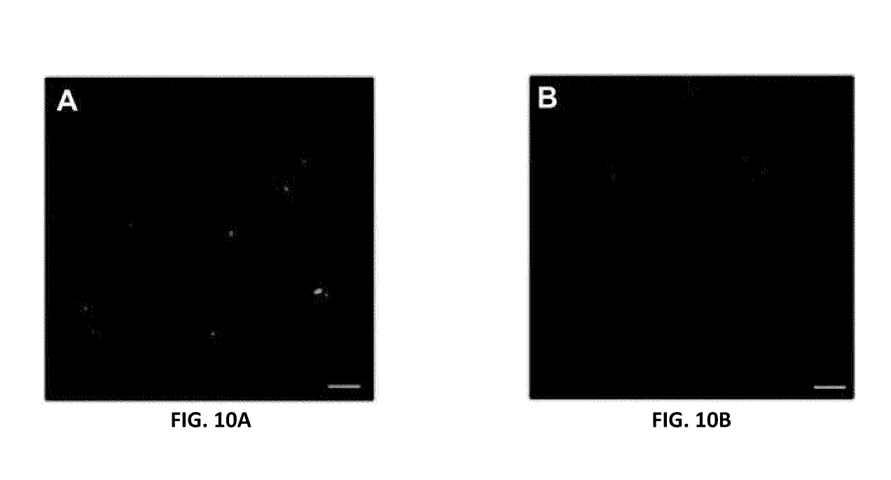

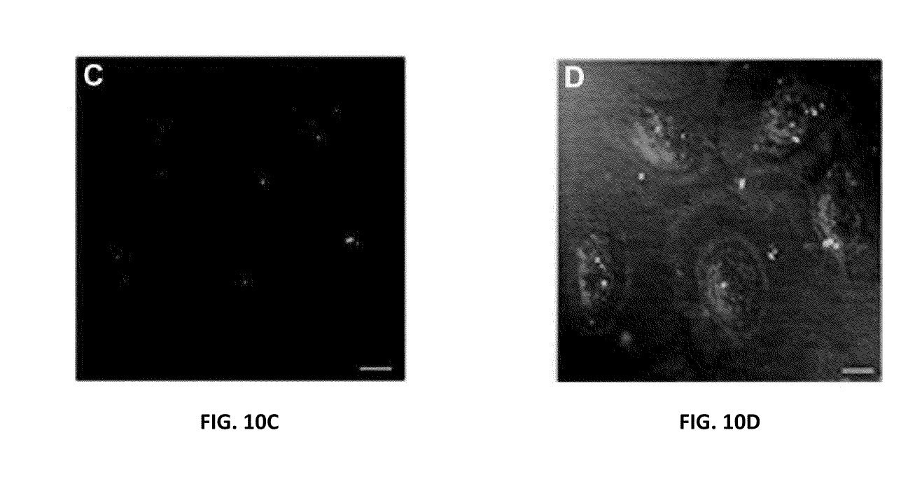

As shown in FIGS. 10A-D, the inventors have observed a similar result by formulating polyplexes at a lower N/P ratio of 20, where a high amount of free polymer was also observed in the cells. More specifically, FIGS. 10A-D show two-photon confocal images of FITC-labeled pDNA delivered with Eu3a at an N/P of 20. Infrared red laser was tuned to 780 nm to initiate Eu(III) excitation. Slice thickness was 1.2 μm, 1.2 μm pinhole, 63× oil-immersion objective, numerical aperture 1.4. (Scale bar, 20 μm.) In particular, FIG. 10A is the FITC-pDNA fluorescence; FIG. 10B is Eu3a luminescence; FIG. 10C is an FITC/Eu3a overlay; and FIG. 10D is an FITC/Eu/DIC overlay. This image indicates that diffuse cytoplasm staining of Eu3a is similar at lower N/P ratios.

These phenomena could be attributed to the excess used to formulate polyplexes at high N/P ratios, but may indicate premature pDNA release during cellular uptake and early intracellular trafficking (both are concerns in this area, but not well understood). Also, for Eu3a, the intracellular staining pattern is more punctate, whereas diffuse staining is more apparent for Eu3b. These data could indicate that Eu3a is more localized to endocytic vesicles, whereas Eu3b (with more secondary amines per repeat unit) could promote higher endocytic release. These data reveal that the polymer beacons evaluated herein (and future derivatives) will be useful in understanding intracellular polymer fate, as well as elucidating the mechanisms of delivery.

Inversion Recovery Experiments of Polyplexes in Solution. T1 measurements were carried out on Bruker AMX-400 MHz (9.4 T) and Anasazi FT-NMR 60 MHz (1.41 T) NMR spectrometers for all samples. Polyplexes were formed at an N/P=40 such that the concentration of Gd³⁺ was 1 mM. After 30 min incubation, the longitudinal relaxation rate constant (T1) was measured using an inversion recovery pulse sequence (180°-dₜ-90°-acquire) at 298 K. Arrayed data [n(dt)=10] were processed using Acorn NUTS software and fit to a three-parameter model. The inverse of the T1 data were used to determine the longitudinal relaxation rate constants (R1) imparted by the polyplexes. Solution concentration data for beacons Gd3a and Gd3b, as well as the Magnevist control were used to calculate relaxivity (r1) by generating a curve fit to (1/T1)=[Gd³⁺](r₁)+b, where b=(1/T₁) for the working solvent, water.

The Eu³⁺ polymer analogs can be visualized on the nm/μm scale within cells, while the Gd³⁺ analogs provide the ability to monitor nucleic acid delivery within tissues on the sub-mm scale. Their relaxivity (r1) and relaxation rate constants (R1) were determined, which data of the polymers (r1, determined by plotting 1/T1 versus concentration) or polyplexes (R1, equivalent to 1/T1) can be used to understand the contrast enhancement of the delivery beacons.

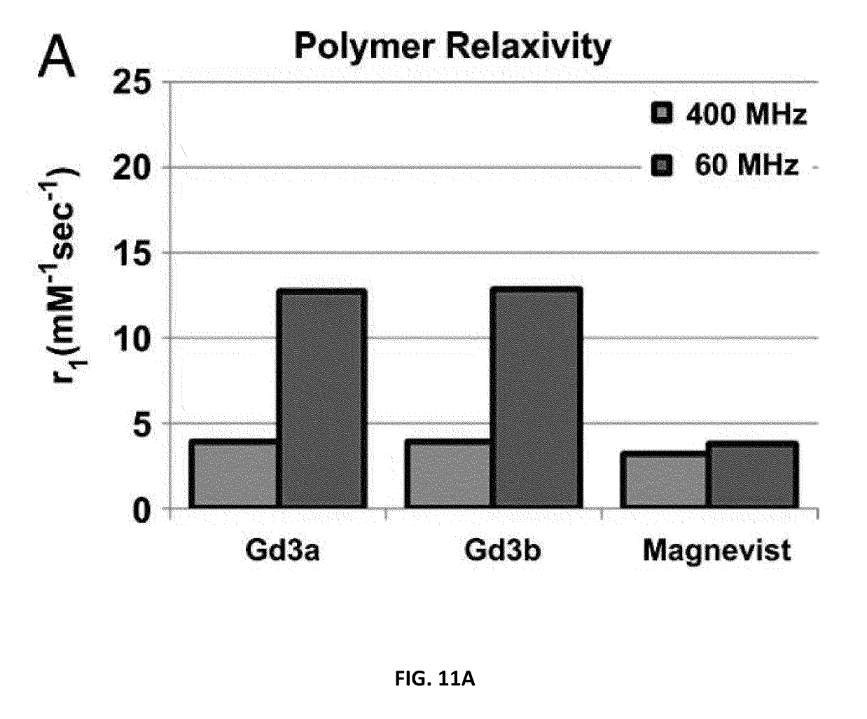

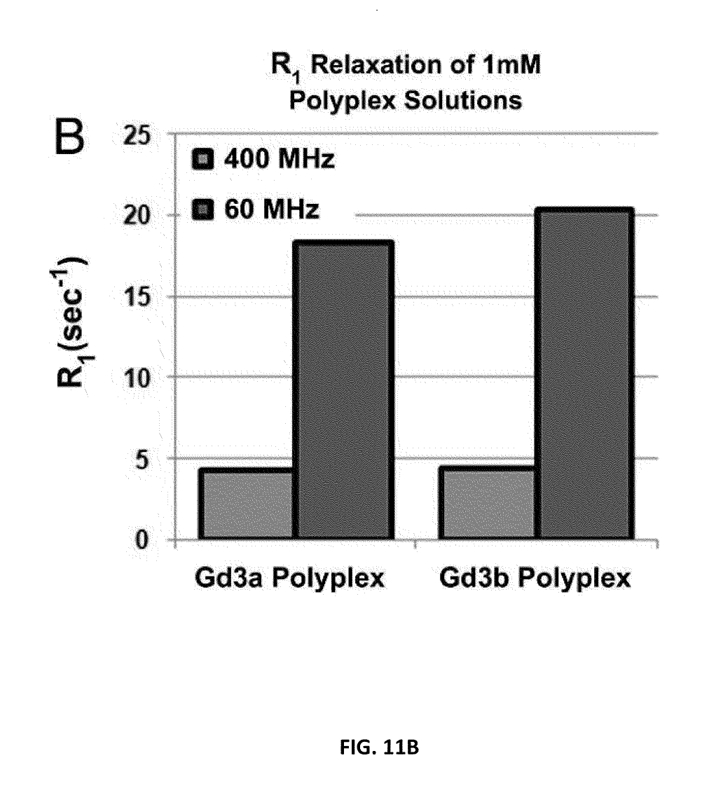

As shown in FIGS. 11A and 11B, the relaxivities and relaxation rate constants of water solutions containing dissolved polymer and polyplexes, respectively, are provided. In particular, FIG. 11A shows the relaxivity (r1) of aqueous solutions containing free polymer (Gd3a and Gd3b only) and Magnevist at 400 MHz (9.4 T) and 60 MHz (1.41 T). Relaxivity was calculated from four concentrations (5, 2.5, 1.25, and 0 mM) of each agent in water. FIG. 11B shows the relaxation rate constants (R1) of polyplexes formed with Gd3a and Gd3b at a concentration of 1 mM at 400 MHz (9.4 T) and 60 MHz (1.41 T).

The relaxivity experiments were performed on both the free Gd3a and Gd3b polymers (FIG. 11A) in solution and commercial contrast agent, Gd³⁺-DTPA (Magnevist) at two different magnetic field strengths (60 MHz/1.41 T and 400 MHz/9.4 T at 37° C.). Also, the ability of Gd3a and Gd3b polyplexes to enhance longitudinal water proton relaxation was tested (FIG. 11B). The relaxivities of both polymers in solution were higher than the clinically-used chelate Gd³⁺DTPA, and a dramatic enhancement was observed at 1.41 T, likely due to the slower rotational correlation time of the delivery beacons. When comparing the longitudinal relaxation rates (R1) of free polymer to polyplexes at 1.41 T (Gd³⁺ concentration is identical) a dramatic relaxivity increase was observed. This difference in relaxivity between free polymer and polyplex could provide a unique method to track nucleic acid release within tissues.

### Example VIII

MRI of Transfected HeLa Cells. To further investigate the MRI contrast capabilities of the Gd³⁺-chelate materials, HeLa cells were transfected with the polyplexes formulated with Gd3a and Gd3b. Specifically, twenty-four hours before transfection, HeLa cells were seeded in 10 flasks (with a surface area of 75 cm2) at a density of 4.0×10⁶ cells per flask. Polyplexes were prepared 1 h before transfection using βCMV pDNA and the respective polymer at an N/P ratio of 40. Cells were transfected in 15 mL Opti-MEM using 80 μg pDNA/flask. Nontransfected cells were subjected to the same media changes as the transfected cells. Four hours after transfection, 15 mL of DMEM containing 10% FBS was added to each well. Twenty-four hours after transfection, cells were aspirated of media, washed extensively with PBS and collected by trypsination. Cells were pelleted and resuspended in PBS twice, then pelleted again in 0.5 mL Eppendorf tubes for analysis.

All MR images were acquired with a Bruker Avance III NMR spectrometer equipped with a Micro2.5 imaging probe featuring a 3 cm rf coil. The images were acquired using a RARE-inversion recovery T1-weighted pulse sequence. Acquisition parameters are as follows: TR (repetition time)=3,500 msec; TI (inversion time)=1,200 msec; TE (echo time)=8.5 msec; FOV=3×3 cm; and resolution=256×256. T1 measurements of these same cell pellets and controls were carried out using the same parameters on a Bruker AMX-400 MHz spectrometer. For the T1 experiments, the HeLa cell pellets used for the MRI experiment were suspended in PBS buffer to make turbid solutions that were deposited into NMR tubes and allowed to rest for 24 h. On settling, each tube contained approximately 4 cm of sedimented HeLa cell pellets. NMR tubes were placed so the entirety of the HeLa pellet was within the NMR coil. The longitudinal relaxation rate constant (T1) for each pellet and the control solutions were measured using an inversion recovery pulse sequence (180°-dₜ-90°-acquire) at 298 K. Arrayed data [n(dₜ)=10] was processed using Acorn NUTS software and fit to a three-parameter model.

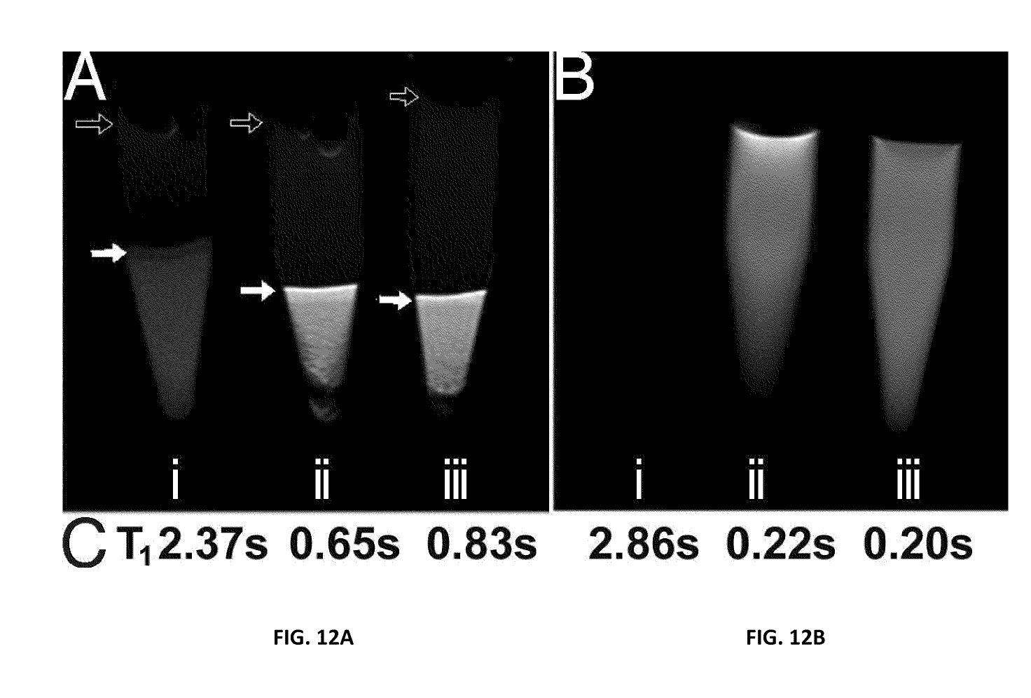

FIGS. 12A-B show the MRI data for cells transfected with Gd3a/pDNA and Gd3b/pDNA polyplexes. The cells were thoroughly washed and gravity pelleted into Eppendorf tubes. FIG. 12A shows transfected cell pellets and controls: (Ai) Pellet of untreated HeLa cells, (Aii) cell pellets transfected with Gd3a polyplexes (N/P=40), and (Aiii) cells transfected with Gd3b polyplexes (N/P=40). Solid arrows indicate the buffer-cell interface in each sample. The perturbations at the top of the buffer (open arrows) are due to bubbles at the buffer-air interface. The darker spots in the cell pellet are due to cell density gradients. FIG. 12B shows MR images of (Bi) PBS buffer, (Bii) Gd3a ([Gd³⁺]=1 mM), and (Biii) Gd3b ([Gd³⁺]=1 mM) using the same pulse parameters as above. PBS is completely dark because of the high T1 contrast relative to Gd3a/b solutions. T1 quantification of cell and solution samples imaged are provided below the figures. Cell samples were allowed to settle into NMR tubes for 24 h and then analyzed with an inversion recovery pulse sequence.

More specifically as shown in FIG. 12A, tubes containing live transfected or untransfected cells were subjected to a T1-weighted pulse sequence, imaged for tissue contrast, and then analyzed for water 1H T1 values. Untransfected cells that do not contain Gd3a or Gd3b demonstrate minimal image brightness/contrast FIG. 12A (i), and this control cell pellet has a higher T1 value (2.4 s) than cells transfected with Gd3a and Gd3b polyplexes. Cells transfected with Gd3a and Gd3b (FIG. 12A (ii) and (iii) show a clear enhancement in the cellular image brightness and substantially lower T1.

FIG. 12B shows the T1 weighted images, using the same pulse conditions as in FIG. 12A, with 1 mM (based on the Gd³⁺ concentration) solutions FIG. 12B (ii) and (iii) versus a PBS control. Because the beacon solutions are so bright relative to the saline control, the control becomes unobservable. This is confirmed by their T1 analysis at 9.4 T, which agrees with the MR imaging experiment.

These promising data clearly show the potential of the imaging beacons to trace the spatial and temporal delivery of nucleic acids within bulk cells and/or tissues. Thus, the nucleic acid delivery beacons show great promise in the development of innovative techniques to understand the delivery, trafficking mechanisms, and fate of non-viral delivery vehicles on differing biological scales. The lanthanide metals offer many unique properties, which can be exploited to enhance these imaging techniques. The Eu³⁺-chelated polymers can be visualized by their luminescence within cultured cells on the nm/μm scale for tracking the intracellular delivery of polyplexes. For larger-scale MR imaging, the paramagnetic nature of the Gd³⁺-chelated polymers offers a safe and noninvasive probe for following nucleic acid delivery within tissues on the sub-mm scale. These scaffolds have potential for monitoring in vivo delivery in a spatial and temporal manner and can be used intact (without removal of the imaging probe) due to their nontoxic and effective delivery profile. Indeed, these creative and powerful materials can be broadly applied and exploited by researchers for the discovery of unique nucleic acid drug/vehicle conjugates and to understand and treat many devastating diseases.

The present invention has been described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. The description of the invention provided is merely exemplary in nature and, thus, variations that do not depart from the essence of the invention are intended to be within the scope of the invention.

1. An unchelated polymer with a positive charge capable of binding with one or more functional group or metal and having an original repeating unit comprising: 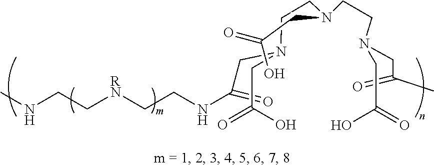
wherein R is chosen from a hydrogen atom, or a methyl or t-butoxycarbonyl (Boc) group;
and n ranges from 2 to 10,000,000.

2. A polymeric imaging beacon comprising repeating units of metal chelates within an oligoamine backbone, the repeating units comprising: 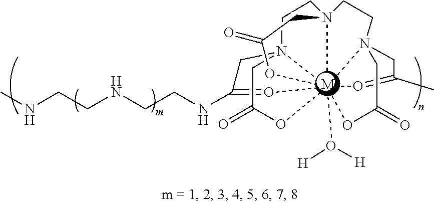
wherein n ranges from 2 to 10,000,000;
M is a metal capable of exhibiting an imaging functionality for an imaging modality; and
the oligoamine backbone comprises from 1 to 8 ethlyeneamines and is chosen from diethyleneamine, triethylenediamine, tetraethylenetriamine, pentaethylenetetraamine, hexaethylenepentamine, heptaethylenehexamine, octaethyleneheptamine, or nonethyleneoctaamine.

3. The polymeric imaging beacon of claim 2, wherein M is a metal chosen from copper, manganese, iron, or a lanthanide metal chosen from lanthanum, cerium, praseodymium, neodymium, promethium, samarium, europium, gadolinium, terbium, dysprosium, holmium, erbium, thulium, ytterbium, and lutetium.

4. The polymeric imaging beacon of claim 2 having a structure defined by: 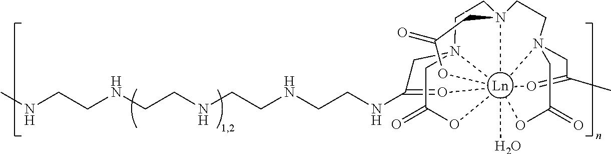
wherein n ranges from 2 to 10,000,000;
wherein Ln is a lanthanide metal chosen from lanthanum, cerium, praseodymium, neodymium, promethium, samarium, europium, gadolinium, terbium, dysprosium, holmium, erbium, thulium, ytterbium, and lutetium; and the oligoamine backbone comprises a tetraethylenetriamine or pentaethylenetetraamine to form an imaging beacon chosen from:
poly[(tetraethylenetriamine)amido(Ln³⁺)diethylenetriaminetriacetate] or poly[(pentaethylenetetramine)amido(Ln³⁺)-diethylenetriaminetriacetate].

5. The polymeric imaging beacon of claim 2, wherein M is a lanthanide metal chosen from gadolinium, europium, or terbium.

6. A polyplex comprising:
a) a polymeric portion comprising repeating units of metal chelates within an oligoamine backbone, the repeating units comprising: 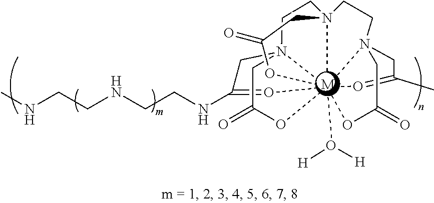
wherein n ranges from 2 to 10,000,000;
M is a metal capable of exhibiting an imaging functionality for an imaging modality; and
the oligoamine backbone comprises from 1 to 8 ethlyeneamines and is chosen from diethyleneamine, triethylenediamine, tetraethylenetriamine, pentaethylenetetraamine, hexaethylenepentamine, heptaethylenehexamine, octaethyleneheptamine or nonethyleneoctaamine; and
b) a molecule in complex with the polymeric portion chosen from a polynucleotide, a small molecule drug, a biologic, a nucleic acid, a protein, and a peptide sequence.

7. The polyplex of claim 6, wherein the repeating units comprise: 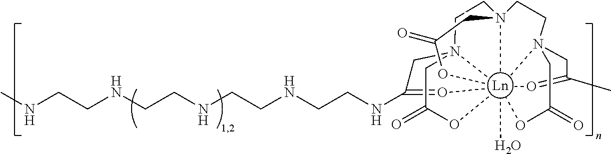
wherein n ranges from 2 to 10,000,000;
wherein Ln is a lanthanide metal chosen from lanthanum, cerium, praseodymium, neodymium, promethium, samarium, europium, gadolinium, terbium, dysprosium, holmium, erbium, thulium, ytterbium, and lutetium; and the oligoamine backbone comprises a tetraethylenetriamine or pentaethylenetetraamine to form an imaging beacon chosen from:
poly[(tetraethylenetriamine)amido(Ln³⁺)diethylenetriaminetriacetate] or poly[(pentaethylenetetramine)amido(Ln³⁺)-diethylenetriaminetriacetate].

8. The polyplex of claim 6, wherein M is a lanthanide metal chosen from gadolinium, europium, or terbium.

9. The polyplex of claim 6, wherein the molecule in complex with the polymeric portion is a nucleic acid chosen from pDNA, siRNA, or an oligodeoxynucleotide.

10. A method of delivering a polynucleotide into a cell comprising:
administering in vivo or contacting with a cell in vitro a polyplex comprising:
a) a polymeric portion comprising repeating units of metal chelates within an oligoamine backbone, the repeating units comprising: 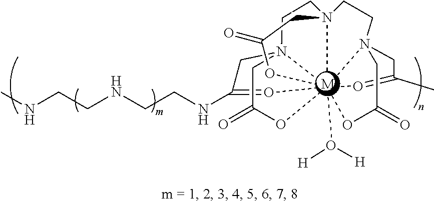
wherein n ranges from 2 to 10,000,000;
M is a metal capable of exhibiting an imaging functionality for an imaging modality; and
the oligoamine backbone comprises from 1 to 8 ethlyeneamines and is chosen from diethyleneamine, triethylenediamine, tetraethylenetriamine, pentaethylenetetraamine, hexaethylenepentamine, heptaethylenehexamine, octaethyleneheptamine or nonethyleneoctaamine; and
b) a molecule in complex with the polymeric portion chosen from a polynucleotide, a small molecule drug, a biologic, a protein, and a peptide sequence.

11. The method of claim 10, wherein the administering is of a polyplex, wherein the repeating units comprise: 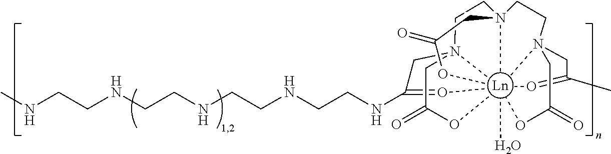
wherein n ranges from 2 to 10,000,000;
wherein Ln is a lanthanide metal chosen from lanthanum, cerium, praseodymium, neodymium, promethium, samarium, europium, gadolinium, terbium, dysprosium, holmium, erbium, thulium, ytterbium, and lutetium; and the oligoamine backbone comprises a tetraethylenetriamine or pentaethylenetetraamine to form an imaging beacon chosen from:
poly[(tetraethylenetriamine)amido(Ln³⁺)diethylenetriaminetriacetate] or poly[(pentaethylenetetramine)amido(Ln³⁺)-diethylenetriaminetriacetate].

12. The method of claim 11, wherein the administering involves targeting a selected number of cells, and wherein the polynucleotide is chosen from pDNA, siRNA, or an oligodeoxynucleotide and wherein uptake by a desired portion of the cells is accomplished.

13. The method of claim 11, further comprising visualizing by luminescence the polyplex or part thereof by microscopy for tracking intracellular delivery of the polyplex.

14. The method of claim 14, wherein Eu3+, Tb3+, or Sm3+ is the lanthanide which enables luminescence of the polyplex and visualization by microscopy.

15. The method of claim 11, further comprising imaging the polyplex or part thereof by magnetic resonance imaging (MRI) for following polynucleotide delivery within cultured cells or tissues, or in vivo.

16. The method of claim 15, wherein Gd3+ is the lanthanide which enables imaging of the polyplex by MRI.

17. A kit comprising one or more polymeric imaging beacon comprising repeating units of metal chelates within an oligoamine backbone, the repeating units comprising: 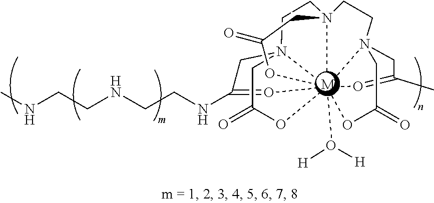
wherein n ranges from 2 to 10,000,000;
M is a metal capable of exhibiting an imaging functionality for an imaging modality; and
the oligoamine backbone comprises from 1 to 8 ethlyeneamines and is chosen from diethyleneamine, triethylenediamine, tetraethylenetriamine, pentaethylenetetraamine, hexaethylenepentamine, heptaethylenehexamine, octaethyleneheptamine or nonethyleneoctaamine; and
one or more biologically active molecules capable of forming a complex with the beacon.
